(19) United States
(12) Patent Application Publication
(10) Pub. No.: US 2024/0407945 A1
(43) Pub. Date: Dec. 12, 2024

(54) OPHTHALMIC DEVICE

(71) Applicant: aVISI Ltd., Tel-Aviv (IL)

(72) Inventor: Nir BETSER, Tel-Aviv (IL)

(73) Assignee: aVISI Ltd., Tel-Aviv (IL)

(21) Appl. No.: 18/702,359

(22) PCT Filed: Oct. 27, 2022
(86) PCT No.: PCT/IL2022/051137

§ 371 Date: Apr. 18, 2024

Related U.S. Application Data
(60) Provisional application No. 63/272,315, filed on Oct. 27, 2021.

(51) Int. Cl. A61F 9/00

(52) U.S. Cl. CPC A61F 9/0017; A61F 2220/0008; A61F 2230/0006; A61F 2230/0008; A61F 2250/0037

(57) ABSTRACT

An ophthalmic device including: a body having: a posterior surface which is adjacent to an eye surface when the ophthalmic device is worn; an anterior surface defining a height of the body above the eye surface; a retention portion, configured to reside between an eyelid and the eye surface, the retention portion bordered by a portion of the anterior surface, and having a height difference of at least 1 mm over at most a 1 mm distance.

### RELATED APPLICATIONS

This application claims the benefit of priority of U.S. Provisional Patent Application No. 63/272,315 filed on 27 Oct. 2021, the contents of which are incorporated herein by reference in their entirety.

### FIELD AND BACKGROUND OF THE INVENTION

The present invention, in some embodiments thereof, relates to an ophthalmic device and, more particularly, but not exclusively, to a drug eluting ophthalmic device.

U.S. Patent No. U.S. Pat. No. 9,724,230 discloses “Dry eye treatment apparatus and methods are described herein which generally includes a patch or strip affixed to the skin of the upper and/or lower eyelids to deliver heat or other forms of energy, pressure, drugs, moisture, etc. (alone or in combination) to the one or more meibomian glands contained within the underlying skin. The treatment strip or strips include one or more strips configured to adhere to an underlying region of skin in proximity to one or both eyes of a subject such that the one or more strips allow for the subject to blink naturally without restriction from the one or more strips. Moreover, the one or more strips may be configured to emit energy to the underlying region of skin and where the one or more strips are shaped to follow a location of one or more meibomian glands contained within the underlying region of skin.”

U.S. Patent No. U.S. Pat. No. 5,137,728 “An ocular insert for use behind an eyelid, comprising a substantially circular disc having a concave posterior surface and a convex anterior surface wherein the radius of curvature of said posterior surface is less than the radius of curvature of the sclera of the eye, the center thickness of said insert is less than about 1.5 mm and the ratio of the radius of curvature of said posterior surface to the radius of curvature of the sclera is less than about 0.8 mm wherein incorporated in said ocular insert a medicinal agent is incorporated therein.”

Additional background art includes U.S. Pat. Nos. 6,217,896, 8,679,078, 3,416,530, 3,828,777, 4,014,335, 5,773,021, and 10,010,502.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. An ophthalmic device comprising:
a body having:
a posterior surface which is adjacent to an eye surface when said ophthalmic device is worn;
an anterior surface defining a height of said body above said eye surface;
a retention portion, configured to reside between an eyelid and said eye surface, said retention portion bordered by a portion of said anterior surface, and having a height difference of at least 1 mm over at most a 1 mm distance.

Example 2. The ophthalmic device according to example 1, wherein said body is not hollow.

Example 3. The ophthalmic device according to example 1 or example 2, wherein said retention portion is made of a continuous material.

Example 4. The ophthalmic device according to any one of examples 1-3, wherein said body has an oval or round form.

Example 5. The ophthalmic device according to any one of examples 1-4, wherein said body is symmetrical in its longitudinal axis and in its transversal axis.

Example 6. The ophthalmic device according to any one of examples 1-5, wherein said retention portion has a height of at least 1 mm for an area of at least 1 mm2.

Example 7. The ophthalmic device according to any one of examples 1-6, wherein said height difference is in a direction moving away from an edge of said body.

Example 8. The ophthalmic device according to any one of examples 1-7, wherein said distance is measured along a surface of said portion of said anterior surface.

Example 9. The ophthalmic device according to any one of examples 1-8, wherein said height difference is measured between said retention portion an edge of said body.

Example 10. The ophthalmic device according to any one of examples 1-9, wherein said height difference is measured between a first portion of said retention portion and a second portion of said retention portion.

Example 11. The ophthalmic device according to any one of examples 1-10, wherein said retention portion allows said device to entirely reside between an eyelid and an eye surface.

Example 12. The ophthalmic device according to example 7 or example 9, wherein said edge of said body is a leading edge of said body disposed adjacent to an opening of an eyelid when said ophthalmic device is worn underneath said eyelid.

Example 13. The ophthalmic device according to any one of examples 1-12, wherein said body has a maximal height above said eye surface of 3 mm.

Example 14. The ophthalmic device according to any one of examples 1-13, wherein said retention portion has a slope of at least 45° for at least a length of 1 mm along said anterior surface.

Example 15. The ophthalmic device according to any one of examples 1-14, wherein said body comprises a first portion and a second portion, where said first portion degrades more rapidly than said second portion when said body is in residence on said eye surface.

Example 16. The ophthalmic device according to example 15, wherein said second portion forms part or all of said retention portion.

Example 17. The ophthalmic device according to any one of examples 1-16, wherein said body has an average thickness of at least 400 microns.

Example 18. The ophthalmic device according to any one of examples 1-17, wherein said body has a maximal thickness of at least 400 microns.

Example 19. The ophthalmic device according to any one of examples 1-18, wherein said height difference is of at least 1.5 mm.

Example 20. The ophthalmic device according to any one of examples 1-19, wherein, when in residence on said eye surface, said anterior surface is adjacent to an inner surface of said eyelid, and said retention portion is held in position by a tarsal plate.

Example 21. The ophthalmic device according to any one of examples 1-20, wherein said body comprises therapeutic material and is configured to elute the therapeutic material into said eye when residing on said eye surface.

Example 22. The ophthalmic device according to any one of examples 1-21, wherein an extent of said body is 2-8 mm.

Example 23. The ophthalmic device according to any one of examples 1-22, wherein said body is elongate.

Example 24. The ophthalmic device according to any one of examples 1-23, wherein said body is rotationally symmetrical around an axis connecting said anterior surface and said posterior surface.

Example 25. The ophthalmic device according to any one of examples 1-24, wherein said retention portion includes a maximal height portion of said body.

Example 26. The ophthalmic device according to any one of examples 1-25, wherein height of said body reduces moving from said retention portion towards edges of said device.

Example 27. The ophthalmic device according to any one of examples 1-26, wherein said posterior surface is concave.

Example 28. The ophthalmic device according to any one of examples 1-27, wherein a softness of said device is 0.3-1.5 Mpa.

Example 29. The ophthalmic device according to any one of examples 1-28, wherein said anterior surface is convex.

Example 30. The ophthalmic device according to any one of examples 1-29, wherein said device is configured to move with respect to said eye surface, during wear.

Example 31. The ophthalmic device according to example 30, wherein said posterior surface has sufficiently low suction forces onto said eye surface to enable said movement.

Example 32. The ophthalmic device according to any one of examples 1-31, wherein said posterior surface has low curvature.

Example 33. The ophthalmic device according to any one of examples 1-32, wherein said posterior surface is convex.

Example 34. The ophthalmic device according to any one of examples 1-33, wherein a radius of curvature of said anterior surface is smaller than a radius of curvature of said posterior surface.

Example 35. The ophthalmic device according to any one of examples 1-34, wherein said device is configured to reside on said eye surface for at least 15 minutes.

Example 36. The ophthalmic device according to any one of examples 1-35, wherein an edge region of said body, at less than 0.5 mm from an edge of said body, has a thickness of less than 50 microns.

Example 37. The ophthalmic device according to any one of examples 1-36, wherein said posterior surface comprises mucoadhesive material.

Example 38. The ophthalmic device according to any one of examples 1-37, wherein said anterior surface is smooth and/or comprises lubricious material.

Example 39. The ophthalmic device according to any one of examples 1-38, wherein said body comprises material which disintegrates within an eye.

Example 40. The ophthalmic device according to any one of examples 1-39, wherein said body comprises one or both of biodegradable and bioerodable material.

Example 41. The ophthalmic device according to any one of examples 1-40, wherein said posterior surface comprises one or more cavity.

Example 42. The ophthalmic device according to any one of examples 1-41, wherein said posterior surface comprises one or more protrusion.

Example 43. The ophthalmic device according to any one of examples 1-42, wherein said device comprises one or more electronic element.

Example 44. The ophthalmic device according to any one of examples 1-43, wherein said device is supplied in dry or semi-hydrated form.

Example 45. The ophthalmic device according to any one of examples 1-44, wherein said body comprises more than one layer with different material characteristics.

Example 46. The ophthalmic device according to any one of examples 1-45, wherein said body comprises one or more of:
Hydroxy Propyl Cellulose (HPC); Hydroxy Propyl Methyl Cellulose (HPMC); Carboxymethyl cellulose (CMC); PolyVinyl Alcohol (PVOH); PolyEthylene Glycol (PEG); Cellulose Acetate (CA); Polyvinyl alcohol-polyethylene glycol graft-copolymer (Kollicoat); Poly Acrylic Acid (Carbopol or Carbomer for example); Hyaluronic Acid; Ethyl cellulose; Propylene Glycol; Tri Ethyl Citrate (TEC); Glycerol; Dextran; and combinations thereof.

Example 47. The ophthalmic device according to any one of examples 1-46, where said device comprises one or more anchor configured to hold said device on said eye surface.

Example 48. The ophthalmic device according to example 47, wherein said anchor comprises sharp edges of said body.

Example 49. The ophthalmic device according to any one of examples 1-48, wherein a radius of curvature of one or more edge of said body is less than 100 microns.

Example 50. The ophthalmic device according to any one of examples 1-49, wherein said retention portion has an anterior surface having a slope of at least 45° for at least a length of 1 mm.

Example 51. The ophthalmic device according to any one of examples 1-50, wherein said device comprise a section comprising a length of at least 1 mm where a height between said anterior surface and said posterior surface is constant.

Example 52. The ophthalmic device according to any one of examples 1-51, wherein said retention portion allows said device to remain between an eyelid and an eye surface.

Example 53. The ophthalmic device according to any one of examples 1-52, wherein said retention portion prevents said device from sliding under an upper eyelid.

Example 54. An ophthalmic device, comprising:

A body comprising:
a. a posterior surface which is adjacent to an eye surface when said ophthalmic device is worn;
b. an anterior surface which is adjacent to an eyelid when said ophthalmic device is worn;
c. a retention portion defined by heights between said posterior surface and said anterior surface;
wherein a first height of said heights measured from a first point on said posterior surface to a corresponding point on said anterior surface differs by at least 1 mm, from a second height of said heights measured from a second point on said posterior surface to a corresponding point on said anterior surface, said second point being distanced at most 1 mm from said first point.

Example 55. An ophthalmic device, comprising:

A body comprising:
a. a posterior surface which is adjacent to an eye surface when said ophthalmic device is worn;
b. an anterior surface which is adjacent to an eyelid when said ophthalmic device is worn;
c. a retention portion defined by heights between said posterior surface and said anterior surface;
wherein at least some of said heights between said posterior surface and said anterior surface differ from each other by at least 1 mm when measured from two points on said posterior surface distanced from each other by at most 1 mm.

Example 56. An ophthalmic device comprising:
a body having:
   a posterior surface;
an anterior surface defining a height of said body above a planar surface on which said ophthalmic device is disposed in a relaxed configuration, said posterior surface adjacent to said planar surface; and
a retention portion, configured to reside between an eyelid and an eye surface, said retention portion bordered by a portion of said anterior surface, and having a height difference of at least 1 mm over at most a 1 mm distance.

Example 57. An ophthalmic device comprising:
a body comprising:
a posterior surface which is adjacent to an eye surface when said ophthalmic device is worn;
an anterior surface defining a height of said body above said eye surface;
a retention portion, configured to reside between an eyelid and said eye surface, said retention portion bordered by a portion of said anterior surface, said portion of said anterior surface having a slope of at least 45° with respect to said eye surface for at least a length of 1 mm along said anterior surface.

Example 58. An ophthalmic device comprising:
a body comprising:
a posterior surface which is adjacent to an eye surface when said ophthalmic device is worn;
an anterior surface defining a height of said body above a planar surface on which said ophthalmic device is disposed in a relaxed configuration, said posterior surface adjacent to said planar surface; and
a retention portion, configured to reside between an eyelid and said eye surface, said retention portion bordered by a portion of said anterior surface, said portion of said anterior surface and having a slope of at least 45° with respect to said planar surface for at least a length of 1 mm along said anterior surface.

Example 59. An ophthalmic device comprising:
a body having a first portion and a second portion, where said first portion degrades more rapidly than said second portion when said body is in residence on an eye surface.

Example 60. A method of treating an eye, comprising:
a. inserting an ophthalmic device according to example 1, 54, 55, 56, 57, 58 or 59, between an eyelid and a surface of said eye;
b. releasing at least one medication into said eye.

Example 61. The method according to example 60, wherein said inserting comprises inserting under an inferior eyelid.

Example 62. The method according to example 60 or example 61, wherein said inserting comprises inserting under a superior eyelid.

Example 63. The method according to any one of examples 60-62, wherein said inserting comprises inserting behind a tarsal plate in said eye.

Example 64. The method according to any one of examples 60-63, wherein said inserting comprises positioning said device on a bulbar conjunctiva and at least partially underneath said eyelid.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such inspecting objects, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings, in some embodiments, like numerals refer to like elements. For example, the element 616 in FIG. 6A, in some embodiments, corresponding to the element 1316 in FIG. 13A.

In the drawings:

FIG. 1 is a method of use of an ophthalmic device, according to some embodiments of the invention;

FIG. 2 is a method of treatment using an ophthalmic device, according to some embodiment of the invention;

FIG. 3A is a simplified schematic cross sectional view of an eye;

FIG. 3B is a simplified schematic cross sectional view of an ophthalmic device in position on an eye surface, when the eye is closed, according to some embodiments of the invention;

FIG. 3C is a simplified schematic cross sectional view of an ophthalmic device in position on an eye surface, when the eye is open, according to some embodiments of the invention;

FIG. 3D is a simplified schematic top view of an ophthalmic device in position on an eye surface, according to some embodiments of the invention;

FIG. 4A is a simplified schematic top view of an ophthalmic device in position on an eye surface, according to some embodiments of the invention;

FIG. 4B is a simplified schematic cross sectional view of an ophthalmic device in position on an eye surface, when the eye is closed, according to some embodiments of the invention;

FIG. 5 is a method of use of an ophthalmic device, according to some embodiments of the invention;

FIG. 6A is a simplified schematic top view of an ophthalmic device 600 in position on an eye surface underneath an eyelid, according to some embodiments of the invention;

FIG. 6B is a simplified schematic cross section of an ophthalmic device, according to some embodiments of the invention;

FIG. 7 is a simplified schematic illustrating cross sections of portions of devices, according to some embodiments of the invention;

FIGS. 8, 9, 10, 11 and 12 are simplified schematic top views of devices, according to some embodiments of the invention;

FIG. 13A is a simplified schematic view of an ophthalmic device, according to some embodiments of the invention;

FIG. 13B is a simplified schematic cross sectional view of an ophthalmic device, according to some embodiments of the invention;

FIG. 13C is a simplified schematic top view of an ophthalmic device, according to some embodiments of the invention;

FIG. 14 is a simplified schematic cross sectional view of an ophthalmic device, according to some embodiments of the invention;

FIG. 15A is a simplified schematic top view of an ophthalmic device, according to some embodiments of the invention;

FIG. 15B is a simplified schematic view of an ophthalmic device, according to some embodiments of the invention;

FIG. 15C is a simplified schematic cross sectional view of an ophthalmic device, according to some embodiments of the invention;

FIG. 15D is a simplified schematic cross sectional view of an ophthalmic device, according to some embodiments of the invention;

FIGS. 16, 17, 18, 19 and 20 are simplified schematic top views of devices, according to some embodiments of the invention;

FIGS. 21, 22, 23 and 24 are simplified schematic cross sections of devices, according to some embodiments of the invention;

FIG. 25A is a simplified schematic cross sectional view of a device positioned on an eye surface, according to some embodiments of the invention;

FIG. 25B is a simplified schematic cross sectional view of a device, according to some embodiments of the invention;

FIG. 26 is a simplified schematic cross sectional view of a device, according to some embodiments of the invention;

FIG. 27A is a simplified schematic top view of an ophthalmic device, according to some embodiments of the invention;

FIG. 27B is a simplified schematic cross sectional view of an ophthalmic device, according to some embodiments of the invention;

FIG. 27C is a simplified schematic cross sectional view of a device 2700, according to some embodiments of the invention;

FIG. 28 is a process of ophthalmic device degradation, according to some embodiments of the invention;

FIG. 29 is a simplified schematic cross sectional view of a device, according to some embodiments of the invention;

FIGS. 30A-B are simplified schematic cross sectional views of a device, according to some embodiments of the invention;

FIG. 31A is a simplified schematic cross section view of an ophthalmic device on an eye surface, according to some embodiments of the invention;

FIG. 31B is a simplified schematic view of an ophthalmic device, according to some embodiments of the invention;

FIG. 31C is a simplified schematic to view of an ophthalmic device, according to some embodiments of the invention;

FIGS. 32, 33, 34, 35, 36 and 37 are simplified schematic cross section views of portions of ophthalmic devices, according to some embodiments of the invention;

FIGS. 38, 39, 40, 41, 42, 43 and 44 are simplified schematic cross sectional views of devices, according to some embodiments of the invention;

FIGS. 45, 46, 47, 48, 49 and 50 are simplified schematic cross sectional views of double layer devices according to some embodiments of the invention;

FIGS. 51, 52, 53, 54, 55, 56, 57 and 58 are simplified schematic cross sectional views of multi-layer devices, according to some embodiments of the invention;

FIG. 59A is a simplified schematic cross section of an ophthalmic device, according to some embodiments of the invention;

FIG. 59B is a simplified schematic cross section of an ophthalmic device, according to some embodiments of the invention;

FIG. 60 is a simplified schematic cross section of an ophthalmic device, according to some embodiments of the invention;

FIG. 61 is a simplified schematic cross section of an ophthalmic device, according to some embodiments of the invention;

FIG. 62A is a simplified schematic cross sectional view of a portion of an eye surface, according to some embodiments of the invention;

FIG. 62B is a simplified schematic cross section view of an ophthalmic device on an eye surface, according to some embodiments of the invention;

FIG. 62C is a simplified schematic cross section view of an ophthalmic device on an eye surface, according to some embodiments of the invention;

FIGS. 63A-C are simplified schematic cross sectional views of a portion of a device on an eye surface, according to some embodiments of the invention;

FIGS. 64, 65 and 66 are simplified schematic cross sectional views of multi-layer devices, according to some embodiments of the invention;

FIGS. 67A-F are simplified schematic cross sections of a portion of a device as residence time of the device progresses, according to some embodiments of the invention;

FIG. 68 is a flowchart of a disintegration progression of a device, according to some embodiments of the invention;

FIGS. 69A-C are simplified schematic cross sections of a device as residence time of the device progresses, according to some embodiments of the invention;

FIGS. 70A-D are simplified schematic cross sections of a device as residence time of the device progresses, according to some embodiments of the invention;

FIG. 71 is a simplified schematic cross section of a device, according to some embodiments of the invention;

FIG. 72 is a simplified schematic cross section of a device, according to some embodiments of the invention;

FIGS. 73A-B are simplified schematic cross sectional views of a device on an eye surface 7304, according to some embodiments of the invention;

FIGS. 74A-D are simplified schematic cross sectional views of a film, according to some embodiments of the invention

FIG. 75 is a method of manufacture, according to some embodiments of the invention; and

FIGS. 76A-C are simplified schematic cross sectional views of a device, according to some embodiments of the invention.

### DESCRIPTION OF SPECIFIC EMBODIMENTS OF THE INVENTION

The present invention, in some embodiments thereof, relates to an ophthalmic device and, more particularly, but not exclusively, to a drug eluting ophthalmic device.

### Overview

A broad aspect of some embodiments relates to an ophthalmic device for treatment and/or protection of the eye which, when positioned on an eye remains within the eye. For example, without moving from a region of the eye to which it has been positioned and/or without being expelled from the eye during a treatment time period. Where, in some embodiments, the device is positioned underneath the eyelid and/or on a portion of the eyeball and/or on a portion of the eyeball that does not reach the iris. Whilst, in some embodiments, the device remains comfortable for a user to wear.

In some embodiments, the device is in residence on the eye and/or remains in position (e.g. underneath an eyelid) during regular activities of a subject, for example, one or more of, blinking, tearing, crying, sweating, washing (e.g. showering), walking, exercising, sleeping. In some embodiments, the device is in residence on the eye and/or remains in position while the eyes are mainly closed e.g. during sleeping and/or for a semi-conscious or unconscious patient.

In some embodiments, the ophthalmic device includes one or more therapeutic agent and/or other compound/s which are released (e.g. elute from the device) while the device resides in the eye.

An aspect of some embodiments relates to an ophthalmic device which is configured to remain in position, underneath an eyelid of an eye. In some embodiments, optionally, part of the ophthalmic device remains in position underneath an eyelid of an eye while another part of the ophthalmic device extends outside the eyelid, optionally without reaching the iris. In some embodiments, the ophthalmic device is configured to completely reside and remain between the surface of the eye and the eyelid. In some embodiments, the ophthalmic device does not comprise any part that extends outside the eyelid. In some embodiments, the device is configured to reside on a bulbar conjunctiva and underneath the eyelid. For example, during opening and closing of the eye (e.g. blinking). In some embodiments, the ophthalmic device includes a retention portion which is sized and/or shaped to maintain the device underneath the eyelid. In some embodiments, the retention portion is the whole ophthalmic device. Where, in some embodiments, the retention portion is sufficiently thick and/or presents a sufficient height and/or a sufficiently high slope to eye tissue that the device remains in position underneath the eyelid. For example, an anterior surface of the device presents a sufficient height and/or slope to eye tissue e.g. to an edge of the eyelid and/or to the tarsal plate. In some embodiments, the thickness and/or height extends sufficiently in one or more direction. In some embodiments, the retention portion includes an anterior surface which presents a sufficient slope to eye tissue.

In some embodiments, properties of the retention portion are dependent on one or more of a shape of the anterior surface at the retention portion, a thickness of the device (e.g. as defined between the anterior and posterior surfaces), a shape of the posterior surface. In some embodiments, when on an eye surface, the properties of the retention portion additionally depend on material properties (e.g. flexibility) of the device and the forces which the device experiences when in position on an eye surface and/or underneath an eyelid.

If the anterior surface is planar, in some embodiments, the height of the retention portion is the same as a thickness of the device at the portion.

In some embodiments, heights and/or slope of the retention portion, when in a dry configuration and/or a hydrated configuration, for example, as quantified in this document, are defined as for the device in a relaxed configuration and in relation to a plane on which the device rests, and/or on a plane connecting a majority of an edge circumference of the device.

In some embodiments, heights and/or slope of the retention portion, when in a dry configuration and/or a hydrated configuration, for example, as quantified in this document, are defined as for the device when in a relaxed configuration on a surface with radius of curvature (in one or more directions e.g. a sphere with the radius of curvature) of that expected of the eyeball onto which the device is to be positioned e.g. a radius of curvature of an adult human eyeball, e.g. in both directions, e.g. radius of curvature of about 12 mm.

In some embodiments, heights and/or slope of the retention portion, when in a dry configuration and/or a hydrated configuration, for example, as quantified in this document, are defined as for the device when on an eye surface where, in some embodiments, the device is deformed (e.g. flattened) by suction between the device and the eye surface and/or pressure onto the device (e.g. by the eyelid).

In some embodiments, height of portion/s of the device is affected by curvature of the posterior surface, the higher the curvature the higher the height, for a same thickness device. However, in some embodiments, a higher curvature posterior surface is associated with increased flattening (potentially reducing height/s) of the device when worn on an eye surface.

In some embodiments, the retention portion has a part with a sufficiently high slope followed by a part with a sufficient thickness having a sufficient length, and/or area.

Where, for example, the retention portion has heights with distance from the leading edge (or edge of the retention portion) as described herein and also includes a portion having a height over a holding height, where the portion has a sufficient extent. For example, has an area of at least 1 mm2, or 0.5-3 mm2, or 0.5-2 mm2, or lower or higher or intermediate areas or ranges. Where the holding height is 0.5-5 mm, or 1-3 mm, or lower or higher or intermediate heights or ranges. Where, in some embodiments, the sufficient slope and/or height is followed by the portion having holding height or more e.g. immediately e.g. without a preceding concavity. In some embodiments, a maximum height of the device is less than 3 times, or less than 2 times, or less than 1.5 times the holding height or lower or higher or intermediate multiples or ranges. In some embodiments, the device and/or retention portion, after satisfying slope requirements of the retention portion for 0.5-4 mm has lower slope than that of the retention portion.

In some embodiments, the device retention portion has a height of at least 1 mm at 1 mm inwards from an edge (e.g. leading edge) of the device and/or from an edge of the retention portion. In some embodiments, the device retention portion has a height at 0.5 mm, or at 1 mm, or at 2 mm from a leading edge of the device (and/or from an edge of the retention portion) of at least 50-2500 microns, or at least 500-1000 microns, or lower or higher or intermediate thicknesses or ranges. In some embodiments, the ophthalmic device comprises a section comprising a length of at least 1 mm where a height between said anterior surface and said posterior surface is constant.

Where distance from the leading edge and/or from the edge of the retention portion is measured, in some embodiments, along the anterior surface and/or along a surface as described above, regarding measurement of heights.

In some embodiments, the ophthalmic device, comprises a body comprising a posterior surface which is adjacent to an eye surface when said ophthalmic device is worn, an anterior surface which is adjacent to an eyelid when said ophthalmic device is worn and a retention portion defined by heights between the posterior surface and the anterior surface. In some embodiments, a first height of the heights measured from a first point on the posterior surface to a corresponding point on the anterior surface differs by at least 1 mm, from a second height of the heights measured from a second point on the posterior surface to a corresponding point on the anterior surface, where the second point being distanced at most 1 mm from the first point.

In some embodiments, the ophthalmic device comprises a body that comprises a posterior surface which is adjacent to an eye surface when the ophthalmic device is worn, an anterior surface which is adjacent to an eyelid when the ophthalmic device is worn and a retention portion defined by heights between the posterior surface and the anterior surface. In some embodiments, at least some of the heights between the posterior surface and the anterior surface differ from each other by at least 1 mm when measured from two points on the posterior surface distanced from each other by at most 1 mm.

In some embodiments, the device retention portion is formed, at least partially, once the device is in the eye. For example, in some embodiments, the device crumples and/or squashes and/or folds to provide a retention portion. In some embodiments, under movement of the eyelid.

In some embodiments, the retention portion of the ophthalmic device allows the ophthalmic device to remain between an eyelid and an eye surface. In some embodiments, when the ophthalmic device is positioned under the lower eyelid, the retention portion prevents the ophthalmic device from exiting the lower eyelid. In some embodiments, when the ophthalmic device is positioned under the upper eyelid, the retention portion prevents the ophthalmic device from exiting the upper eyelid. In some embodiments, when the ophthalmic device is positioned on the surface of the eye but not under either the lower eyelid or the upper eyelid (for example positioned in a location on the surface of the eye that is visible), the retention portion of the ophthalmic device prevents the ophthalmic device from sliding under an upper eyelid, while optionally, allowing the ophthalmic device to slide under the lower eyelid when a user closes his eyes.

In some embodiments, the device is thick, for example having a maximal and/or average thickness between the anterior and posterior surface of at least 400-2500 microns, or of at least about 1 mm, or lower or higher or intermediate thicknesses or ranges. A potential advantage of such thicknesses (optionally, in combination with an extent of the retention portion and/or of the device) is a corresponding volume of the device which, in some embodiments, enables loading of the device with therapeutic material. In some embodiments, the device has a volume of 1-500 microliters, or 1-100 microliters, or 1-50 microliters, or 10-50 microliters, or 20-100 microliters, or lower or higher or intermediate volumes or ranges.

In some embodiments, the device has a mass of 1-500 mg, or 1-100 mg, or 1-50 mg, or 10-50 mg, or 10-30 mg, or 15-20 mg, or 20-100 mg, or lower or higher or intermediate masses or ranges.

In some embodiments, the ophthalmic device includes 1-500 mg, or 20-500 mg or 20-150 mg, or 20-120 mg, or lower or higher or intermediate weights or ranges of therapeutic agent/s. In some embodiments, the ophthalmic device is formed of 1% to 50%, or 1-20% or lower or intermediate or higher percentages or ranges of therapeutic agent/s. In some embodiments, an entirety or majority of the ophthalmic device is therapeutic agent/s, for example, the device being at least 80%, or at least 90%, or at least 95%, or at least 99% therapeutic agent/s, or lower or higher or intermediate percentages, by weight and/or volume.

In some embodiments, the ophthalmic device includes large amounts of therapeutic ingredient/s (e.g. about 1 mg to about 20 mg or more than 1 mg, or more than 20 mg, or 0.2-1 mg, or 1-4 mg, or 4-10 mg, or 10-20 mg, or 20-50 mg, or lower or higher or intermediate doses or ranges) and/or is able to elute such ingredient/s to a surface of the eye.

A potential advantage of the ophthalmic device is the ability to dispense therapeutic ingredient, (e.g. a larger amount than that of topically applied preparation/s), to the surface of the eye and/or into the eyeball (potentially replacing eyeball injections). Where for example, a topically applied preparation having up to 1% concentration of therapeutic ingredients applied as eye drops, for example, 30 microliter is, 0.3 mg where a significant portion of the therapeutic ingredient is expected to be flushed naturally out of the eye within a few minutes.

A potential benefit of an ophthalmic device which elutes large amount of medication while residing on an eye surface is the ability to provide and/or maintain adequate concentration of drugs in intranasal regions and/or to the brain and/or to the head and/or to another target organ and/or the ability to provide systemic drug delivery (for example for dosing e.g. “micro dosing” of cannabinoids and/or opioids). In some embodiments the device is used for recreational drug use.

In some embodiments, the retention portion of the device is a portion of the device adjacent to and, in some embodiments, including a leading edge of the device. Where, in some embodiments, the leading edge of a device is defined as a portion of a circumferential edge of the device which, when the device is underneath the eyelid, is nearest to an edge and/or opening of the eyelid.

Without wanting to be bound by theory, in some embodiments, it is theorized that interaction between the tarsal plate of the eyelid with the slope of the device (and/or the retention portion of the device) acts to retain the device within the eyelid, behind the tarsal plate. In some embodiments, interaction between the tarsal plate and the device is associated with deformation of tissue of the eyelid around the device. In some embodiments, alternatively or additionally to force of the tarsal plate, reactive force of the tissue from deformation of the eyelid acts on the device to prevent movement of the device e.g. out of the eyelid and/or deeper into the cul-de-sac of the eye. Alternatively or additionally, in some embodiments, force between the eyeball and a posterior surface of the device acts to prevent movement of the device, where, in some embodiments, the force includes reactive force of pressure of the eyelid onto the device and then to the eyeball and/or suction forces between the device and eyeball.

In some embodiments, the retention portion extends around the ophthalmic device, for example, 20-100% of a perimeter of the ophthalmic device. A potential advantage of a device having a retention portion which extends around (e.g. around a large proportion e.g. more than 50%) of the device is increased retention forces on the device (e.g. of the tarsal plate) and/or in the event of device rotation, increased likelihood that the edge of the eyelid continues to interact with the retention portion.

In some embodiments, the ophthalmic device is symmetrical, for example, having rotational symmetry about an axis. For example about an axis perpendicular to an eye surface on which the device is disposed and/or a central axis e.g. a center axis defined as an axis connecting centers of the anterior and posterior surfaces. A potential advantage of rotational symmetry being maintaining of retention portion interaction with the edge of the eyelid and/or tarsal plate if the ophthalmic device rotates while being worn within the eye.

In some embodiments, the ophthalmic device includes more than one retention portion. A potential advantage being increased area of retention portion/eyelid interaction to hold the device in position. A potential advantage being, if a first retention portion fails and the device escapes from underneath the eyelid and/or the first retention portion escapes from underneath the tarsal plate, the ophthalmic device is potentially retained at least partially underneath the eyelid by a second retention portion. A potential advantage of having more than one raised portion of the device (e.g. as provided by retention portions) is that raised surfaces case pinch-removal. Pinch-removal being, in some embodiments, where two opposing forces are applied on different portions of the device to break suction between the device and the eye surface and/or push portion/s (e.g. between the portions to which the forces are applied) of device away from the eye surface. Where, in some embodiments, the opposing forces are applied manually by a user e.g. by a thumb and other finger e.g. index finger. for example, is provided at outer region/s along an axis of elongation of the device In some embodiments, an ophthalmic device including more than one retention portion includes one or more cavity on a posterior surface of the device e.g. a cavity for each protrusion.

In some embodiments, for example, where the device is symmetrical, the retention portion is formed by entire edge of the device or a large proportion e.g. at least 50%, or at least 80% or at least 90% of the perimeter of the device. A potential benefit of all or a large proportion of edges of the device being increased likelihood of retention of the device underneath the eyelid in the circumstance of rotation of the device.

In some embodiments, the ophthalmic device is configured to adhere to an eye surface onto which it is positioned. In some embodiments, if the device escapes from underneath the eyelid, partially or fully, adhesion forces of the device to the eye surface (e.g. sclera) are sufficient to maintain the device on the eye e.g. for at least a short period of time e.g. during blinking (in some embodiments, during a few blinks e.g. 1-10). Where, in some embodiments, upon escape and/or emergence of the device from underneath the eyelid, the device is returned (e.g. manually) to underneath the eyelid.

In some embodiments, adherence of the ophthalmic device to an eye surface is sufficient to prevent rotation of the device. For example, for devices which are not rotationally symmetrical and/or for devices which have a localized retention portion and/or a retention portion disposed asymmetrically on the device.

In some embodiments, adherence of the ophthalmic device to an eye surface (e.g. sclera) is associated with suction forces between the device and the eye surface.

For example, in some embodiments, a posterior surface of the ophthalmic device adjacent to the eye surface is curved, e.g. is concave, in one or more direction. The curve potentially increasing suction forces between the device and the eye surface.

In some embodiments, a curvature of the posterior surface is 0.1-1 that of the sclera, in one or more direction. In some embodiments, a radius of curvature of the posterior surface, in one or more direction, is 1-15 mm, or 1-10 mm, or lower or higher or intermediate radii or ranges and/or has a ratio to that of the sclera being 0.1-2, or 0.5-1, or lower or higher or intermediate ratios or ranges. In some embodiments, a lower curvature of the posterior surface is associated reduced suction forces between the eye surface, potentially enabling re-positioning and/or increasing comfort to a user.

In some embodiments, a curvature of the posterior surface, in one or more direction, is lower than that of the sclera, for example a ratio of the radius of curvature of the posterior surface to that of the sclera being greater than 0.8. In some embodiments, different portions of the posterior surface have different radii of curvature, but, in some embodiments, each of these have a curvature lower than that of the sclera.

In some embodiments, dimensions and/or ratios thereof (e.g. curvatures) as described in this document relate to average adult human eye curvatures. Where, for example, in some embodiments a radius of curvature of the sclera, RS is about 12 mm. In some embodiments, dimensions and/or curvatures are selected for different anatomies, for example for a child's eye, for example for an animal eye, for example for a different surface (e.g. mucosal surface).

Where, for example, when the device is used in an eye having different dimensions and/or curvatures (e.g. animal eye, child's eye) size/s and/or curvature/s of the device are scaled for use in the eye type and/or in a specific eye (e.g. personally tailored devices). For example, where one or more dimension of the device e.g. length and/or width of the device footprint and/or retention region height and/or slope (e.g. as described within this document) is scaled, for example, using one or more measurement parameter of the eye in question e.g. a radius and/or diameter and/or largest extent of the eye.

In some embodiments, for example, where the surface includes cavity/ies and/or protrusion/s and/or has a surface texture, radius of curvature of a surface (and/or edge and/or corner) is defined as a radius of a circle which matches curvature of at least 60%, or 80% or 90% of the surface (and/or edge and/or corner).

In some embodiments, the device remains has a posterior surface with low radius of curvature. In some embodiments, the device remains in position despite having a posterior with low radius of curvature. For example, where a ratio between the radius of curvature of the posterior surface to the radius of curvature of the eye is 0.1-0.8, or 0.1-0.5, or 0.6-0.95, or 0.8-0.95, or 0.8-1, or larger than about 0.8, or lower or higher or intermediate ranges or ratios. For example, where the radius of curvature of the posterior surface is the same as or larger than that of the portion of the eye to which the device is to be adhered e.g. the sclera. For example, in some embodiments, despite having a posterior surface which is flat or convex. For example, where one or more of mucoadhesive property/ies and/or anchoring feature/s enable the device to adhere to the eye surface.

In some embodiments, the ophthalmic device includes multiple layers and/or portions with different material properties. In some embodiments, the device includes one or more of a lubricous layer, a mucoadhesive layer and an additional layer. In some embodiments, one or more layer includes therapeutic material.

In some embodiments, a body of an ophthalmic device has a concave posterior surface. In some embodiments, concavity of a posterior surface in contact with the eye surface (e.g. sclera), increases adhesion of the device to the eye surface. The concavity, for example, providing suction force between the device and eye surface, adhering the device to the eye surface. Optionally, in some embodiments, a radius of curvature of one or more portion of the posterior surface of the device is smaller than that of the portion of the eye to which the device is to be adhered to. For example, the sclera.

In some embodiments, the body of the ophthalmic device is not hollow. In some embodiments, the body of the ophthalmic device is made of a continuous material, meaning it does not comprise macroscopic gaps and/or empty areas within it. In some embodiments, a gap or empty area is defined as macroscopic if it is larger than 0.2 mm.

In some embodiments, the body of the ophthalmic device has an oval or round form. In some embodiments, the body of the ophthalmic device is symmetrical relative both to its longitudinal axis and relative to its transversal axis. For example, when looking at the ophthalmic device from the anterior surface, the device is symmetrical relative to its longitudinal axis and relative to its transversal axis. In some embodiments, the device comprises an elliptical and/or oval cross-sectional shape.

In some embodiments the posterior surface is smooth for example, potentially reducing irritation associated with contact between the eye surface and the device. In some embodiments, the posterior surface includes rough portion/s and or anchor/s potentially increasing adhesion by friction between the posterior surface and the eye surface. In some embodiments, edge regions (e.g. and optionally, in some embodiments, not a central region which is smooth) of the posterior surface are rough and/or include anchor/s and/or protrusions and/or cavities. In some embodiments, the posterior surface is smoother than the anterior surface. In some embodiments, both the posterior surface and the anterior surface are similarly smooth.

In some embodiments, a device includes one or more feature to increase adherence between the device and the sclera. For example, roughness and/or anchors (e.g. cavity/ies, protrusion/s and/or hook/s) for example, located at a posterior surface and/or at edges of the device.

An aspect of some embodiments relates to an ophthalmic device configured to remain underneath an eyelid (for example, having a retention portion) but that has mobility and/or allows fluid flow between the device and an eyeball surface. A potential advantage of mobility or fluid flow between the device and the eyeball being increased user comfort and/or reduced risk of bacterial growth underneath the device (e.g. associated with tear fluid exchange).

In some embodiments, mobility of the device on the eye surface is associated with low suction between and/or an easily breakable suction seal between a posterior surface of the device and the eyeball surface. For example, in some embodiments, the posterior surface includes one or more portion which is has low concavity (e.g. with radius of curvature as described above), or is not concave, or is convex, potentially reducing the ability of the device to seal with the eyeball surface. In some embodiments, the device is able to form a seal with the eyeball surface, for example, having convex posterior surface and/or flexible edges where the seal is easily broken e.g. to allow the device to move on the eye surface (e.g. periodically). For example, in some embodiments, the device includes a sealing edge (e.g. flexible) only on a portion of the device. For example, in some embodiments, the device is elongate, potentially casing breaking of suction forces between the device and the eye surface.

An aspect of some embodiments of the invention relates to an ophthalmic device which maintains size and/or shape both in storage and in use where, in some embodiments, the device is provided in a dry form and optionally hydrates during residency on an eye surface. A potential benefit being reduced distortion of the device during hydration and/or use and/or maintained shape and/or maintained mechanical characteristics of the device during use. For example, while being able to store the device in a dry form where therapeutic agent/s are less likely to be degraded and/or degrade less rapidly e.g. potentially increasing shelf life of the device.

In some embodiments, as the device hydrates (e.g. on an eye surface) the device increases in volume by less than 20%, or less than 15%, or less than 10%, or less than 5%, or lower or higher or intermediate percentages. In some embodiments, one or more maximal cross sectional dimension as the device hydrates increases by less than 20%, or less than 15%, or less than 10%, or less than 5% or lower or higher or intermediate percentages.

An aspect of some embodiments of the invention relates to an ophthalmic device which, when dry and when hydrated is soft. Where, in some embodiments, the device is provided in a dry form and optionally hydrates during residency on an eye surface. A potential advantage of a soft device in dry form, is enabling application of the device to the eye without prior hydration and/or softening.

A potential benefit of a soft ophthalmic device is reduced irritation to the eye e.g. reducing tearing associated with presence of the device which, in some cases, flushes therapeutic material and/or nutrients.

In some embodiments, at least a portion of the ophthalmic device degrades and/or disintegrates within the eye. In some embodiments, the ophthalmic device is a biocompatible device which is designed to be partially or completely biodegradable and/or biocrodible.

For example, in some embodiments, a portion of a device degrades e.g. once the device is adhered to the eye surface. For example one or more mucoadhesive portion and/or one or more lubricous portion.

For example, in some embodiments, one or more portion of the device is selected to degrade within the eye, in about 1 minute to 30 minutes or 15 minutes to 1 hour or about 0.5 hour to 8 hours, or 4 to 24 hours, or 12 hours to 3 days, or 1 day to 7 days, or 3 days to 2 weeks, or 1 week to 1 month, or lower or higher or intermediate durations or ranges.

In some embodiments, one or more portion degrades to enable eluting of therapeutic material. For example, in some embodiments, as a portion comprising therapeutic material degrades, it elutes the therapeutic material into eye tissue and/or eye fluid. For example, in some embodiments, a portion degrades to reveal a portion comprising therapeutic material. Where, in some embodiments, the degrading portion initially covers at least a portion of the portion comprising therapeutic material.

In some embodiments, at least a portion of the device is degraded and/or its properties are deteriorated when exposed to a biological environment, for example, a biological system e.g. the eye, and/or to a similar in vitro environment simulating conditions of a biological system. In some embodiments, degradation and/or deterioration of the device is manifested by change (e.g. reduction) in one or more of device's physical properties, for example one or more of; device integrity, tensile strength and elasticity of the device body (e.g. of film of the device body).

In some embodiments, degradation occur from the posterior surface towards the anterior surface, for example, the device maintaining a shape of the anterior surface, for a portion of time during which the device is degrading. In some embodiments, degradation from the posterior surface is more rapid than that of the anterior surface. In some embodiments, the device includes different portions which degrade at different rates. In some embodiments, lower speed degrading and/or non-degrading portions acting to maintain a shape (e.g. retention portion shape) for a time period.

In some embodiments, the device is designed to break into portions, for example, upon application of manual pressure. Where, in some embodiments, a size and/or shape and/or brittleness of the device is selected to provide this feature.

An aspect of some embodiments of the invention relates to an ophthalmic device having one or more retention portion, and including a thin and/or conforming edge portion.

In some embodiments, for example, where the device includes a thin edge portion and/or portion extending from a body of the device, the retention portion is defined as a region of the device fulfilling the requirements of height and/or slope e.g. as described above.

In some embodiments, the device (e.g. curvature of one or more portion of the device e.g. to generate sufficient suction to hold the device in position) conforms to an eye. Where, for example, in some embodiments, an edge portion is sufficiently thin and/or flexible to conform to a surface of the eye.

In some embodiments, the device has thin edges and/or edges of the device recessed into a surface of the eye. In some embodiments, moving from a leading edge of the device, the device includes a thin edge followed by a retention portion. In some embodiments, a step formed between an edge of the device and the eye surface, for example, by the thin edge next to the retention portion, is 1-100 microns, or 10-50 microns, or lower or higher or intermediate ranges or steps.

In some embodiments, edge characteristics (e.g. shape and/or thickness) as described in this document are for all of or a portion of an edge region of the device. For example, a portion of a circumferential edge of the device. For example, for 20-99%, or 80-90%, or lower or higher or intermediate ranges or percentages, of a circumference of the device. In some embodiments, different portions of a circumferential edge of a device have different properties e.g. shape and/or thickness.

In some embodiments, adherence to the sclera is associated with adherence of edges of the device to the sclera, for example influenced by an extent of conforming of the device to the sclera and/or of the sclera to the device.

In some embodiments, flexibility and/or elasticity of the device increases sealing between the device edge and the eye surface. In some embodiments, the device includes one or more edge portion which is flexible and/or elastic e.g. sufficiently thin to be flexible.

In some embodiments, the device has an edge designed for user comfort and/or to increase adherence to the eyeball. In some embodiments, the ophthalmic device has a thin edge. In some embodiments, an edge (e.g. circumferential edge) of the device has average thickness of about 15 microns, or about 20 microns, or about 30 microns or about 50 microns. In some embodiments, the ophthalmic device has a thin edge of about 15 microns, or about 20 microns. In some embodiments, the ophthalmic device has an edge thickness of about 5-200 microns, or 5-200 microns, or lower or higher or intermediate thicknesses or ranges.

In some embodiments, for example, alternatively to having a thin edge. Or where, a device, in some embodiments, has an edge circumference where only a portion of the circumference is thin, in some embodiments, the edge of the device is blunt and/or curved and/or rounded for example, increasing comfort of wear for a user.

In some embodiments, the edge of the device is sharp e.g. chisel shaped and/or knife edged.

In some embodiments, the edge of the ophthalmic device is defined as a 0.1-1 mm edge region, or 0.1-0.5 mm edge region of the device, or lower or higher or intermediate distances or ranges. In some embodiments, an edge thickness is measured at 0.1 mm, or 0.5 mm, from a circumference of the device.

In some embodiments, a thin edged device provides improved sealing between edges of the device and the eye surface. A potential advantage of sealing between the device edges and the eye surface is increased suction between the device and the eye surface. A potential advantage of scaling between the device edges and the eye surface is reduced accumulation of debris (e.g. one or more protein, mucus, oil, and skin cells) of between the device and the eye surface potentially minimizing risk of infection associated with use of the ophthalmic device.

In some embodiments, a thin edged device conforms to a shape of the eye surface at the device edges.

In some embodiments, an edges of the device provide anchoring of the device into the eye surface e.g. soft tissue of the conjunctiva e.g. the soft and thick scleral conjunctiva which is apparently, about 25-40 microns thick and is apparently even thicker in the fornix. In some embodiments, the device recesses into the eye surface at edges of the device and/or makes an indentation in the eye surface, for example, the recessing associated with one or more of pressure between edges of the device and/or softness of the eye surface.

In some embodiments, portion/s of the eye are deformed in depth (e.g. by the device), for example at edges of the device (and/or at region/s adjacent to protrusions and/or cavities of the device) e.g. by 50 microns to 500 microns, or lower or higher or intermediate dimensions or ranges. In some embodiments, tissue local to edges of the device deforms, for example, a region of tissue at most 0.5 mm, or 1 mm or lower or higher or intermediate widths of tissue at a device edge.

In some embodiments, edges (and/or edge region/s) of a device conform to a shape of the eye surface. For example, in some embodiments, edge region/s are flexible and/or clastic and/or sufficiently thin to conform to the eye surface. In some embodiments, a device body is flexible and/or elastic e.g. sufficiently flexible and/or elastic to conform to the eye surface, at least partially. Potentially, device body and/or edges conforming to the eye surface improves user comfort and/or reduces risk of dislodgement of the device.

In some embodiments, the posterior surface includes one or more cavity or protrusion, for example, on the posterior surface of the device.

Without wanting to be limited by theory, it is hypothesized that, in some embodiments, a cavity increases suction between the device and the eye surface e.g. localized suction at the cavity. In some embodiments, eye tissue enters the cavity (e.g. under suction forces of the cavity), potentially increasing adherence between the device and the eye. A potential advantage of cavity/ies on the posterior surface is extended residence time of the device on the eye.

In some embodiments, the device has a plurality of cavities, where in some embodiments, two or more of the cavities are about the same size and/or shape. Where, in some embodiments, two or more of the cavities are different from each other, for example, in size and/or shape.

In some embodiments, an opening size of a cavity, is between 0.005 mm and 20 mm, or 0.005-2 mm, or 0.005-1 mm, or 0.005-0.5 mm, or 1-2 mm, 1-4 mm, or 2-6 mm 3-8 mm, or 4-10 mm, or 5-20 mm or lower or higher or intermediate ranges or dimensions. In some embodiments, a cavity has a large opening, for example, extending over at least half of an area of the surface (e.g. posterior surface) and/or having an extent of 1-10 mm, or 1-8 mm, or 1-5 mm, or 5-20 mm or lower or higher or intermediate extents or ranges. In some embodiments, an aperture of a cavity is macroscopic or microscopic in size. In some embodiments, a depth of a cavity is 5 microns-2 mm, or 5 microns-1 mm, or 5-400 microns, or 5-200 microns, 200-600 microns 400-800 microns 600-1500 microns 800-2000 microns or lower or higher or intermediate depths or ranges. In an exemplary embodiment, a depth of a cavity is 400-1200 microns, or lower or higher or intermediate depths and ranges. In some embodiments, a cavity extends through a proportion of a thickness of the device, for example, through at 10-20% 20-30% 30-50% 50-90%, or 80-99%, or 90-99%, 50-70%, 60-90%, or lower or higher or intermediate ranges or percentages of a thickness of the device in a region of the cavity.

In some embodiments, the ophthalmic device includes one or more protrusion from one or both surfaces of the device.

In some embodiments, protrusion/s have a rounded and/or blunt shape.

In some embodiments, the device has a plurality of protrusions, where in some embodiments, two or more of the protrusions are about the same size and/or shape. Where, in some embodiments, two or more of the protrusions are different from each other, for example, in size and/or shape.

In some embodiments, a size of a protrusion, above a surface (e.g. posterior and/or anterior) of the device is 5-400 microns, or 20-200 microns, or 200-800 microns or 400-1200 microns or 600-1500 microns lower or higher or intermediate ranges or sizes. In some embodiments, an extent of a footprint of a protrusion on the surface of the device is 5 microns to 3 mm, or 5 microns to 1 mm, or 5-700 microns, or 5-400 microns or 3-6 mm, or 4-8 mm, or 4-10 mm, or 6-12 mm or lower or higher or intermediate extents or ranges.

A potential benefit of protrusion/s is increased adherence of the ophthalmic device to the eye surface. In some embodiments, protrusion/s increase friction between the eye surface and the eye surface. In some embodiments, protrusion/s increase (e.g. between the protrusions) increase the suction force between the device and the eye surface.

In some embodiments, the ophthalmic device includes mucoadhesive material e.g. on the posterior surface of the device which potentially increases adhesion between the device and the eye surface on which the device is positioned.

Optionally, in some embodiments, the posterior surface includes one or more mucoadhesive part. Where mucoadhesive material/s in some embodiments, increase adhesion of the device to the eye surface. In some embodiments, the posterior surface includes a mucoadhesive layer. Where, in some embodiments, the layer covers the posterior surface. Alternatively, in some embodiments, the mucoadhesive layer is a non-continuous surface, where hole/s, for example, allow water penetration and/or drug transportation e.g. to a layer underneath the mucoadhesive layer. In some embodiments, the mucoadhesive layer has a ring-shape including an aperture in the center e.g. enabling water penetration and/or drug transportation.

The mucoadhesive layer in the device, in some embodiments, is provided dry and/or semi-hydrated. Where, in some embodiments, upon application of the device to the eyeball, the mucoadhesive layer hydrates and becomes sticky, for example, in less than 10 seconds, or in less than 30 seconds, or in less than 1 minute.

In some embodiments, the material/compound/polymer in a mucoadhesive layer has an adherence force of 80-200%, or of 100-200%, or of over 100%. Where, for example, pectin is defined as having an adherance force of 100%.

In some embodiments, mucoadhesive portions include at least one mucoadhesive compound, which in some embodiments, is selected from the group consisting of gelatin, alginates, chitosan, amylose, collagen, sodium poly-acrylate, modified starch, elastin, polyacrylic acid and combinations thereof.

In some embodiments, the device presents a low friction surface to the eyelid (e.g. is smooth). A potential advantage of which is reduced likelihood of dislodgement of the device. In some embodiments, the device presents a low friction surface to the eye surface (e.g. is smooth). A potential advantage of which is reduced likelihood of discomfort to the user. In some embodiments, the device presents a same low level of friction to the eyelid and to the eye surface.

Optionally, in some embodiments, one or more of portion of an anterior surface of the device is smooth and/or includes lubricious material. Where the anterior surface of the device is a surface which is in contact with the inner surface of the eyelid, at least part of the time. Potentially, in some embodiments, smooth and/or lubricous material reduces friction between the eyelid and the surface. Potentially, in some embodiments, the smooth and/or lubricous material minimizes irritation of the eye, e.g. the eyelid, e.g. associated with the ophthalmic device. In some embodiments, the posterior surface is smoother than the anterior surface. In some embodiments, both the posterior surface and the anterior surface are smooth.

In some embodiments, a lubricious material layer covers the anterior surface where, in some embodiments, the layer is continuous. In some embodiments, the lubricious layer is a non-continuous surface, (e.g. including one or more hole) for example to allowing water penetration and/or drug transportation. For example, in some embodiments, a lubricious material layer includes holes. In some embodiments, the anterior surface hosts one or more lubricious material portion where, in some embodiments, one or more of the portions is connected to other portion/s and/or one or more of the portions is not connected to other portions. Optionally, in some embodiments, the lubricous surface is temporary, where, in some embodiments, one or more portion of lubricous material degrades and/or disappears (e.g. is absorbed and/or expelled by the eye), for example, before other portion/s of the device degrade.

In some embodiments, a material layer has variable thickness and/or variable material properties e.g. a lubricious layer and/or a mucoadhesive layer. Where, for example, upon attachment to the eye, a material has a larger extent on the anterior surface than after a time period of residence on the eye, where, in some embodiments, more rapidly degraded portion/s (e.g. thinner portions and/or portions having more a rapidly degraded composition) of the layer dissolve prior to other portion/s e.g. to reveal lower layers which in some embodiments comprise therapeutic agent/s.

An aspect of some embodiments of the invention relates to a kit comprising an ophthalmic device (e.g. as described in this document) and instructions for use thereof. In some embodiments, the kit of the comprises means for delivering the device into the eye of a patient. For example, an applicator. In some embodiments, the ophthalmic device is packaged together with the applicator.

Optionally, in some embodiments, the kit includes a degradation formulation which, when contacted with the device when the device is on an eye surface of a patient, structurally degrades the device and/or accelerates degradation of the device.

In some embodiments, devices are provided in multi-device packs. Where, in some embodiments, two or more devices are interconnected to one another, or separated.

In some embodiments, the ophthalmic device is one or more of flexible, elastic, and supple e.g. to conform with a shape of the eyeball. In some embodiments, the ophthalmic device elasticity and/or softness is similar to the elasticity and/or softness of soft contact lenses. In some embodiments, the ophthalmic device has elasticity or softness, in one or more direction, with a Young's modulus of 0.01-1.5 MPa or 0.05-0.15-1.5 MPa, or 0.25-0.35 MPa or lower or higher or intermediate elasticities/softness or ranges. In some embodiments, the ophthalmic device has elasticity, in one or more direction, with a Young's modulus of 0.01-200 MPa. In some embodiments, the ophthalmic device has elasticity, in one or more direction, (e.g. Young's modulus 0.01-200 MPa, or 0.05-0.15 MPa or 0.15-0.35 MPa, or lower or higher or intermediate elasticities or ranges) in a dry form. In some embodiments, the ophthalmic device has elasticity, in one or more direction, (e.g. Young's modulus 0.01-200 MPa, or 0.05-0.15 MPa, or 0.01-0.35 MPa, or lower or higher or intermediate elasticities or ranges) in a wet form while in the eye. In some embodiments, the ophthalmic device has elasticity allowing elongation of 10% to 800% or more (in dry form and/or in wet form).

In some embodiments, the ophthalmic device is one or more of rigid, and shaped e.g. shaped to conform with a shape of the eyeball. In some embodiments, the ophthalmic device elasticity and/or hardness, in one or more direction, similar to the elasticity and/or hardness of hard or rigid contact lenses. In some embodiments, the ophthalmic device has elasticity, in one or more direction, with a Young's modulus of 0.1-200 MPa, or lower or higher or intermediate ranges or elasticities. In some embodiments, the ophthalmic device has this elasticity, in one or more direction, when in a dry form. In some embodiments, the ophthalmic device has this elasticity in the wet form e.g. while in the eye.

In some embodiments, the ophthalmic device has layers with different material properties e.g. elasticity and/or flexibility. In some embodiments, the ophthalmic device has regions having different material properties e.g. elasticity and/or flexibility.

In some embodiments, the device is flexible, potentially enabling the device to shape to a surface of the eye e.g. potentially increasing user comfort (e.g. allowing the eyelid to move smoothly above the device with little or no interference and/or discomfort) and/or reducing dislodgement forces experienced by the device. In some embodiments, a flexible device has a concave posterior surface, where suction (e.g. associated with interaction between concavity of the posterior surface and the eye surface) between the device and the eye surface then flattens the device onto the eye surface.

In some embodiments, elasticity of the device increases suction force between the device and the eye surface to adhere the device to the eye. Where, for example, in some embodiments, elasticity compensates for low concavity curvature of the posterior surface of the device.

In some embodiments, flexibility and/or elasticity of the device increases sealing between the device edge and the eye surface.

In some embodiments, one or more portion is configured to fit an eye surface and/or a portion of an eye surface and/or an orifice. In some embodiments, the device is selected to be compatible with size of the inferior or superior conjunctival sac where the device deforms the sac only slightly, for example, by at most 2 mm for example, by only an amount which is comfortable for a user and/or does not cause harm to the eye surface and/or eyelid and/or other tissue/s. For example, in some embodiments, only the retention portion deforms the sac. It has been found that devices with a height of 2 mm can be comfortable and, in some embodiments, up to a height of 4-6 mm. It has been found that, in some embodiments, a device width of 5-10 mm is comfortable.

Where anatomical sizes, in some embodiments, are defined using one or more of the below references which are hereby incorporated by reference in their entirety:
www(dot)pubmed(dot)ncbi(dot)nlm(dot)nih(dot)gov/9019385/
www(dot)onlinelibrary(dot)wiley(dot)com/doi/abs/10(dot)1111/j(dot)1755-3768(dot)1965(dot)tb06387(dot)x
www(dot)pubmed(dot)ncbi(dot)nlm(dot)nih(dot)gov/15167728/

In some embodiments, the ophthalmic device has a thickness of less than 3 mm, or less than 2.5 mm, or less than 2 mm, or less than 1.5 mm, or less than 1 mm, or less than 0.8 mm, or less than 0.6 mm, or less than 0.5 mm, or less than 0.4 mm, or less than 0.3 mm or lower or higher or intermediate thicknesses.

In some embodiments, the ophthalmic device has a thickness of greater than 3 mm, or greater than 2.5 mm, or greater than 2 mm, or greater than 1.5 mm, or greater than 1 mm, or greater than 0.8 mm, or greater than 0.6 mm, or greater than 0.5 mm, or greater than 0.4 mm, or greater than 0.3 mm, or lower or higher or intermediate thicknesses or ranges.

In some embodiments, pressure between the eye and the device is increased by increasing a thickness of the device and/or a thickness of an edge region of the device. Where, increased pressure (and/or sealing between the eye and the device e.g. associated with pressure and/or contouring of the eye to the device or the device to the eye) in some embodiments, reduces tear fluid penetrations between the device and the eye surface.

In some embodiments, the device is small e.g. sufficiently small that it fits within a space underneath an eyelid (e.g. entirely). In some embodiments, a maximum and/or average extent of a device is about 4-12 mm, or 4-10 mm, or 4-8 mm, or 3-8 mm, 1-8 mm, or 4-6 mm, or lower or higher or intermediate ranges or sizes. In some embodiments, the ophthalmic device has a size (e.g. maximum and/or average extent) of about 4 mm. In other embodiments, the device has dimensions of between about 0.5 to about 20 mm in maximum size. A potential advantage a small dimensioned device is increased user comfort and/or lower likelihood of dislodgement of the device during movement of an eyelid over the device.

In some embodiments, an ophthalmic device is elongated. Where, for example, in some embodiments, a dimension of the device measured along an axis of the elongation is about 0.5-20 mm.

Where, in some embodiments, an extent of the device is measured as a maximum dimension of a smallest cuboid shape into which the device fits. In some embodiments, for example, associated with low curvatures of the device surfaces, the cuboid bounding shape is thinner in one dimension, (e.g. associated with a direction into the eye when the device is on the eye) than in the other two dimensions. In some embodiments, the body of the ophthalmic device is not hollow. In some embodiments, the body of the ophthalmic device is made of a continuous material, meaning it does not comprise macroscopic gaps and/or empty areas within it. In some embodiments, a gap or empty area is defined as macroscopic if it is larger than 0.2 mm.

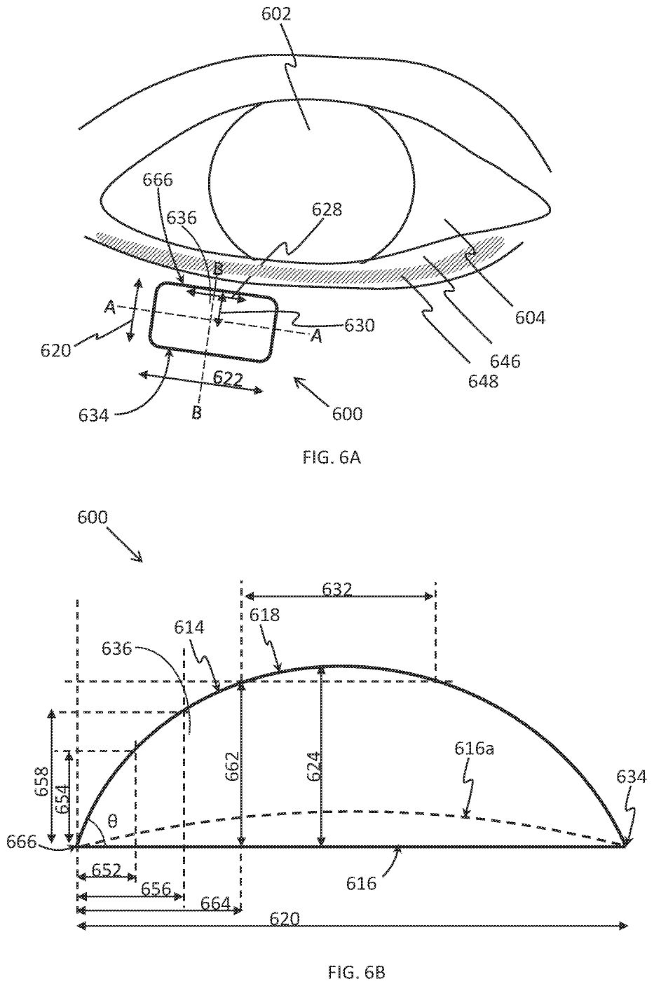

In some embodiments, as shown for example in FIG. 6A, the body of the ophthalmic device is symmetrical relative to its longitudinal axis (schematically shown by line AA) and relative to its transversal axis (schematically shown by line BB). For example, when looking at the ophthalmic device 600 from the anterior surface, the device is symmetrical relative to its longitudinal axis (AA) and relative to its transversal axis (BB). In some embodiments, for example, in the embodiment schematically illustrated in FIG. 6A, the symmetries in relation to both axes are mirror reflection symmetry. In some embodiments, the device comprises an elliptical and/or oval cross-sectional shape.

A potential advantage of the ophthalmic device is convenience of use where, in some embodiments, the device is applied by the user once to cover a time period longer than of topically applied preparations, for example, a time period of about a minute or longer than a minute, or longer than 10 minutes, or longer than an hour, or longer than a day. Where, in some embodiments, the device serves as controlled release vehicle for one or more therapeutic ingredient.

A potential benefit of an ophthalmic device configured to reside on an eye surface is potentially increased residence time of a treatment drug in the eye. For example, as compared to periodically applied topical (e.g. eye drops, ointment).

A potential benefit of an ophthalmic device which elutes medication while residing on an eye surface is the ability to provide and/or maintain adequate concentration of drugs in the pre-corneal/pre-scleral tear film e.g. for extended periods of time.

A potential benefit of an ophthalmic device which elutes medication while residing on an eye surface e.g. maintaining a position on the eye surface is the potential ability for medication to diffuse through eye tissue. For example, penetrating into the sclera and/or into the cornea and/or into the eyeball, and/or reaching more internal portion/s of the eye. Potentially enabling, in some embodiments, topical medication of those areas which are usually treated by injection e.g. into the eyeball.

A potential benefit of an ophthalmic device (e.g. with extended residence and/or elution times is) is the ability to provide and/or maintaining adequate concentration of drugs to intranasal region/s (e.g. as delivered by tear fluid) and/or to the brain and/or to the head and/or to another target organ and/or as systemic drug delivery.

In some embodiments, the ophthalmic device being in residence for a period of time enables steady elution of treatment materials into the eye potentially reducing effects of periodic dosing such as, for example, a transient period of systemic and/or local overdose (potentially associated with high risk of side effects) e.g. followed by an extended period of sub-therapeutic levels before the administration of next dose. Where the rate of tissue drug uptake is high at early times after dosing, but declines rapidly. A long residence time, for example, as compared to, eye drops applied to the eye e.g. the surface of the eyeball, where blinking and natural tear flow combine to limit time that liquid medicament will remain effective e.g. to a few minutes. Potentially, less therapeutic ingredient per treatment is used in treatment of a subject using an ophthalmic device e.g. as opposed to topical preparations where therapeutic ingredients may be washed out before they are effective requiring higher quantities of therapeutic ingredient and/or where high patient compliance is more likely to be lower as application is frequent potentially resulting in more discarded un-used therapeutic ingredients.

In some embodiments, the ophthalmic device protects a portion of the eye. For example, from external impact/s and/or chemical changes. For example, to ameliorate and/or reduce ocular damage upon orbisculation (citrus fruit squirting juice into the eye). In some embodiments, the ophthalmic device protects a portion of the eye, enabling the portion to heal. For example, in some embodiments, an incision and/or wound in the eye is closed and/or covered by placing the ophthalmic device over the incision. Where, suction forces in between the eye surface and the device, potentially assisting in closing and/or maintaining closure of the incision and/or wound potentially hastening healing.

In some embodiments, a device is tailored to an individual subject, where, for example, in some embodiments, one or more of size, shape, curvature of surface/s, type of therapeutics, release time, residence time are selected for the individual and/or according to an individual treatment plan.

Although throughout this document, ophthalmic use of the device is described, it should be understood that one or more embodiments and/or combinations of embodiments of the device as described within this document, in some embodiments, are used in treatment of other portions of a human or animal body. For example, for portions of the eye of the than the sclera, e.g. the cornea, the cul-de-sac of the eye. For example, other mucosal surface/s e.g. the inner mouth e.g. the intranasal surface. For example, other tissue types and/or organs.

Although throughout this document, ophthalmic use of the device is described, it should be understood that one or more embodiments and/or combinations of embodiments of the device as described within this document, in some embodiments, are used for other applications such as: to host e.g. contain, have attached to and/or provide a support for one or more type of electronics e.g. on the eye. For example, one or more active electronic element and/or passive modules and/or components e.g. an antenna, RFID. For example, one or more capacitor and/or resistor and/or, shape memory element, or may act as a support for other element or elements in the ocular region etc.) Where, for example, in some embodiments, electronics include on or more of sensor/s (e.g. temperature sensitive element, pressure sensitive element, humidity sensitive element), imager/s (e.g. camera), a user interface, a processor, transmitter, receiver, illumination element, led screen, power source. In some embodiments, electronics (e.g. using sensor/s) are used for therapeutics (e.g. Electroconvulsive therapy (ECT) treatment) and/or diagnostics. In some embodiments, one or more electronic part is used for entertainment (e.g. VR) and/or aesthetic enhancement. In some embodiments, the device is used for entertainment and/or for aesthetic enhancement.

In some embodiments, more than one device as described in this document is used to treat a subject, for example, where multiple devices are in situ on an eye surface at the same time, for example, each device providing a particular dose. Where, in some embodiments, to provide a higher dose more than one device is applied. In some embodiments, multiple devices as described in this document are provided packaged together and/or where the multiple devices are provided connected.

In some embodiments, the device includes and/or is constructed from non-toxic and/or biocompatible materials. In some embodiments, where therapeutic material is toxic in high concentrations, the entire device, in some embodiments, contains less than a toxic dose and/or the device elutes the therapeutic material at sub-toxic levels.

In some embodiments one or more portion of the device include dye and/or pigment e.g. for case of identification while in the eye, and/or during insertion and/or extraction, and/or during production. In some embodiments, dye and/or color is printed into one or more identifying shape and/or letter and/or numeral and/or barcode. In some embodiments, one or more identifier is embossed or stamped on the device. In some embodiments, the device includes one or more electronic identifier e.g. RFID on or within a body of the device. In some embodiments, device dye and/or pigment includes one or more of blue, red, fluorescent yellow, and/or any other color.

In some embodiments, the ophthalmic device is manufactured from a film, for example, a multi-layer film. Where, in some embodiments, the film is cut into shape to form individual devices. In some embodiments, the film and/or individual devices are shaped for example, to form desired curvatures of surfaces. Where, in some embodiments, shaping is by casting of a solution onto a shaped support surface e.g. a mold. Where, in some embodiments, shaping is by application of mechanical and/or chemical treatment e.g. pressure and/or heat. In some embodiments, therapeutic ingredients are incorporated into one or more solution each of which is successively cast onto a support surface to provide a multi-layer film. In some embodiments, separately cast layers have one or more different properties (e.g. thickness, mucoadhesive, lubricious, containing therapeutic agent/s). In some embodiments, one or more surface is treated to provide it with different properties e.g. a surface is treated to become more adhesive and/or more lubricious.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Methods

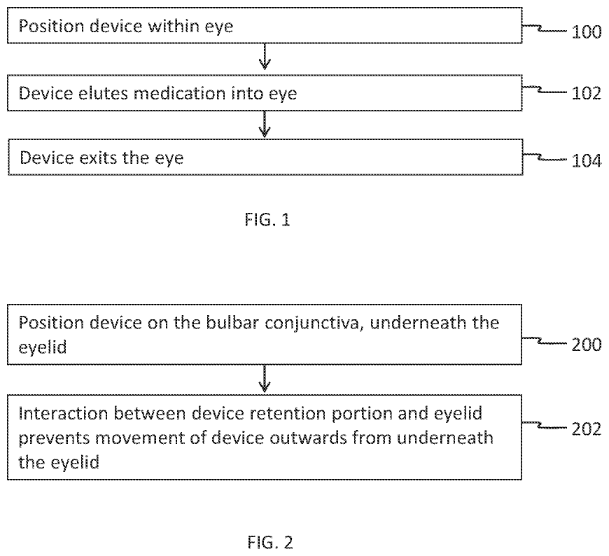

FIG. 1 is a method of use of an ophthalmic device, according to some embodiments of the invention.

At 100, in some embodiments, the ophthalmic device is positioned within an eye. For example, place on an eye surface, for example, onto a portion of the sclera.

At 102, in some embodiments, the ophthalmic device elutes medication into the eye.

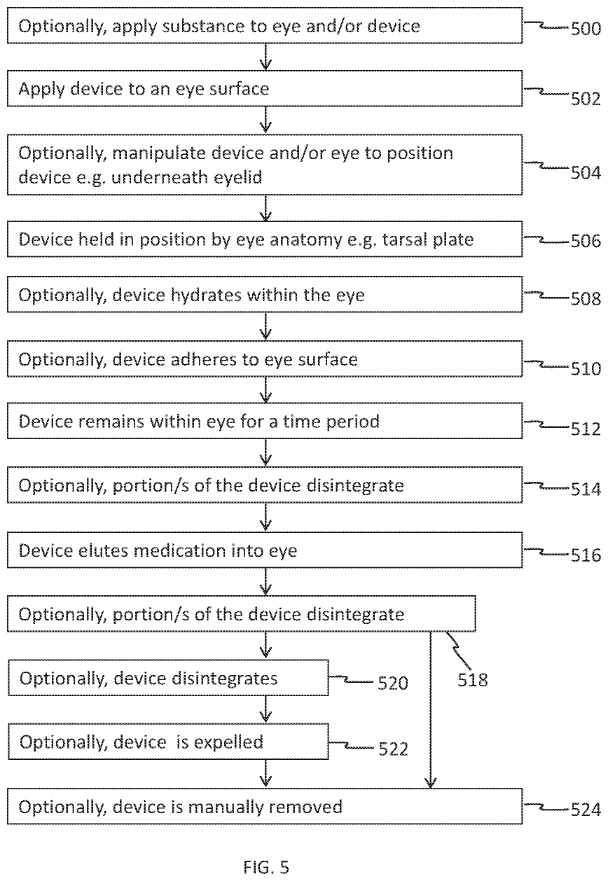

At 104, in some embodiments, the ophthalmic device exits the eye. Where, in some embodiments, the device exits the eye. Where, in some embodiments, exit is manual and/or the device is expelled by the eye. Where, in some embodiments, prior to exit of the ophthalmic device, the device disintegrates, at least partially. For example, where exit of the device from the eye, in some embodiments, includes one or more feature of one or more of steps 514-524 FIG. 5.

FIG. 2 is a method of treatment using an ophthalmic device, according to some embodiment of the invention.

At 200, in some embodiments, the ophthalmic device is positioned on the bulbar conjunctiva and at least partially underneath an eyelid of the eye e.g. fully underneath the eyelid. Where, in some embodiments, a retention portion of the ophthalmic device is positioned adjacent to an opening of the eyelid and deeper into the conjunctival sac than a tarsal plate of the eyelid e.g. when the eyelid is open and closed.

At 202, in some embodiments, interaction between the retention portion, the eyelid and/or eyeball, prevents escape of the ophthalmic device from underneath the eyelid. In some embodiments, interaction between the tarsal plate of the eyelid with the slope and/or height of the device acts to retain the device within the eyelid, behind the tarsal plate. In some embodiments, interaction between the tarsal plate and the device is associated with deformation of tissue of the eyelid around the device. In some embodiments, alternatively or additionally to force of the tarsal plate, reactive force of the tissue from deformation of the eyelid acts on the device to prevent movement of the device e.g. out of the eyelid and/or deeper into the cul-de-sac of the eye. Alternatively or additionally, in some embodiments, force between the eyeball and a posterior surface of the device acts to prevent movement of the device, where, in some embodiments, the force includes reactive force of pressure of the eyelid onto the device and then to the eyeball and/or suction forces between the device and eyeball.

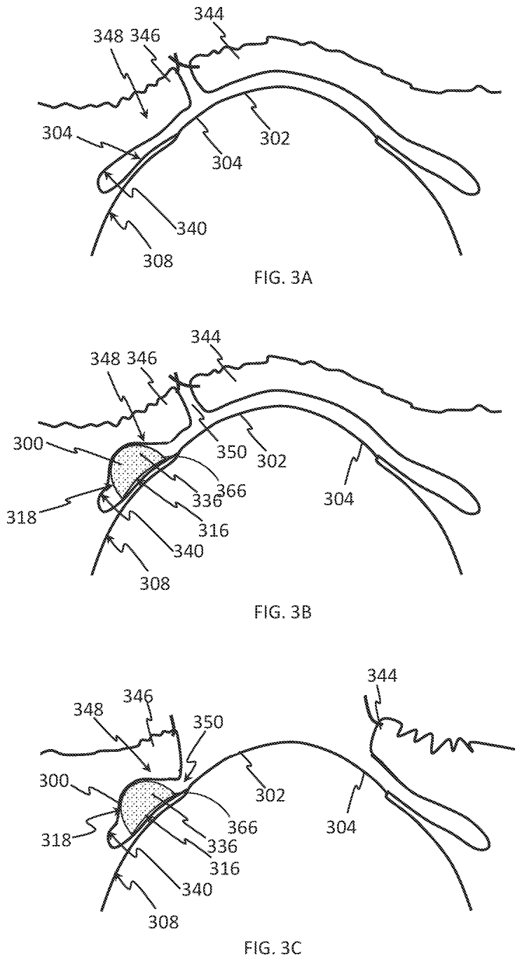

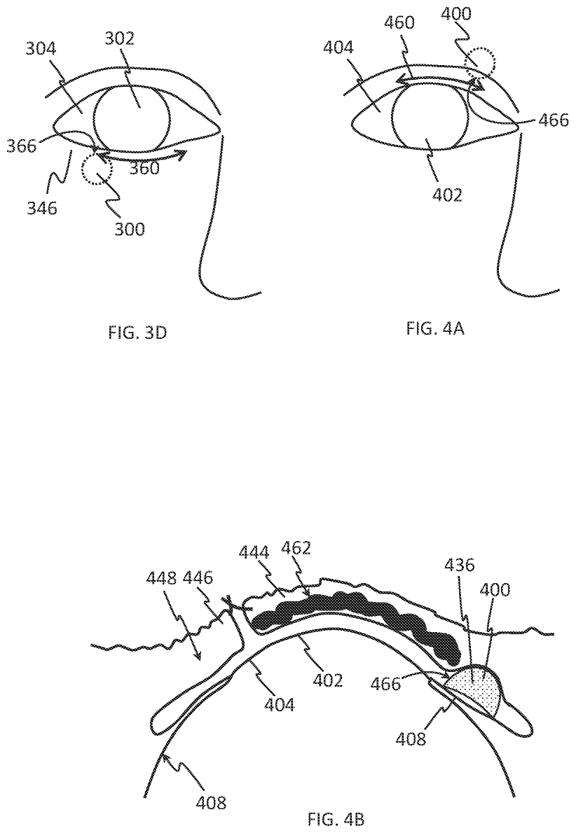

FIG. 3A is a simplified schematic cross sectional view of an eye.

FIG. 3B is a simplified schematic cross sectional view of an ophthalmic device 300 in position on an eye surface, when the eye is closed, according to some embodiments of the invention.

FIG. 3C is a simplified schematic cross sectional view of an ophthalmic device 300 in position on an eye surface, when the eye is open, according to some embodiments of the invention.

In some embodiments, cross sections of FIG. 3A-C are cross sections taken in an superior-inferior direction e.g. are sagittal cross sections of the eye.

In some embodiments, device 300 is covered by a lower eyelid 346 when the eye is closed (FIG. 3B) and when the eye is naturally open (FIG. 3C). In some embodiments, device 300 is positioned close enough to the eyelid opening e.g. on the bulbar conjunctiva and not deep into the cul-del-sac direction that manual movement, of the eyelid (e.g. without causing the user discomfort) is sufficient to reveal the device, at least partially.

In some embodiments, device 300 is positioned on an eye surface which is a portion of a surface of conjunctiva around an eyeball 308. In some embodiments, ophthalmic device 300 resides on a bulbar conjunctiva portion of a sclera, a region of conjunctiva adjacent to the cornea and in between the fornix conjunctiva 340 and cornea 302.

In some embodiments, device 300 has a body including a posterior surface 316, and an anterior surface 318. In some embodiments, device 300 has a retention portion 336 which is, in some embodiments, a portion of device 300 adjacent to an opening 350 of eyelid 346. In some embodiments, retention portion 336 is sufficiently thick at a short distance from a leading edge 366 of device 300 and/or presents a sufficiently steep slope to eyelid 346 that device 300 is retained underneath eyelid 346, for example, by a tarsal 348 plate. In some embodiments, the body of the ophthalmic device is not hollow. In some embodiments, the body of the ophthalmic device is made of a continuous material, meaning it does not comprise macroscopic gaps and/or empty areas within it. In some embodiments, a gap or empty area is defined as macroscopic if it is larger than 0.2 mm.

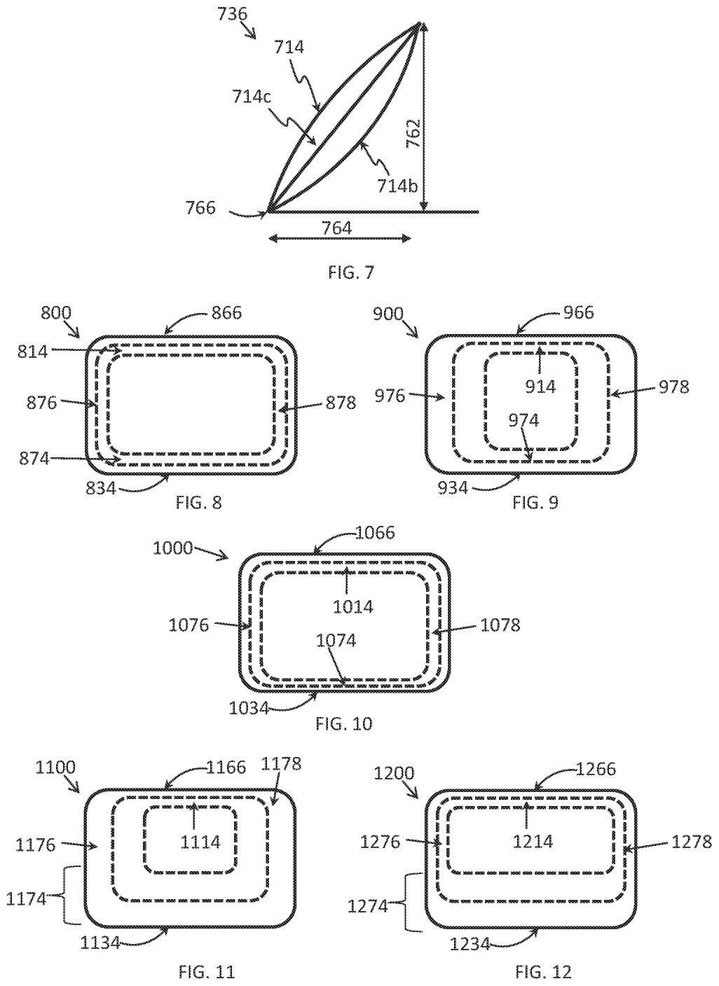

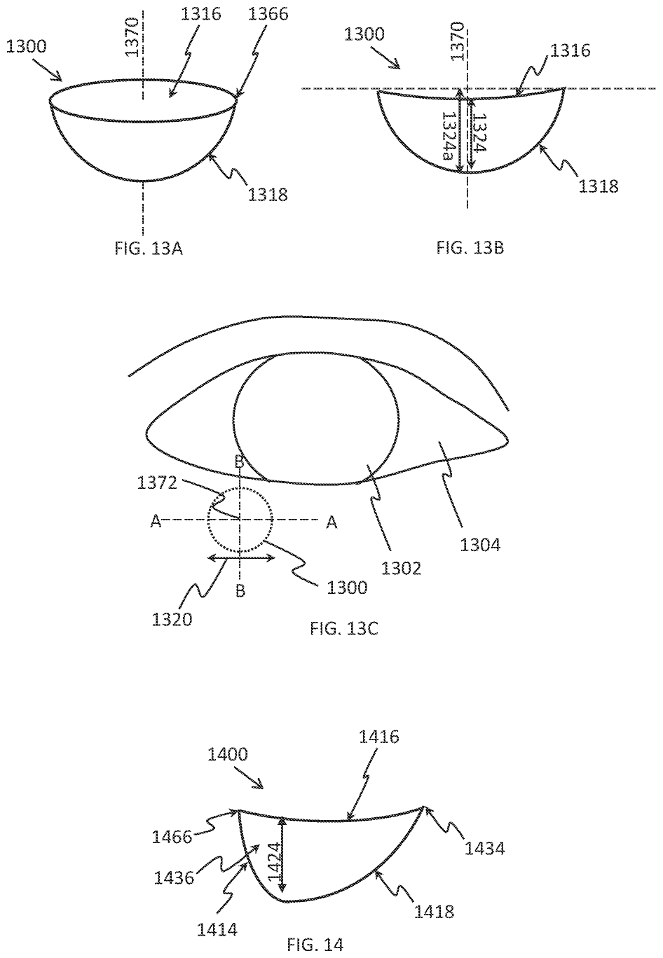

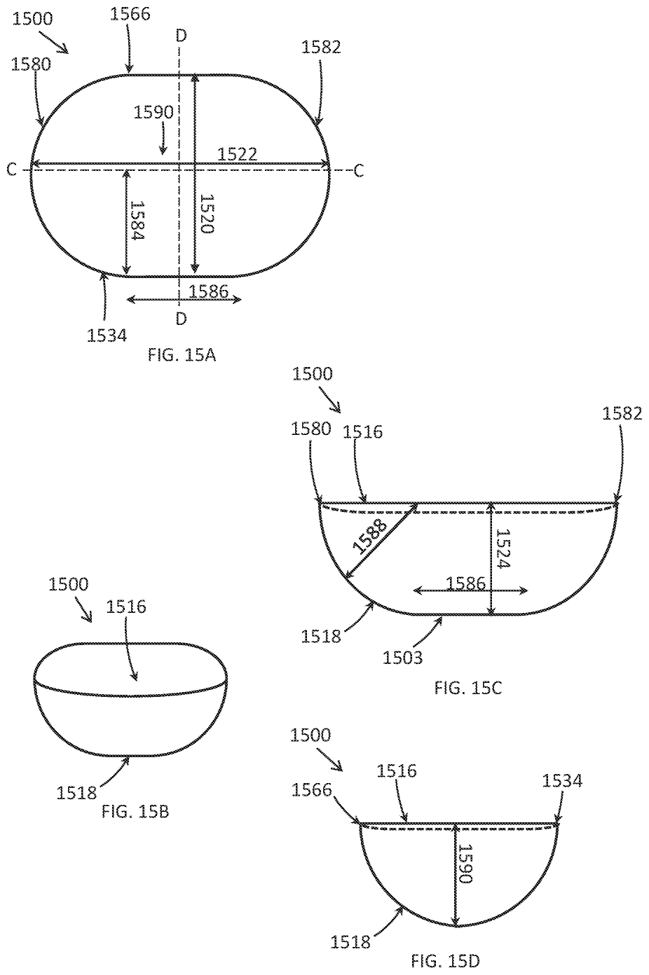

In some embodiments, retention portion 336 includes one or more feature of one or more of retention portions 636 FIGS. 6A-B, 736 FIG. 7, 1336 FIGS. 13A-C, 1436 FIG. 14, 1536 FIGS. 15A-D and/or other retention portion/s as described elsewhere in this document.

In some embodiments, reactive force associated with deformation of eyelid 346 and/or conjunctiva 308 around device 300 and/or interaction between tarsal plate 348 and retention portion 336 act to hold device 300 in position on the eye surface and/or underneath eyelid 346.

FIG. 3D is a simplified schematic top view of an ophthalmic device 300 in position on an eye surface, according to some embodiments of the invention.

FIG. 3D, in some embodiments, illustrates ophthalmic device 300 positioned on an eye surface (e.g. a sclera), for example, on an inferior portion of the bulbar conjunctiva underneath the lower eyelid 346 e.g. entirely underneath the lower eyelid 346.

FIG. 4A is a simplified schematic top view of an ophthalmic device 400 in position on an eye surface 404, according to some embodiments of the invention.

FIG. 4A, in some embodiments, illustrates ophthalmic device 400 is positioned an eye surface 404 (e.g. a sclera), for example, on a superior portion of the bulbar conjunctiva underneath the upper eyelid 444 e.g. entirely underneath the upper eyelid 444. In some embodiments, when device 400 is positioned on a superior portion of the eye, positioning is deeper within the fornix (e.g. in comparison to position where the device is positioned on an inferior portion of the eye e.g. underneath the lower eyelid).

FIG. 4B is a simplified schematic cross sectional view of an ophthalmic device 400 in position on an eye surface 404, when the eye is closed, according to some embodiments of the invention.

Where, in some embodiments, eye surface 404 is a superior portion of the sclera. Where, in some embodiments, device is held underneath upper eyelid 344, for example, when the eye is open and closed.

In some embodiments, a depth to which device 400 is inserted and/or a length of the device extending underneath the upper eyelid is deeper than that of a device for use under the inferior eyelid. In some embodiments, increasing likelihood of the device remaining underneath the superior eyelid where, generally, the eyelid and tarsal plate have larger extent.

In some embodiments, reactive force associated with deformation of eyelid 444 and/or conjunctiva 408 around device 400 and/or interaction between superior tarsal plate 462 and retention portion 436 act to hold device 400 in position on the eye surface and/or underneath upper eyelid 444.

Referring now to both FIG. 3D and FIG. 4A, arrows 360, 460, in some embodiments, illustrate ranges of exemplary positions for device/s 300, 400 e.g. with respect to visible portions of an eye.

Where, in some embodiments, ophthalmic device 300 is positioned underneath a central portion of lower eyelid 346. Where, in some embodiments, ophthalmic device 400 is positioned underneath a central portion of upper eyelid 444.

For example, a central 2-10 mm, or 2-7 mm, or lower or higher or intermediate ranges or values e.g. as measured along a contour of a junction between the eyelid and an eye surface (e.g. sclera 304, 404).

For example, at least 1 mm, or 2 mm, or 5 mm, from the inner and/or outer eye corners e.g. as measured along a contour of a junction between the eyelid and the eye surface (sclera 304, 404 and/or cornea 302, 402).

For example, a central 30-80%, or 40-70%, or lower or higher or intermediate ranges or percentages. Where, in some embodiments, the portion is measured as a length along a contour of the eyelid (e.g. at a junction between the eyelid and the eye surface) with respect to a total length measured along the contour of the eyelid between corners of the eye, when the eye is closed and/or open.

In some embodiments, device/s 300, 400 are symmetrical in shape, e.g. in one or more dimension. In some embodiments, device/s 300, 400 have a circular footprint e.g. when residing on an eye surface (e.g. sclera 304, 404).

### Exemplary Detailed Method

FIG. 5 is a method of use of an ophthalmic device, according to some embodiments of the invention.

At 500, in some embodiments, optionally, a substance is applied to an eye and/or to the ophthalmic device.

For example, in some embodiments, the device is stored in a dry or partially hydrated form and is hydrated before use (e.g. insertion into the eye). For example, by applying water and/or a hydrating solution to the device prior to positioning the device on an eye surface. In some embodiments, the device is soaked in a hydrating solution before applying to the eye e.g. for 5 seconds-20 mins. In some embodiments, the device is humidified by exposing to humid air and/or gas before applying to the eye e.g. for 1 minute-2 hours.

In some embodiments, one or more therapeutic material is applied to the device. In some embodiments, mucoadhesive material and/or lubricious material is applied to the device e.g. one or more portion of the device.

At 502, in some embodiments, the ophthalmic device is applied to an eye surface. In some embodiments, applying includes bringing the device into contact with the eye surface e.g. the sclera e.g. bulbar conjunctiva.

In some embodiments, applying includes retracting the eyelid under which the device is to be positioned and placing the device on an eye surface usually underneath the eyelid.

In some embodiments, the device is directly and/or manually applied to the eye surface. For example, by the patient themselves. In some embodiments, a caregiver applies the device. Optionally, in some embodiments, the device is applied using an applicator.

In some embodiments, for example, when the device is not rotationally symmetrical, the device is orientated prior to and/or during positioning on the eye surface. In some embodiments, an applicator is sized and/or shaped and/or has one or more indicator to aid positioning the device at a desired orientation within the eye. Orientations, for example, as described elsewhere in this document e.g. orientation with respect to a leading edge and/or shape (e.g. elongation) of the device.

At 504, optionally, in some embodiments, after application to the eye surface, the ophthalmic device is repositioned. For example, by manipulation of the eye and/or ophthalmic device and/or eyelid. For example, in some embodiments, the ophthalmic device is positioned protruding at least partially from underneath an eyelid and, in some embodiments, the ophthalmic device is repositioned (e.g. to be fully underneath the eyelid) by directly moving the ophthalmic device and/or by manipulating the eye (e.g. eyelid) to move the ophthalmic device. For example, in some embodiments, the ophthalmic device is placed on the sclera above the eyelid and then is manipulated in an inferior direction e.g. until it is covered by the eyelid and/or held in position by the tarsal plate. In some embodiments, suction forces between the device and the eye surface (e.g. the posterior surface of the device) are sufficiently low, at least initially, to enable repositioning of the device.

At 506, in some embodiments, the ophthalmic device is held in place by eye anatomy. For example, held underneath the eyelid e.g. by interaction between the tarsal plate and a retention portion of the device e.g. including one or more feature as illustrated in and/or described regarding device 300, tarsal plate 348 FIGS. 3B-C and/or device 400, tarsal plate 462 FIG. 4B.

At 508, in some embodiments, optionally, the device hydrates within the eye, for example, absorbing tear fluid.

At 510, optionally, in some embodiments, the ophthalmic device adheres to the eye surface.

Where, in some embodiments, adherence is associated with pressure between the ophthalmic device and the eye surface (e.g. posterior surface of the device). In some embodiments, curvature of the ophthalmic device posterior surface aids adherence of the device to the eye surface. In some embodiments, this adherence is amplified by pressure of the eyelid onto the device. In some embodiments, one or more sharp edge of the device aids adherence of the device to the eye surface. In some embodiments, one or more concavity and/or protrusion e.g. on a posterior surface of the device aids adhesion.

In some embodiments, when the device is applied to the eye surface (i.e. is in situ), material properties of a mucoadhesive layer of the device upon application and/or after the mucoadhesive layer properties change in situ (e.g. hydrate) adhere the device to the eye surface.

In an exemplary embodiment, adhesion of the ophthalmic device to the eye surface is sufficient to maintain the device on the eye surface (e.g. sclera) in event of escape of the device from underneath the eyelid. For example, for a short period of time for example, less than 10 minutes, for example, 1 minute-1 hour, for example, during blinking e.g. during a few blinks e.g. 1-10 blinks, or 1-100 blinks. In which time, in some embodiments, a user returns the device to underneath the eyelid e.g. according to one or more feature as described regarding step 504.

At 512, in some embodiments, the device remains on the eyeball for a time period. For example, remaining underneath the eyelid.

In some embodiments, the device is maintained in situ (in the eye) for a period of at least 1 minute, or for 1-10 minutes, or for at least 0.5 hours, or for 0.1-2 hours, or for 2-8 hours, or for over 8 hours, or for 8-24 hours, or 1-7 days, or 7 days-1 month, or 1-3 months, or 3 months-1 year, or 1-30 days, or 1 month-1 year, or lower or higher or intermediate durations or ranges.

In some embodiments, the device remains and/or is maintained in situ (residence time) and is expelled and/or removed from the eye after less than about 24 hours from administration. Where expelling and/or removal, in some embodiments, leaves the eye clean from the device (e.g. without leaving behind debris), for example, the eye being ready for an additional administration e.g. of an additional device.

At 514, optionally, in some embodiments, one or more portion of the device degrades and/or disintegrates.

In some embodiments, degradation of the device occurs by one or more of mechanical degradation (such as eyeball or eyelid movement), chemical degradation (such as tear fluid) or biological degradation (such as enzymatic activity).

In some embodiments, speed of degradation is increased by a user, for example, where, in some embodiments, pressure is applied to the device (e.g. manually) to break the device into more than one part and/or hasten such breakage. In some embodiments, upon breaking into more than one part, one or more of the parts are sized and/or shaped (e.g. having retention portion/s) to remain underneath the eyelid.

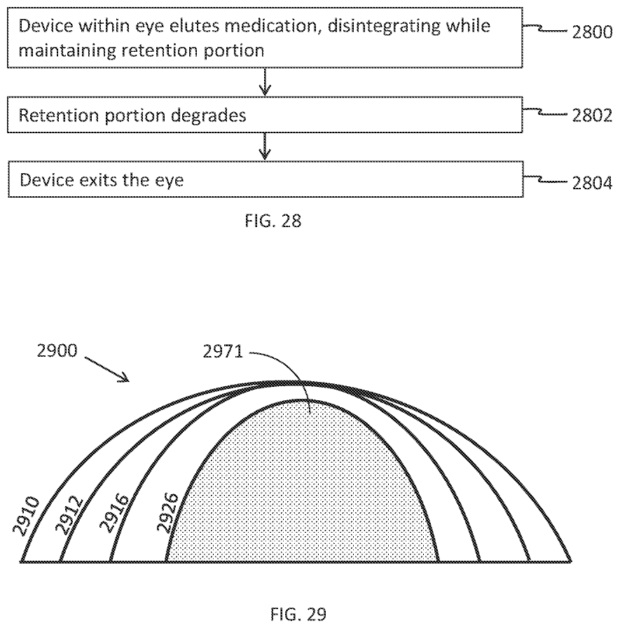

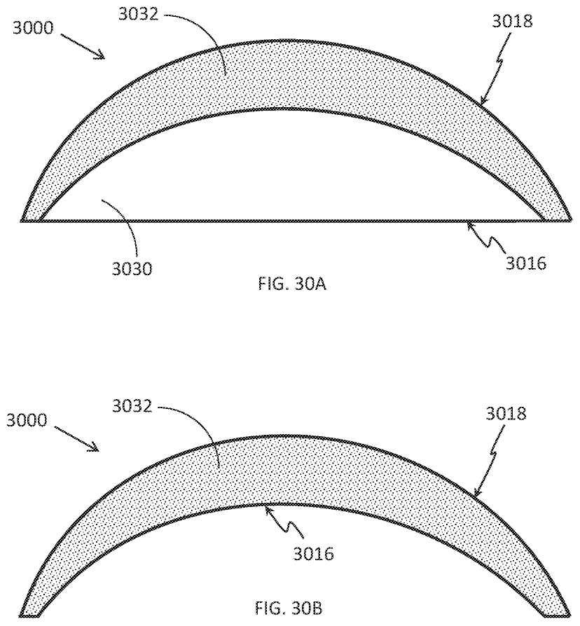

In some embodiments, the device includes portions which degrade at different rates. For example, including one or more feature as illustrated in and/or described regarding FIG. 28 and/or FIG. 29 and/or FIGS. 30A-B.

At 516, in some embodiments, the device performs treatment, for example, eluting medication into the eye. In some embodiments, elution is associated with degradation of eluting portion/s of the device.

At 518, optionally, in some embodiments, one or more portion of the device disintegrates. For example, a portion of the device disintegrating to reveal therapeutic material and/or to reveal additional therapeutic material. In some embodiments, portion/s of the device associated with device adhesion disintegrate. For example, a mucoadhesive layer. For example, portions which contribute to mechanical adhesion e.g. portion/s of a device edge and/or portion/s to change curvature of one or more surface of the device.

At 520, optionally, in some embodiments, the device disintegrates. For example, where disintegration of the device mechanically reduces adhesion of the device to the eye and/or reduces size of the device and/or breaks the device into pieces. In some embodiments, the entire device degrades after residence on the eye surface for a time period of between about 0.1 hours to 1 year, or less than 0.1 hours (e.g. immediately e.g. within a minute after placement on the eye).

At 522, in some embodiments, the device ophthalmic and/or portion/s of the ophthalmic device expelled from the eye naturally e.g. expelled by movement of the eyelid and/or eyeball and/or by blinking and/or tearing. In some embodiments, the device is expelled using manipulation of the eyelids. In some embodiments, portions of the device are expelled (e.g. when step 520 has occurred). In some embodiments, the device e.g. where the device remains in one portion, is expelled naturally. For example, upon degradation of adhesive related portion/s of the device.

At 524, optionally, in some embodiments, the ophthalmic device and/or portion/s of the device are removed e.g. manually. For example, by application of mechanical force e.g. pinching and/or applying suction and/or during use of a removal device (e.g. suction cup, tweezers) and/or by flushing of the eye.

In some embodiments, the ophthalmic device is removed by a combination of manual and natural processes. For example, in some embodiments, pressure is applied to the device to break it into more than one piece or to hasten such breakage. Where, in some embodiments, one or more of the pieces are then expelled naturally and/or dissolved.

In some embodiments, concurrent to one or more step and/or in between steps of the method described steps 500-524, one or more additional treatment method is administered concomitantly, sequentially or simultaneously. For example, in some embodiments, topical medication is applied e.g. during residence time of the device on the eye surface.

### Exemplary Ophthalmic Devices

FIG. 6A is a simplified schematic top view of an ophthalmic device 600 in position on an eye surface 604 underneath an eyelid 646, according to some embodiments of the invention.

In some embodiments, device 600 includes a retention portion 636 which is formed, when the device is in position on an eye surface, by a portion of device 600 adjacent to an opening of eyelid 646 (where, in some embodiments, eyelid 646 is a lower eyelid).

In some embodiments, retention portion 636 is a portion of device 600 extending from a leading edge 666 of the device inwards (e.g. towards a center of device 600).

In some embodiments, retention portion 636 extends inwards from leading edge 666 by at least a portion 630 of a depth 620 of device 600. In some embodiments, retention portion 636 extends laterally e.g. along leading edge 666 by at least a portion of a lateral length 622 of device 600.

FIG. 6B is a simplified schematic cross section of an ophthalmic device 600, according to some embodiments of the invention.

In some embodiments, FIG. 6B illustrates a cross section of device 600 of FIG. 6A where the cross section is taken along line BB of FIG. 6A. In some embodiments, FIG. 6B illustrates a range of cross sections of device 600 parallel to line BB of FIG. 6A e.g. for all of lateral extent 628 of retention portion 636. In some embodiments, FIG. 6B illustrates each of a range of cross sections of device 600 of FIG. 6A where the cross sections are along line BB of FIG. 6A or parallel to line BB of FIG. 6A.

In some embodiments, the body of the ophthalmic device 600 is symmetrical relative to its longitudinal axis (schematically shown by line AA) and relative to its transversal axis (schematically shown by line BB). For example, when looking at the ophthalmic device 600 (or other ophthalmic devices for example 300, 400, 800, 900, 1300, 1400, 1500, 1600, 1700, 1900, 2000, 2500, 2600, 2700, 2900, 3000, 3100) from the anterior surface, the device is symmetrical relative to its longitudinal axis (AA) and relative to its transversal axis (BB). In some embodiments device 600 has an anterior surface 618 and a posterior surface 616. In some embodiments, device 600 is a volume between the surfaces 616, 618, e.g. one or both of the surfaces curving to meet the other surface. For example, as illustrated in FIG. 6B where anterior surface 618 curves to meet posterior surface 616. In some embodiments, when resident on an eye surface, posterior surface 616 is adjacent to the eye surface (e.g. a sclera). In some embodiments, when resident on an eye surface, anterior surface 618 is adjacent conjunctiva of an eyelid (e.g. of a lower eyelid). In some embodiments, anterior surface 618 is convex and/or has a convex portion. In some embodiments, posterior surface 616 is convex and/or has a convex portion, as schematically shown by dotted line 616a in FIG. 6B.

In some embodiments, retention portion 636 has a holding height 662 at a holding distance 664 from leading edge 666. Where, in some embodiments, the holding height 662 is maintained (or exceeded) for a length 632 extending from leading edge towards trailing edge 634.

In some embodiments, retention portion 636 has concave and/or sufficiently steep slope 614 (e.g. as defined by slope of anterior surface 618) from leading edge 666 towards holding height 662. Where, in some embodiments, at a first distance 652 from leading edge 666, device 600 has a first height 654 and/or at a second distance 656 from leading edge 666, device 600 has a second height 658. In some embodiments, second distance 656 is at most 1 mm and first distance 652 is at least 1 mm.

Where distances from leading edge 666 are measured along anterior surface and/or from the leading edge along a surface as described regarding measurements of heights (e.g. elsewhere in this document).

In some embodiments, an angle θ of a slope of anterior surface 618 at leading edge 666 (e.g. of a tangent to the surface at the leading edge) or at an edge of the retention portion, is at least 35°, or at least 46°, or is 46-90°, or is 46-86°, or is 46-70°, or is lower or higher or intermediate angles or ranges. Where, in some embodiments, the angle is measured with respect to a planar surface on which the device is resting in a relaxed configuration. Where, in some embodiments, the angle is measured with respect to an eye surface, for example, an angle of the eye surface at the leading edge 666 and/or at an edge of the retention portion.

In some embodiments, anterior surface 618 has a radius of curvature 624 extending from leading edge 666 to holding height 662. Where, in some embodiments, radius of curvature 624 is equal to a peak height of the device e.g. as measured between posterior 616 and anterior 618 surfaces of device 600.

In some embodiments, e.g. as illustrated in FIG. 6B the device cross section (for one or more cross section) is symmetrical, e.g., has mirror symmetry. In some embodiments, the device is symmetrical in at least one direction, for example, about line BB and/or line AA as illustrated in FIG. 6A.

In some embodiments, ophthalmic device 600 is elongate. Where, in some embodiments, a length 622 of device 600 is larger than a width 620 of device 600. In some embodiments length 622 is 1.5-10 times, or lower or higher or intermediate multiples or ranges of width 620.

A potential advantage of an elongate device is that long sides 666, 634 of the device provide an increased surface for interaction with the eyelid/s e.g. for retention of the device within the eyelid and/or when removing the device using pinch removal.

In some embodiments, device 600 is positioned underneath eyelid 646 where a direction of elongation of device 600 is along a direction of an eyelid contour e.g. a contour of the opening to eyelid 646 and/or along a contour of a tarsal plate 648 of eyelid 646. A potential advantage of elongation of device 600, is an increased length of interaction of the device and the eyelid e.g. the tarsal plate (e.g. in comparison to the length of interaction of a device of the same volume and/or footprint size but less elongated). A potential advantage of elongation of the device, in some embodiments, is reduced likelihood of rotation of the device when in situ. In some embodiments, length 622 is larger than a depth of a cul-de-sac of the eye e.g. potentially preventing device 600 from rotating so that a direction of elongation is aligned in a direction extending from the eyelid opening to the bottom of the cul-de-sac.

In some embodiments, width 620 and/or length 622 of device 600 is 2-10 mm, or lower or higher or intermediate dimensions or ranges. For example, in some embodiments, length 622 is 5-10 mm and/or width 620 is 3-5 mm and/or height 662 and/or height 624 is 2-2.5 mm. In some embodiments, holding height 662 is at least 2 mm at a distance 664 from leading edge 666 of 2 mm. Alternatively or additionally, in so some embodiments, at distance 652 of 0.5 mm height 654 is at least 1.3 mm. Alternatively or additionally, in some embodiments, at distance 656 of 1 mm height 658 is at least 1.7 mm.

In some embodiments, where distance 664 is 2 mm, height 662 is larger than 0.5-5 mm, or 0.5-2 mm, or 1-2 mm, or lower or intermediate or higher heights or ranges.

In some embodiments, holding height 662 is maintained for a region of the cross section e.g. length 632 being at least 1 mm.

In some embodiments, peak height 624 is at most 5 mm, or 4 mm, or about 3 mm or about 2 mm or lower or higher or intermediate heights.

In some embodiments, for example, as described elsewhere in this document, anterior surface 616 is curved, e.g. concave. For example, including one or more feature of one or more of device 1300 FIG. 13A-C, device 1400 FIG. 14 and/or other device/s elsewhere in this document.

In some embodiments, device 600 is sufficiently rigid and/or resistance to forces (e.g. suction forces) when on an eye that the device does not deform significantly when in an eye so that, in some embodiments, heights as described above are heights above a one or more of:
a plane on which the relaxed device rests,
a plane connecting circumferential edges of the device or a majority (e.g. at least 80%, or at least 90%, or at least 95%, or at least 99%) of a circumference of the device,
a curved surface on which the relaxed device rests e.g. a surface with curvature in one or more direction of an eyeball, e.g. a sphere with radius of curvature of the eyeball.

In some embodiments, distances measured from leading edge 666 are measured along the plane used for measurement of height and/or along the posterior surface and/or along the anterior surface and/or a plane equidistant between the surfaces.

In some embodiments, heights e.g. as described regarding one or more of 654, 658, 662, 624 are heights of the device in situ above an eye surface. Where, in some embodiments, anterior surface 616 is curved (e.g. concave or convex). In some embodiments, forces (e.g. suction between the device and eyeball and/or pressure of the eyelid onto the device via the anterior surface) on the device act to flatten the device when the device is in situ on the eye surface.

Alternatively, the heights described are for a relaxed device, where, in some embodiments, flattening of the device reduces one or more height of the device by at most 30%, or at most 5-50%, or at most 5-30%, or at most 5-20%, or lower or higher or intermediate percentages or ranges from their original height/s. For example, height reducing by at most 30% corresponding to, in some embodiments, upon flattening the maximum height being at least 70% of an original (prior to flattening) maximum height.

FIG. 7 is a simplified schematic illustrating cross sections of portions of devices, according to some embodiments of the invention.

FIG. 7 illustrates, in some embodiments, an exemplary anterior surfaces 714, 714b, 714c of exemplary devices. Where, each of the device anterior surface contours, has a holding thickness 762 at a holding distance 764 from a leading edge 766 of the device. Convex anterior surface 714 (e.g. as described regarding anterior surface 618 of device 600 FIG. 6B) is illustrated for comparison purposes to, for example, an embodiment, where contour 714c extending from leading edge 766 to holding thickness 762 has a constant slope (e.g. within 10% of constant) and an embodiment where contour 714b is concave.

FIGS. 8-12 are simplified schematic top views of devices 800, 900, 1000, 1100, 1200, according to some embodiments of the invention.

In some embodiments, dashed lines are contour lines of an anterior surface of the device indicating, for the anterior surface, same distances above a height defined by a plane of footprints of the device as indicated in FIGS. 8-12 by solid lines.

Referring now to FIG. 8, in some embodiments, device 800 has the same slope and/or edge contour extending around a perimeter (or extending around at least 50%, or 70%, or 90%) of the device (e.g. as indicated by contour lines).

Where, for example, a slope 814 of the anterior surface adjacent to a leading edge 866 and a slope 874 adjacent to a trailing edge 834 are the same. In some embodiments, slopes 876, 878 on lateral sides of the device are the same e.g. the same as each other and/or the same as slope/s 814, 874. A potential benefit being that holding forces on the device (e.g. of the eyelid on the device) of the device are maintained (or changed less) upon rotation of the device (e.g. while the device is in position e.g. underneath an eyelid). In some embodiments, slopes 876, 878, of lateral sides of device 800 are different, for example, to configure device 800 to changing eye anatomy e.g. in a direction along a contour of an eyelid.

Referring now to FIG. 9 (and FIG. 11), in some embodiments, different edges of the device have different slopes. For example, in some embodiments, a slope 914, 1114 of a retention portion is located in a central region of device 900, 1100 leading edge 966, 1166 where, in some embodiments, one or both of lateral edges of the device have lower gradient slopes 976, 978, 1176, 1178.

Referring now to FIG. 10, in some embodiments, a slope 1074 of the anterior surface of the device extending to a trailing edge 1034 is higher than that of a retention portion slope 1014 adjacent to a leading edge 1066 of device 1000.

Referring now to FIG. 11 and FIG. 12, in some embodiments, a region of the device adjacent to a leading edge 1166, 1266 hosts the retention portion and a slope of the device 1174, 1274 extending towards a trailing edge has lower gradient slope. A potential benefit being increased comfort as the device tapers in thickness as it extends further underneath the eyelid towards the apex of the conjunctival fornix. In some embodiments, one or both lateral sides of the device also have lower gradient slope 1176, 1178 than retention portion slope 1114.

FIG. 13A is a simplified schematic view of an ophthalmic device 1300, according to some embodiments of the invention.

In some embodiments, FIG. 13A shows an isometric view of ophthalmic device 1300.

FIG. 13B is a simplified schematic cross sectional view of an ophthalmic device 1300, according to some embodiments of the invention.

FIG. 13C is a simplified schematic top view of an ophthalmic device 1300, according to some embodiments of the invention.

In some embodiments, ophthalmic device 1300 has an anterior surface 1316 and a posterior surface 1318. Where, in some embodiments, anterior surface 1318 curves to meet posterior surface 1316 at an edge 1366 of device 1300.

In some embodiments, ophthalmic device 1300 is symmetric, for example about one or more axis of symmetry. Referring to FIG. 13C, in some embodiments, ophthalmic device 1300 is symmetric about line AA and/or line BB.

In some embodiments, device 1300 has rotational symmetry, for example about a central axis 1370 which extends through a center 1372 of device top view. Where axis 1370 is perpendicular to one or both of anterior 1318 and posterior 1316 surfaces.

In some embodiments, device 1300 footprint (e.g. as illustrated in FIG. 13C) has a diameter of 2-10 mm, or 3-8 mm, or 4-7 mm, or 5-6 mm, or lower or higher or intermediate diameters or ranges. Where, in some embodiments, device 1300 has a height of 2-2.5 mm, or lower or higher or intermediate dimensions or ranges.

In some embodiments, posterior surface 1316 is curved, for example, in one or more direction. For example, in some embodiments, FIG. 13B illustrates a cross section taken of device along line AA and/or line BB of FIG. 13C.

A potential benefit of a symmetrical (e.g. rotationally symmetrical device) is that device characteristics e.g. regarding eye anatomy, are maintained if the device rotates in situ.

In some embodiments, not shown in FIG. 13A, 13B or 13C, anterior surface 1318, has higher radius of curvature (RA-Radius Anterior) than a radius of curvature of posterior surface 1316 (RP-Radius Posterior). For example, where, in some embodiments, RA is 1.2-10 or 1.2-5 times RP, or lower or higher or intermediate multiples or ranges.

In an exemplary embodiment, a radius of curvature of anterior surface 1318 is equal (or about equal, for example, within 20%, or within 10%, or within 5%, or lower or higher or intermediate percentages) of a maximum thickness 1324 of device 1300. For example device 1300 anterior surface 1318 having a hemisphere shape.

In an exemplary embodiment, a radius (e.g. half of width 1320) of the device footprint (FIG. 13C) and/or edges 1366 is 0.5-10 mm, or 1-10 mm, or 1-5 mm, or 2-4 mm, or 2.5-3 mm, or lower or higher or intermediate distances or ranges.

Hemisphere-shaped devices e.g. as described regarding FIGS. 13A-C, and having diameter 1320 of 5.5 mm, having a concave posterior surface 1316 and having height 1324a (as measured when the device is in a relaxed configuration on a planar surface) 2 mm were placed on the sclera under the lower eyelid of human volunteer's eyes and stayed there comfortably for several hours.

Elongated-Hemisphere-shaped devices e.g. as described regarding FIGS. 15A-D, having a concave posterior surface and having length 1522 of 6 mm, width 1520 of 4 mm, and height 1524 of 2 mm were placed on the sclera under the lower eyelid of human volunteer's eyes and stayed there comfortably for several hours.

FIG. 14 is a simplified schematic cross sectional view of an ophthalmic device 1400, according to some embodiments of the invention.

In some embodiments, curvature and/or slope of a surface 1418 of device 1400 changes along a cross section of the device. For example, in some embodiments, curvature of anterior surface 1418 changes moving from a leading edge 1466 towards a trailing edge 1434. Where leading edge 1466 and/or trailing edge 1434 include one or more feature as described regarding leading edge 666 and/or trailing edge 634 FIG. 6A, FIG. 6B respectively. For example, device 1400 having a retention portion 1436 with a slope 1414 and/or having a holding thickness 1424 where a curvature of anterior surface 1418 then reduces (e.g. from a maximal thickness portion of the device) towards trailing edge. Where, in some embodiments, slope 1414 and/or holding thickness 1424 have one or more feature of slope 614 and/or thickness 624 FIG. 6A and/or FIG. 6B. In some embodiments, such a cross section positions the retention portion non-centrally with respect to the top view and/or footprint of the device e.g. off center and towards a side of the device proximal to an opening of the eyelid when the device is in situ.

In some embodiments, (e.g. where anterior surface 1418 has changing curvature) posterior surface 1416 has constant curvature 1416. Alternatively, in some embodiments, posterior surface curvature 1416 also changes moving across one or more cross section of the device. For example, thinning of the device in a direction moving away from the leading edge, in some embodiments, being due to decrease in curvature of anterior surface combined with increase in curvature of the posterior surface. In some embodiments, (e.g. where anterior surface 1418 has constant curvature) thinning of the device in a direction moving away from the leading edge is due to increase in curvature of the posterior surface.

FIG. 15A is a simplified schematic top view of an ophthalmic device 1500, according to some embodiments of the invention.

FIG. 15B is a simplified schematic view of an ophthalmic device 1500, according to some embodiments of the invention.

In some embodiments, FIG. 15B shows an isometric view of ophthalmic device 1500.

FIG. 15C is a simplified schematic cross sectional view of an ophthalmic device 1500, according to some embodiments of the invention.

FIG. 15D is a simplified schematic cross sectional view of an ophthalmic device 1500, according to some embodiments of the invention.

In some embodiments, device 1500 is elongate. Where, in some embodiments, elongation of device 1500 includes one or more feature as described regarding device 600 FIG. 6A. In an exemplary embodiment, a device width 1520 is 1.1-10 times, or 1.1-5 times, or 1.2-3 times, or 1.2-2.5 times, or about 1.5-2 times a device length 1522, or lower or higher or intermediate multiples or ranges.

In some embodiments, a leading edge 1566 of device 1500 is straight and/or a trailing edge 1534 is straight. In some embodiments, leading edge 1566 and trailing edge 1534 are parallel (or within 1, or 3, or 5, or 10 degrees of parallel, or lower or higher or intermediate angles). In some embodiments, leading edge 1566 and/or trailing edge 1534 are straight where compared to, for example, lateral edges 1580 and 1582. In other words, leading edge 1566 and/or trailing edge 1534 may comprise straight portions, whereas lateral edges 1580 and 1582 are curved, and therefore do not comprise straight portions.

In some embodiments, one or both of lateral edges 1580 and 1582 are curved. A potential benefit being increased comfort for a user wearing the device. In some embodiments, one or both of edges 1580 has a radius of curvature 1584. In an exemplary embodiment, radius of curvature 1584 is half (or about half) of a device footprint width 1520.

In some embodiments, FIG. 15C illustrates a cross section of device 1500 taken laterally across the device e.g. along a direction of elongation e.g. along line CC (in FIG. 15A). In some embodiments, device cross section has a radius of curvature 1588 of an anterior surface 1518 extending from a first lateral edge 1580. In some embodiments, the cross section has the same radius of curvature 1588 for anterior surface 1518 extending from second lateral edge 1582 to a central region and/or a maximal thickness 1524 of the cross section. In some embodiments, a radius of curvature 1518 of the posterior surface, in one or more direction, is 1-15 mm, or 1-10 mm, or lower or higher or intermediate radii or ranges and/or has a ratio to that of the sclera being 0.1-2, or 0.5-1, or lower or higher or intermediate ratios or ranges.

In some embodiments, FIG. 15D illustrates a cross section of device 1500 taken in a direction of leading edge 1566 towards trailing edge 1534 e.g. along line DD (in FIG. 15A). In some embodiments, FIG. 15D illustrates cross sections of device 1500 extending along a central region 1590 of device 1500 e.g. as illustrated by arrow 1586. In some embodiments, the cross section of FIG. 15D includes one or more feature of the cross section of FIG. 6B. In some embodiments, a radius of curvature 1590 of anterior surface 1518 is the same as a maximal thickness 1590 of the cross section.

In some embodiments, one or more of radii of curvature 1584, 1588, 1590 are related. In some embodiments, one or more of radii of curvature 1584, 1588, 1590 with respect to one or more of each other, are the same, or are about the same, or are within 5%, or 10%, or 20%, or 30%, or lower or higher or intermediate percentages of each other.

In some embodiments, lateral sides of device 1500 have the same curvature towards lateral edges 1580, 1582, in directions extending from a central region 1590 of device 1500, in some embodiments, lateral edge portions of device anterior surface having a quarter hemisphere shape.

In some embodiments, FIG. 15C also illustrates (e.g. alternatively to FIG. 15D) a cross section of device 1500 taken along line DD (in FIG. 15A), the device, in some embodiments, having a planar central region of the anterior surface as defined by a base region 1502 of anterior surface 1518 in FIG. 15C.

In some embodiments, device 1500 has a maximal thickness, extending along a lateral central line CC of device 1500 footprint (FIG. 15A). In some embodiments, the maximal thickness extends along a central contour e.g. as illustrated by arrow 1586.

In an exemplary embodiment, width 1520 is 4.5 mm and length 1522 is 10 mm. Where, in some embodiments, thickness 1524 and/or one or more of radii of curvatures 1584, 1588, 1590 are about 2.25 mm.

In an exemplary embodiment, width 1520 is about 5 mm and length 1522 is about 6.5 mm. Where, in some embodiments, thickness 1524 and/or one or more of radii of curvatures 1584, 1588, 1590 are about 2.25 mm.

In an exemplary embodiment, width 1520 is about 4.5 mm and length 1522 is about 8.5 mm. Where, in some embodiments, thickness 1524 and/or one or more of radii of curvatures 1584, 1588, 1590 are about 2.25 mm.

In an exemplary embodiment, width 1520 is about 5.5 mm and length 1522 is about 10 mm. Where, in some embodiments, thickness 1524 and/or one or more of radii of curvatures 1584, 1588, 1590 are about 2.75 mm.

In an exemplary embodiment, width 1520 is about 4 mm and length 1522 is about 6 mm. Where, in some embodiments, thickness 1524 and/or one or more of radii of curvatures 1584, 1588, 1590 are about 2 mm.

In an exemplary embodiment, width 1520 is about 5.5 mm and length 1522 is about 8.5 mm. Where, in some embodiments, thickness 1524 and/or one or more of radii of curvatures 1584, 1588, 1590 are about 2.25 mm.

In an exemplary embodiment, width 1520 is about 4.5 mm and length 1522 is about 6.5 mm. Where, in some embodiments, thickness 1524 and/or one or more of radii of curvatures 1584, 1588, 1590 are about 2 mm. Where, in some embodiments, a weight of the device is 18 mg.

Referring now to both FIG. 15C and FIG. 15D, in some embodiments, posterior surface 1516 is curved, at least for a portion of the posterior surface 1516, in at least one direction. For example, as illustrated by dashed lines in FIG. 15C and FIG. 15D which illustrate alternative posterior surface/s. In some embodiments, the posterior surface of device 1500 is concave, in at least one direction.

A potential advantage of a concave posterior surface is increased adhesion of the device to an eye surface on which the device rests. In some embodiments, not shown in FIGS. 15A-D, portion/s of an anterior surface 1518, have higher radius of curvature RA than the posterior surface RP. For example, where, in some embodiments, RA is 1.2-10 times RP, or 1.2-5 times RP, or lower or higher or intermediate multiples or ranges.

Referring now to a cross section taken along line DD. In an exemplary embodiment, at 0.5 mm from leading edge 1566, height (and/or thickness) of the device is 1.32 mm, and at a distance of 1 mm from leading edge 1566, height (and/or thickness) of the device is 1.73 mm, and at a distance of 2 mm from leading edge 1566 height (and/or thickness) of the device is 2 mm. For example, in some embodiments, a radius of curvature 1588 and/or 1590 is 2 mm.

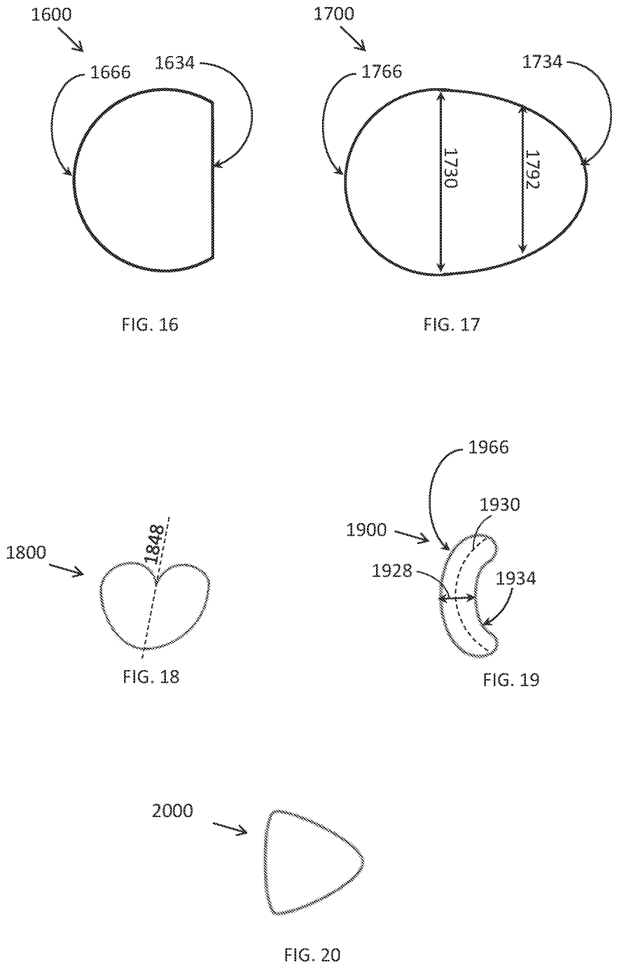

FIGS. 16-20 are simplified schematic top views of devices 1600, 1700, 1800, 1900, 2000, according to some embodiments of the invention.

Referring now to FIG. 16, in some embodiments, device 1600 is truncated at one or more side. In some embodiments, device 1600 has a curved edge 1666 and a low curvature edge 1634 (e.g. where, in some embodiments, low curvature edge 1634 is straight). In some embodiments, a region of a low curvature edge 1634 has a high curvature in a direction perpendicular to a plane of the footprint (and/or of FIG. 16). For example, in some embodiments, edge 1634 hosting a retention portion e.g. having feature/s as described regarding retention portions elsewhere in this document (e.g. retention portion 636 FIG. 6B).

Referring not to FIG. 17, in some embodiments, device 1700 has a top view shape which is tapered in one or more direction. For example, where, a maximum width 1730 of the device tapers towards one or more edge 1766, 1734 of device, for example, tapering more towards a first edge 1734 than towards a second edge 1766. In some embodiments, maximum width 1730 is located closer to one edge than the other e.g. towards the less tapered side of the device between maximum width 1730 and edge 1766. In some embodiments, second edge 1766 is a device leading edge and first edge 1734 is a trailing edge, leading and trailing edges including feature/s as described elsewhere in this document.

Referring now to FIG. 18, in some embodiments, an ophthalmic device 1800 has a shape having a narrow portion 1848 and/or indentation in the external form.

In some embodiments, device 1800 shape facilitates removal e.g. by pinching (e.g. manually and/or using a removal implement). Where, for example, in some embodiments, a portion configured to fold which is optionally in a central region (in one or more dimension) of the device (e.g. fold region; 1848 FIG. 18) is thinner and/or has a smaller footprint extent, e.g. facilitating folding after applying pinching force at one or both of larger extent regions on either side of the smaller footprint portion. In some embodiments, device 1800 includes two retention portions, for example, one at each side of fold region 1848.

Referring now to FIG. 19, in some embodiments, a device shape 1900 e.g. device footprint is selected for anatomical fit. For example, in some embodiments, device 1900 is shaped to position the device in contact with a portion of patient anatomy. For example, in some embodiments, an elongated device is curved and/or describes a curved shape. In some embodiments, ophthalmic device 1900 includes a curved shape top view and/or footprint. In some embodiments, a central longitudinal axis 1930 of the curved shape is 1-40 mm long, or 5-20 mm, long or lower or higher or intermediate ranges or lengths. In some embodiments, a radius of curvature of device 1900 is selected for the device to follow a curvature of an eyelid and/or portion of a cul-de-sac of an eyelid. Where, in some embodiments, a radius of curvature of 1928 is 20-100 mm, or 20-50 mm, or lower or higher or intermediate curvatures or ranges.

In some embodiments, the curve of device 1900 is sized and/or shaped to fit the eye e.g. a curve of an eyeball underneath an eyelid. In some embodiments, a retention portion is hosted adjacent to edge 1966. Alternatively or additionally, in some embodiments, a retention portion is hosted adjacent to edge 1934.

Referring now to FIG. 20, in some embodiments, device 2000 has one or more corners e.g. three corners for device 2000, for example, connected by sides. In some embodiments, corners of device 2000 facilitate removal of the device by pinching, for example providing the same potential distance (e.g. associated with extent) over which to apply pinch force (e.g. as a circular footprint device) but with reduced device footprint area. In some embodiments, a retention portion is hosted in a central region of device 2000 footprint. In some embodiments, retention portion/s are hosted adjacent to one or more edge of the multi-edged device.

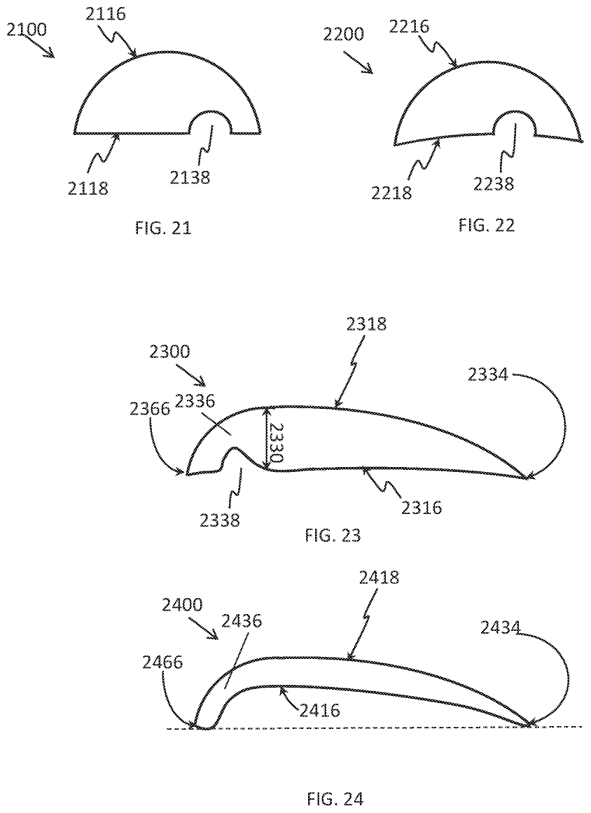

FIGS. 21-24 are simplified schematic cross sections of devices 2100, 2200, 2300, 2400 according to some embodiments of the invention.

Referring now to FIG. 21, in some embodiments, a posterior surface 2118 of device 1300 includes one or more concavity 2138.

Referring now to FIG. 22, in some embodiments, device 2200 includes both a curved posterior surface 2216 surface and one or more concavity 2238 on posterior surface 2218.

Referring now to FIG. 23, in some embodiments, device 2300 includes a concavity 2338 located on a posterior surface 2316. In some embodiments, concavity 2338 is located at a region of a retention portion 2336. For example, the concavity being adjacent to a leading edge 2366 of device, for example, within 1-5 mm of leading edge 2366. For example, located on a same portion (e.g. half) of the device as the retention portion 2336. Optionally, in some embodiments, curvature of an anterior surface 2318 reduces in a direction moving from a maximum thickness 2330 of device 2300 towards a trailing edge 2334 of the device.

Referring now to FIG. 24, in some embodiments, device 2400 has a highly concave posterior surface 2416, when compared for example, with the device 2300 shown in FIG. 23. In some embodiments, curvature of posterior surface 2416 changes along a length of the surface e.g. from first edge 2466 (which, in some embodiments is a leading edge) to second edge 2434 (which, in some embodiments, is a trailing edge). Posterior surface 2416, in some embodiments, having a higher curvature at a region adjacent to first edge 2466. In some embodiments, height (or thickness of the retention portion e.g. as defined within the overview section and/or with respect to FIGS. 6A-B) is measured between the dashed line illustrated in FIG. 24 and anterior surface 2418. Where, in some embodiments, the dashed line illustrates a planar surface.

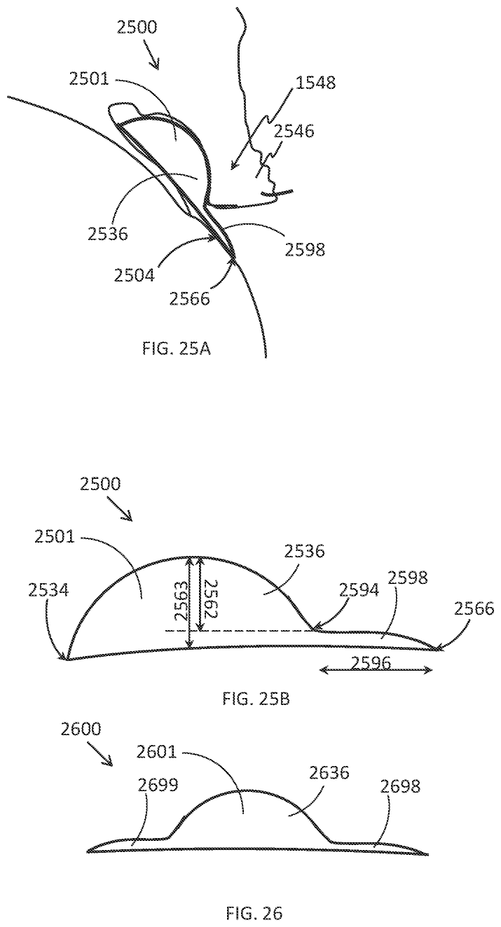

FIG. 25A is a simplified schematic cross sectional view of a device 2500 positioned on an eye surface, according to some embodiments of the invention.

FIG. 25B is a simplified schematic cross sectional view of a device 2500, according to some embodiments of the invention.

In some embodiments, FIG. 25A and FIG. 25B illustrate the same device and, in some embodiments, a same cross sectional view of the same device 2500.

In some embodiments, device 2500 includes an extending portion 2598 which is not covered by eyelid 2546 e.g. when the eye is open e.g. as illustrated in FIG. 25A. Where, in some embodiments, a body 2501 of device 2500 is located underneath eyelid 2546 (e.g. when the eye is open e.g. as illustrated in FIG. 25A).

In some embodiments, extending portion 2598 extends from body 2501 of device 2500 which includes a retention portion 2536.

In some embodiments, extending portion 2598 is thin and/or has a thin edge. In some embodiments, a step formed between a leading edge 2566 of device 2500 and the eye surface (or a planar surface contacting the device edges) is 1-500 microns, or 1-100 microns, or 10-50 microns, or lower or higher or intermediate ranges or steps. Thinness and/or the small step potentially presenting less surface area to interact with the eyelid as is moves over the area of the extending portion, e.g. during blinking and/or eyeball movement.

In some embodiments, extending portion 2598 has a length 2596 of 0.5-6 mm, or lower or higher or intermediate lengths or ranges. In some embodiments, body 2501 has one or more feature of devices e.g. as described elsewhere in this document. For example, one or more of the devices as described elsewhere in this document having an extending portion 2598 extending from the device leading edge.

In some embodiments, body 2501 of the device is held in position underneath the eyelid. For example, in some embodiments, body 2501 having a retention portion 2536 which includes one or more feature as described regarding and/or illustrated for retention portion 636 FIGS. 6A-B and/or elsewhere in this document.

In some embodiments, slope and/or thickness of retention portion 2536 is measured with respect to an edge 2594 of the retention portion with respect to extending portion 2598 and, for example, not by a leading edge 2566 of the device.

In some embodiments, extending portion 2598 is used to load a meniscus (the valley between the eyelid edge and the eyeball where the lacrimal/tear fluid accumulates and creates a reservoir of fluid) with one or more material and/or therapeutic agent.

FIG. 26 is a simplified schematic cross sectional view of a device 2600, according to some embodiments of the invention.

In some embodiments, a device 2600 has extending portions 2698, 2699 extending from a device body 2601 in more than one direction. In some embodiments, a single extending portion surrounds a device body 2601 footprint. Where device body 2601 includes one or more feature as illustrated in and/or described regarding device body 2501 FIGS. 25A-B. Where one or both of extending portions 2698, 2699 have one or more feature as illustrated in and/or described regarding extending portion 2598 FIGS. 25A-B.

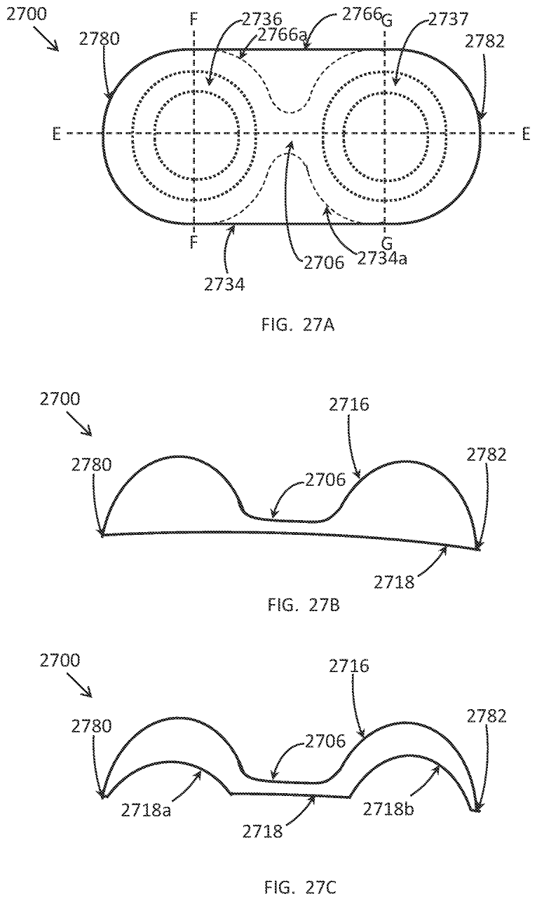

FIG. 27A is a simplified schematic top view of an ophthalmic device 2700, according to some embodiments of the invention.

FIG. 27B is a simplified schematic cross sectional view of an ophthalmic device 2700, according to some embodiments of the invention.

In some embodiments, FIG. 27B illustrates a cross sectional view of device 2700 of FIG. 27A, for example, taken along line EE of FIG. 27A.

In FIG. 27A, in some embodiments, dotted lines are contour lines of an anterior surface 2716 of device indicating, for the anterior surface, same distances above a height defined by a plane a device footprint as indicated in FIG. 27A by a solid line.

In some embodiments, device 2700 includes more than one retention portion 2736, 2737. Where, in some embodiments, retention portions 2736, 2737 are connected by a connecting portion 2706 which, in some embodiments, has a thickness which is below that required by a retention portion.

A potential advantage of multiple retention portions is potential retention of the device underneath an eyelid if a retention portion escapes from underneath the eyelid. A potential advantage of having more than one raised portion of the device (e.g. as provided by retention portions) is protruding surfaces easing pinch-removal e.g. as described elsewhere in this document. A potential advantage of having a connecting portion 2706, for example, instead of a single large retention portion is the ability to have a long device with reduced device volume.

Referring now back to FIG. 27A, which, in some embodiments, illustrates another embodiment having (instead of edge 2766), edge 2766a illustrated as a dashed line, which edge 2766a, in some embodiments, has an inlet, for example, extending towards central region 2706 where the central region 2706 has a smaller extent and/or footprint than central region 2706. In some embodiments, a device has a corresponding shape on an opposing edge 2734a illustrated as a dashed line (instead of edge 2734a).

In some embodiments, cross sections of device 2700 of FIG. 27A including edge inlet/s has the same cross sections as described for device 2700 of FIG. 27A without inlets.

FIG. 27C is a simplified schematic cross sectional view of a device 2700, according to some embodiments of the invention.

FIG. 27C illustrates an embodiment of one or both devices as illustrated by FIG. 27A, where, in some embodiments, each retention portion has a corresponding concave curve 2718a, 2718b, in posterior surface 2718a. Where concave surfaces include one or more feature as described regarding devices with a single retention portion e.g. elsewhere in this document. For example, including one or more feature of posterior surface 616 FIG. 6B and/or posterior surface 1516 FIGS. 15C-D and/or posterior surface 1316 FIG. 13B and/or FIG. 14. In some embodiments, the surfaces are concave in more than one direction, for example cross section taken along line FF and/or line GG having concave posterior surface, for example the cross section/s having one or more feature as described and/or illustrated regarding the cross section of FIG. 13B and/or FIG. 14.

### Exemplary Degradation

FIG. 28 is a process of ophthalmic device degradation, according to some embodiments of the invention.

At 2800, in some embodiments, a device positioned within an eye (e.g. underneath an eyelid of the eye degrades while maintaining structural feature/s of a retention portion. The retention portion acting to maintain the device underneath the eyelid. Where, during degrading, in some embodiments, the device elutes one or more therapeutic agent.

At 2802, in some embodiments, for example, after degradation and/or disintegration of other portion/s of the device, the retention portion degrades.

At 2804, in some embodiments, upon degradation of the retention portion, the device exits the eye, for example, upon exiting from underneath the eyelid.

FIG. 29 is a simplified schematic cross sectional view of a device 2900, according to some embodiments of the invention.

In some embodiments, contours 2910, 2912, 2916, 2926, illustrate an anterior surface of device 2900 over time, e.g. as device 2900 degrades during residence on an eye. In some embodiments, a slope and/or thickness of a retention portion is maintained e.g. through transition of device anterior surface from 2910 to 2912 and 2916 and only afterwards, in some embodiments do thickness (e.g. as illustrated) and/or slope of the retention portion reduce.

In some embodiments, device 2900 includes different portions with different degradation speeds. Where, in some embodiments, portion 2971 includes material which is less rapidly degraded (or is not degraded within the eye) than other portions of the device. In some embodiments, a less rapidly degraded portion of the device (e.g. portion 2971) is sized and/or shaped and/or positioned with respect to other portions of the device to maintain a retention portion e.g. during degradation of other portion/s of the device.

FIGS. 30A-B are simplified schematic cross sectional views of a device 3000, according to some embodiments of the invention.

In some embodiments, device 3000 includes different portions 3032, 3030 with different degradation times. In some embodiments, a posterior portion 3030 adjacent to a posterior surface 3016 of device 3000 is more rapidly degraded, than an anterior portion 3032 adjacent to an anterior surface 3018 of device 3000. For example as illustrated in transition in the device cross section illustrated in transition between FIG. 30A to FIG. 30B. In some embodiments, degradation of an anterior surface of device 3000, for example, combined, in some embodiments, with resistance of the device to collapse on the eye surface, maintains a slope and/or height of the device above the eye surface e.g. maintains a retention portion property/ies.

### Exemplary Ophthalmic Devices

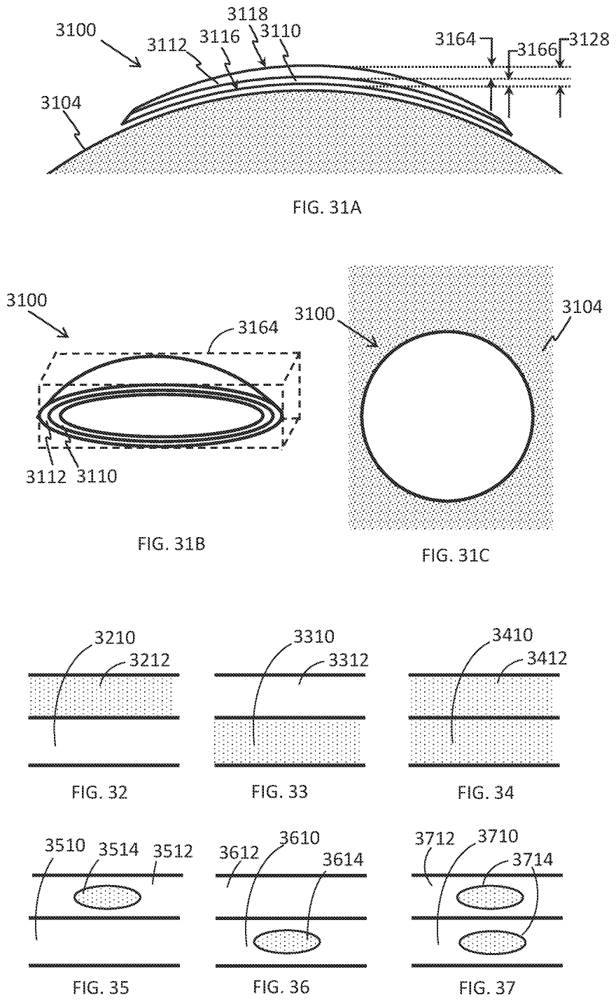

FIG. 31A is a simplified schematic cross section view of an ophthalmic device 3100 on an eye surface 3104, according to some embodiments of the invention.

FIG. 31B is a simplified schematic view of an ophthalmic device 3100, according to some embodiments of the invention.

FIG. 31C is a simplified schematic to view of an ophthalmic device 3100, according to some embodiments of the invention.

In some embodiments, device 3100 includes a posterior surface 3116 and an anterior surface 3118.

In some embodiments, device 3100 includes a plurality of layers 3110, 3112. Where, in some embodiments, layers generally follow surfaces of the device.

In some embodiments, a first layer 3110 is adjacent to and/or in contact with eye surface 3104 and/or forms at least a part of posterior surface 3116. In some embodiments, first layer 3110 includes mucoadhesive material.

In some embodiments, a second layer 3112 contacts, at least periodically, an inner surface of an eyelid, and/or forms at least part of anterior surface 3114. In some embodiments, second layer has a smooth outer surface and/or includes lubricious material. In some embodiments, one or both of first and second layers 3110, 3112, include and/or elute therapeutic material/s.

FIG. 31B, in some embodiments, illustrates determining of an extent of device 3100. Where, extent is, one or more dimension and/or average of two or more dimensions of a bounding box 3164 which is a smallest cuboid shape into which device 3100 fits.

In some embodiments, device 3100 includes one or more additional layer (e.g. as described elsewhere in this document.

In some embodiments, the ophthalmic device has a thickness 3128 (e.g. a maximum thickness and/or average thickness of a central 50% of the device) of less than 100 microns. In some embodiments, the ophthalmic device has a thickness of between about 1 to about 200 microns. In some embodiments, the device has a thickness of about 1, or about 2, or about 3, or about 4, or about 5, or about 6, or about 7, or about 8, or about 9, or about 10, or about 15, or about 20, or about 25, or about 30, or about 35, or about 40, or about 45, or about 50, or about 55, or about 60, or about 70, or about 80, or about 90, or about 100, or about 110, or about 120, or about 130, or about 140, or about 150, or about 160, or about 170, or about 180, or about 190, or about 200 microns. In some embodiments, the device has a thickness of between about 10 to about 100 microns.

In some embodiments, a first (e.g. mucoadhesive) layer 3166 and/or a second (e.g. lubricous) layer 3164 and/or an optional additional intermediate layer or layers (e.g. as described elsewhere in this document) has a thickness (e.g. a maximum thickness and/or average thickness at a central 50% of the device) of less than 100 microns. In further embodiments, one or more layer has a thickness of between about 1 to about 200 microns. In some embodiments, lubricious layer and/or a mucoadhesive layer and/or an intermediate layer has a thickness of about 1 microns, 2 microns, 3 microns, 4 microns, 5 microns, 6 microns, 7 microns, 8 microns, 9 microns, 10 microns, 15 microns, 20 microns, 25 microns, 30 microns, 35 microns, 40 microns, 45 microns, 50 microns, 55 microns, 60 microns, 70 microns, 80 microns, 90 microns, 100 microns, 110 microns, 120 microns, 130 microns, 140 microns, 150 microns, 160 microns, 170 microns, 180 microns, 190 microns, 200 microns. In some embodiments, one or more layer has a thickness of between about 1 to about 10 microns.

In some embodiments, one or more layer has about the same thickness as one or more other layer. In some embodiments, one or more layer has a different thickness than one or more other layer. In some embodiments, one or more layer has non-uniform thickness. For example, thinning from a central region of the device outward towards edge region/s of the device. For example, a layer having cavities and/or holes and/or protrusion/s.

FIGS. 32-37 are simplified schematic cross section views of portions of ophthalmic devices, according to some embodiments of the invention.

In some embodiments, in FIGS. 32-37, shaded areas indicate presence of therapeutic ingredient/s.

Referring now to FIG. 32 and FIG. 35, in some embodiments, a second layer 3212, 3512 (e.g. including one or more feature as described regarding second layer 3112 FIGS. 31A-C) comprises therapeutic ingredient/s. For example, dispersed within second layer 3112 and/or located within one or more region 3514 of second layer 3512.

Referring now to FIG. 33 and FIG. 36, in some embodiments, a first layer 3310, 3610 (e.g. including one or more feature as described regarding first layer 3110 FIGS. 31A-C) comprises therapeutic ingredient/s. For example, dispersed within second layer 3110 and/or located within one or more region 3614 of second layer 3610.

Referring now to FIG. 34 and FIG. 37, in some embodiments, both a first layer 3410, 3710 (e.g. including one or more feature as described regarding first layer 3110 FIGS. 31A-C) and a second layer 3412, 3712 (e.g. including one or more feature as described regarding second layer 3112 FIGS. 31A-C) comprises therapeutic ingredient/s. For example, dispersed within first layer 3410 and/or second layer 3412 and/or located within one or more region 3714 of second layer 3712 and/or first layer 3710. Where, in some embodiments, (not illustrated) one of the layers hosts a portion which has therapeutic material and the other layer has therapeutic material dispersed within it. In some embodiments, discrete region/s including therapeutic material include different therapeutic material than those layers which have therapeutic material dispersed within them. In some embodiments, discrete region/s are encapsulated by other material, the material for example, in some embodiments, eroding before the therapeutic material starts to elute into the eye.

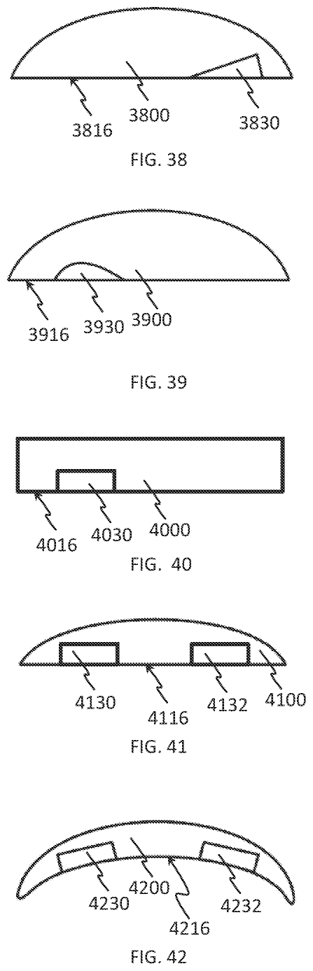

FIGS. 38-44 are simplified schematic cross sectional views of devices 3800, 3900, 4000, 4100, 4200, 4300, 4400, according to some embodiments of the invention.

In some embodiments, a device has a posterior surface which is flat or high radius of curvature e.g. posterior surface 3816 of device 3800 FIG. 38, e.g. posterior surface 3916 of device 39 FIG. 39, e.g. posterior surface 3406 of device 4000 FIG. 40, e.g. posterior surface 4116 of device 4100 FIG. 41. Where, in some embodiments, a high radius of curvature is more than 5 mm, or more than 10 mm, or more than 9.6 mm or 8-12 mm, or 9-11 mm, or lower or higher or intermediate radii of curvature or ranges.

In some embodiments, a device has a posterior surface which is concave, e.g. posterior surface 4216 of device 4200, e.g. posterior surface 4316 of device 4300, e.g. posterior surface 4416 of device 4400. Where, in some embodiments, concave posterior surface 4216, 4316, 4416, includes one or more feature as illustrated in and/or described regarding concave and/or curved posterior/s elsewhere in this document.

In some embodiments, a device and/or a cross section of a device includes a single cavity e.g. where, in some embodiments, element 3830, 3930, 4030 of FIG. 38-40 respectively is a cavity. Where, in some embodiments, the cavity 3830, 3930, 4030 is disposed on a posterior surface 3816, 3916, 4016, of the device.

In some embodiments, a device and/or a cross section of a device includes a single portion with different material, for example, mucoadhesive material e.g. where, in some embodiments, element 3830, 3930, 4030 of FIG. 38-40 respectively is a cavity. Where, in some embodiments, the mucoadhesive portion 3830, 3930, 4030 is disposed on a posterior surface 3816, 3916, 4016, respectively of the device.

Referring now to FIG. 41 and FIG. 42. In some embodiments, a device and/or a cross section of a device has a single portion with different material 4130, 4230 e.g. mucoadhesive material and has a single cavity 4132, 4232. Where, in some embodiments, cavity 4130, 4230 and different material portion 4132, 4232 are disposed on a posterior surface 4116, 4216 of device 4100, 4200 respective.

In some embodiments, a device and/or a cross section of a device has two portions of different material/s where portions 4130, 4132, of device 4100 and/or portions 4230, 4232 of device 4200 have the same material or different materials.

In some embodiments, a device 4100, 4200 and/or a cross section of the device has two cavities 4130, 4132, and 4230, 4232 respectively.

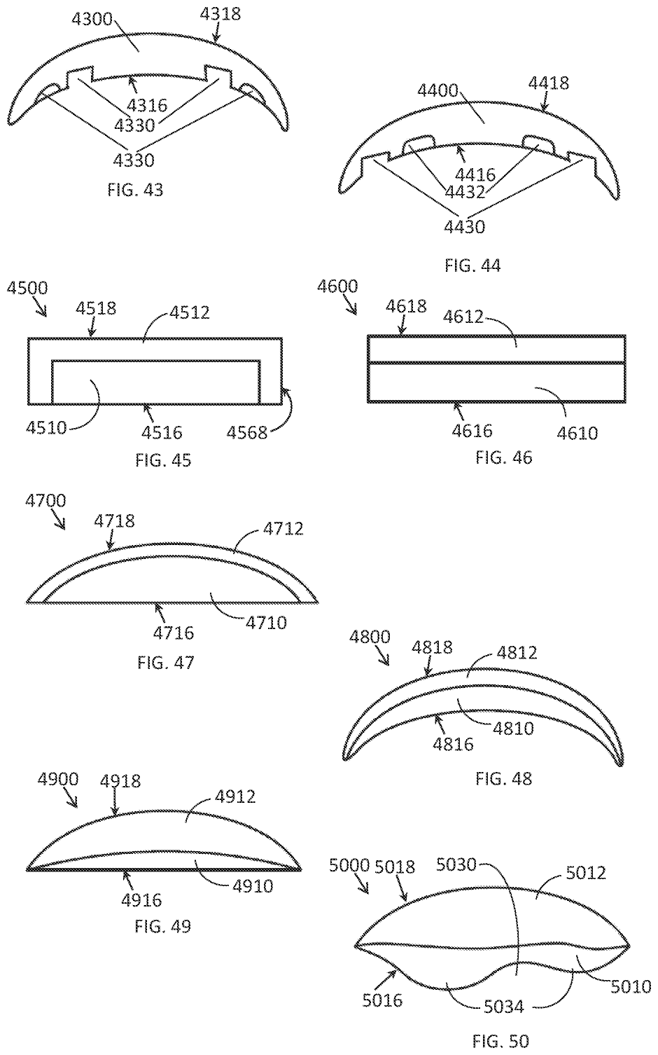

Referring now to FIG. 43 and FIG. 44, in some embodiments, a device 4300, 4400, has both more than one concavity 4330, 4432 and more than one different material portion 4332, 4432 all of which, in some embodiments, are disposed on posterior surfaces 4316, 4416 of device 4300, 4400 respectively. In some embodiments, one or more of the cavities and/or one or more of the different material portions are located within a body of the device and/or on a different device surface, for example, an anterior surface 4318, 4418 of the device.

Referring now to FIG. 43, in some embodiments, cavity/ies 4330 are disposed on posterior surface 4316 more centrally with respect to surface 4316 and/or device body 4300 than different material (e.g. mucoadhesive) portion/s 4332.

Referring now to FIG. 44, in some embodiments, different material (e.g. mucoadhesive) portion/s 4432 are disposed on posterior surface 4416 more centrally with respect to surface 4416 and/or device body 4400 than cavity/ies 4430.

FIGS. 45-50 are simplified schematic cross sectional views of double layer devices 4500, 4600, 4700, 4800, 4900, 5000, according to some embodiments of the invention.

In some embodiments, device 4500, 4600, 4700, 4800, 4900, 5000 has a posterior surface 4516, 4616, 4716, 4816, 4916, 5016 respectively and an anterior surface 4518, 4618, 4718, 4818, 4918, 5018 respectively.

In some embodiments, device 4500, 4600, 4700, 4800, 4900, 5000 includes a first layer 4510, 4610, 4710, 4810, 4910, 5010, and a second layer 4512, 4612, 4712, 4812, 4912, 5012. Where, in some embodiments, first layer has one or more feature as described regarding and/or illustrated regarding first layer 3110 FIGS. 31A-C and/or one or more of first layer 3210, 3210, 3310, 3410, 3510, 3610, 3710, FIGS. 31A-37 respectively. Where, in some embodiments, second layer has one or more feature as described regarding and/or illustrated regarding second layer 3112 FIGS. 31A-C and/or one or more of second layer 3212, 3212, 3312, 3412, 3512, 3612, 3712, FIGS. 31A-37 respectively.

In some embodiments, a device has a posterior surface which is flat or has low curvature (e.g. as defined elsewhere in this document) in dry and/or hydrated form. For example, posterior surface 4516 FIG. 45, posterior surface 4616 FIG. 46, posterior surface 4716 FIG. 47, posterior surface 4916 FIG. 49.

Referring now to FIG. 45 and FIG. 47, in some embodiments, a second layer 4512, 4712 (e.g. containing lubricous material) extends around a body of the device, for example, covering edge/s 4568 of the device and/or forming a part/s (e.g. at edge/s) of a proximal surface of the device 4516, 4716.

Referring now to FIGS. 45-47, in some embodiments, a first and/or second layer has about uniform thickness across an area of a device and/or along one or more cross section of the device. For example, layers 4510, 4512 FIG. 45, layers 4610, 4612, layer 4712 FIG. 47.

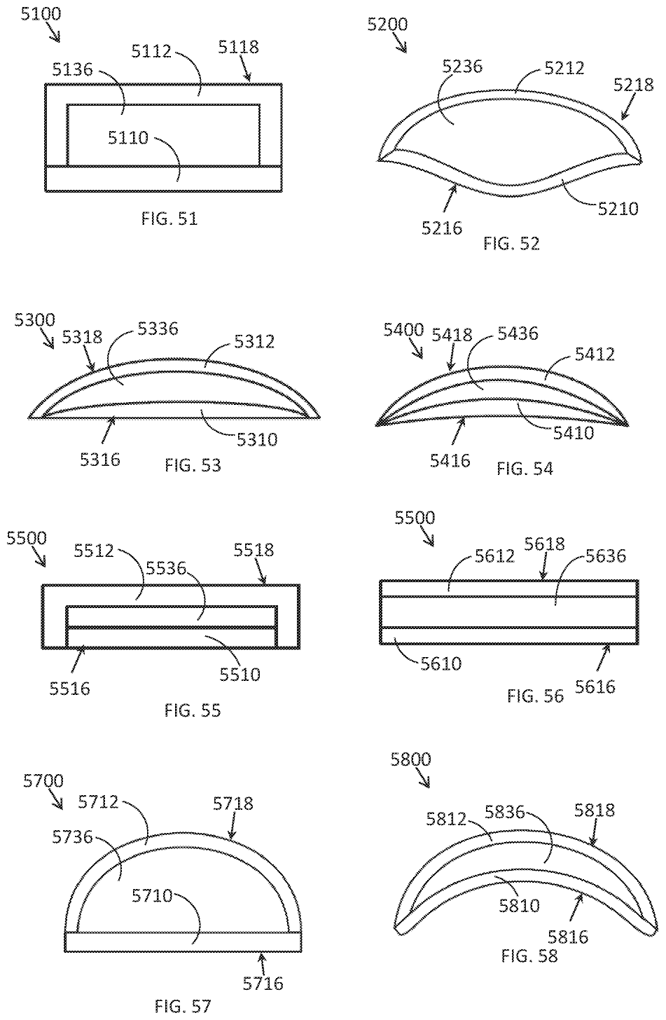

Referring now to FIGS. 47-50. In some embodiments, one or more layer has one or more of a maximal thickness at a center and/or central region of the device and reducing thickness moving towards edge/s of the device from the central region. For example, layer 4710 FIG. 47, layers 4810, 4812 FIG. 48, layers 4910, 4912 FIG. 49, layer 5012 FIG. 50.

FIG. 45, in some embodiments, illustrates a device 4500 with flat and/or low curvature posterior 4516 and anterior surfaces 4518, where second layer 4512 extends around edges of device 4500 to form a portion of anterior surface 4518. Where the layers are individually of about uniform thickness.

FIG. 46, in some embodiments, illustrates a device with flat and/or low curvature posterior 4616 and anterior surfaces 4618 and where the layers are individually of about uniform thickness.

FIG. 47, in some embodiments, illustrates a device 4700 with flat and/or lower curvature posterior surface 4716 and a convex anterior surface 4718 and where second layer 4712 extends around edges of device 4700 to form a portion of anterior surface 4718. In some embodiments, second layer 4712 is about uniform thickness. In some embodiments, first layer 4710 has a maximal thickness at a center and/or central region of the device and reducing thickness moving towards edge/s of device 4700 from the central region.

FIG. 48, in some embodiments, illustrates a device 4800 with both a concave posterior surface 4816 and a convex anterior surface 4818. Where, in some embodiments, both first layer 4810 and second layer 4812, have a maximal thickness at a center and/or central region of the device and reducing thickness moving towards edge/s of device 4800 from the central region.

FIG. 49, in some embodiments, illustrates a device 4900 where posterior surface 4916 is flat or has low curvature and where anterior surface 4918 is convex. Where, in some embodiments, both first layer 4910 and second layer 4912, have a maximal thickness at a center and/or central region of the device and reducing thickness moving towards edge/s of device 4900 from the central region.

FIG. 50, in some embodiments, illustrates a device 5000 where, in some embodiments, posterior surface has a bounding plane which is convex. In some embodiments, posterior has one or more protrusion 5034 and/or one or more cavity 5030. For example, a cross section with two protrusions 5034 defining between them a cavity 5030. In some embodiments, thickness of one or both layers is irregular. In some embodiments, posterior surface 5016 has an irregular shape, a convex shape, or a shape having multiple concavities.

FIGS. 51-58 are simplified schematic cross sectional views of multi-layer devices 5100, 5200, 5300, 5400, 5500, 5600, 5700, 5800 according to some embodiments of the invention.

In some embodiments, device 5100, 5200, 5300, 5400, 5500, 5600, 5700, 5800 has a posterior surface 5116, 5216, 5316, 5416, 5516, 5616, 5716, 5816 respectively and an anterior surface 5118, 5218, 5318, 5418, 5518, 5618, 5718, 5818 respectively.

In some embodiments, device 5100, 5200, 5300, 5400, 5500, 5600, 5700, 5800 includes a first (e.g. mucoadhesive) layer 5110, 5210, 5310, 5410, 5510, 5610, 5710, 5810, and a second (e.g. lubricous) layer 5112, 5212, 5312, 5412, 5512, 5612, 5712, 5812. Where, in some embodiments, the first layer has one or more feature as described regarding and/or illustrated regarding first layer 3110 FIGS. 31A-C and/or one or more of first layer 3210, 3210, 3310, 3410, 3510, 3610, 3710, FIGS. 31A-37 respectively. Where, in some embodiments, the second layer has one or more feature as described regarding and/or illustrated regarding second layer 3112 FIGS. 31A-C and/or one or more of second layer 3212, 3212, 3312, 3412, 3512, 3612, 3712, FIGS. 31A-37 respectively.

In some embodiments, device 5100, 5200, 5300, 5400, 5500, 5600, 5700, 5800 includes an intermediate layer 5136, 5236, 5336, 5436, 5536, 5636, 5736, 5836 respectively. In some embodiments, the intermediate layer is disposed between the first and second layers. Where, in some embodiments, the intermediate layer is covered by other layers e.g. as illustrated in FIGS. 51-55 and FIGS. 57-58. In some embodiments, the intermediate layer includes therapeutic material (e.g. dispersed within the layer and/or in portion/s of the layer).

FIG. 51, in some embodiments, illustrates a device 5100 having flat and/or low curvature posterior 5116 and anterior 5118 surfaces. Where, in some embodiments, second surface extends to cover edges of device 5100 e.g. to enclose sides of intermediate surface 5136. In some embodiments, one or more (e.g. all) of first 5110, second 5112 and third layers 5136 have about uniform thickness. In some embodiments, a thickness of intermediate layer 5136 is thicker than one or more of the other layers, for example, 1.5-10 times thicker or lower or higher or intermediate multiples or ranges.

FIG. 52, in some embodiments, illustrates a device 5200 where a posterior surface 5216 is convex.

In some embodiments, thicknesses of a first 5210 and second layer 5212 are about the same and, in some embodiments about uniform. In some embodiments, an intermediate layer 5236 has a thicker central region, reducing in thickness towards edges of device 5200. In some embodiments, an average and/or maximal thickness of intermediate layer 5236 is at 1.5-10 times thicker than thickness of one or both of the first 5210 and second 5212 layers.

In some embodiments, a convex posterior surface, reduces suction between the eyeball and the device, potentially increasing comfort of the device for a user and/or casing removal of the device. In some embodiments, a device includes shape of anterior 5216 and posterior surfaces 5218 of device 5200 of FIG. 52 but has a different layer structure and/or interior composition as described in the previous paragraph.

FIG. 53 in some embodiments, illustrates a device 5300 where each of a first layer 5310, an intermediate layer 5336, and a second layer 5312 have a thicker central region, the layers reducing in thickness towards edges of device 5300. In some embodiments, second layer 5312 is disposed around device 5300 to form part of posterior surface 5316. In some embodiments, posterior surface 5316 has higher radius of curvature than anterior surface 5318 (e.g. radius of curvatures including one or more feature as described elsewhere in this document e.g. in the overview section)

FIG. 54, in some embodiments, illustrates a device 5400 where each of a first layer 5410, an intermediate layer 5436, and a second layer 5412 have a thicker central region, the layers reducing in thickness towards edges of device 5400. In some embodiments, thicknesses of the layers are about the same at a central region of the device and/or throughout one or more cross section of the device.

In some embodiments, device 5500 of FIG. 55 has the same features as device 5100 of FIG. 51 except intermediate layer 5536 is, in some embodiments, about the same thickness as one or both of a first layer 5510 and a second layer 5512.

FIG. 56, in some embodiments, illustrates a device 5600 where each layer 5610, 5612, 5636 have constant thickness along the illustrated cross section. In some embodiments, intermediate layer 5636 includes therapeutic material. In some embodiments, intermediate layer 5636 is thicker than one or both of the other layers 5612, 5610. For example, a thickness of 1.5-10 times that of the mucoadhesive 5610 and/or lubricious 5612 layers. In some embodiments, intermediate layer 5636 includes a plurality of layers.

FIG. 57, in some embodiments, illustrates a device 5700 which has a convex anterior surface 5718, and a flat or low curvature posterior surface 5716. In some embodiments, mucoadhesive layer 5716 has uniform thickness across one or more cross section of the device. In some embodiments, lubricous layer 5712 has uniform thickness on its path around a circumference of intermediate layer 5736. In some embodiments, intermediate layer 5736 is thicker in a central region of one or more cross section of device 5700 and, in some embodiments, has reducing thickness towards edge/s of the device. In some embodiments, lubricous layer 5712 does not form part of posterior surface 5716.

FIG. 58, in some embodiments, illustrates a device 5800 which has a convex anterior surface 5818, and a concave posterior surface 5816. In some embodiments, mucoadhesive layer 5816 has uniform thickness across one or more cross section of the device. In some embodiments, lubricous layer 5812 has uniform thickness on its path around a circumference of intermediate layer 5836. In some embodiments, intermediate layer 5836 is thicker in a central region of one or more cross section of device 5800 and, in some embodiments, has reducing thickness towards edge/s of the device. In some embodiments, lubricous layer 5812 does not form part of posterior surface 5816.

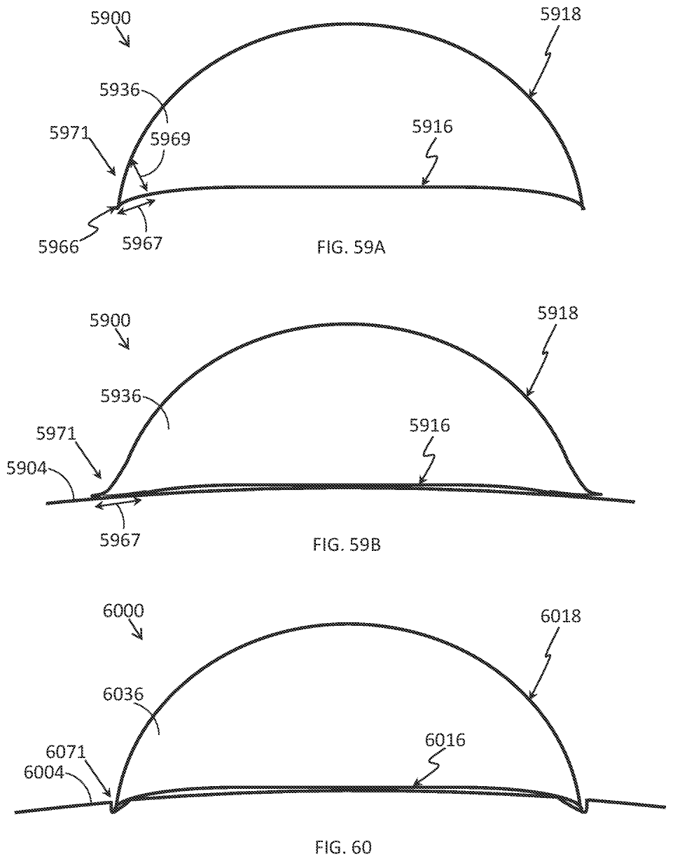

FIG. 59A is a simplified schematic cross section of an ophthalmic device 5900, according to some embodiments of the invention.

In some embodiments, device 5900 includes both a retention portion 5936 (e.g. including one or more feature as described regarding retention portions elsewhere in this document) and a thin edge 5971.

In some embodiments, an edge (e.g. circumferential edge) of the device has average thickness 5969, at distance 5967, as defined as a shortest distance between a posterior surface 5916 and an anterior surface 5918 of the device, of about 15 microns, or about 20 microns, or about 30 microns or about 50 microns. In some embodiments, the ophthalmic device has a thin edge of about 15 microns, or about 20 microns. In some embodiments, the ophthalmic device has an edge thickness of about 5-200 microns, or 5-200 microns, or lower or higher or intermediate thicknesses or ranges.

In some embodiments, distance 5967 is 0.1-1 mm, or 0.1-0.5 mm of the device, or lower or higher or intermediate distances or ranges. In some embodiments, distance 5967 is about 0.1 mm, or about 0.5 mm.

In some embodiments, thin edge 5971 extends for a region of device 5900. For example, a portion of a circumferential edge of the device. For example, for 20-99%, or 80-90%, or lower or higher or intermediate ranges or percentages, of a circumference of the device. For example, for a side of a device e.g. for the trailing edge only or the leading edge only. For example, for more than one side of the device e.g. leading edge and trailing edge, e.g. two opposing sides.

In some embodiments, FIG. 59A illustrates device 5900 in a relaxed state.

FIG. 59B is a simplified schematic cross section of an ophthalmic device 5900, according to some embodiments of the invention.

In some embodiments, FIG. 59B illustrates the device of FIG. 59A when in position of an eye surface (e.g. underneath an eyelid) In some embodiments, thin edge 5971 is flexible and conforms to an eye surface 5904, when device 5900 is on the eye surface.

FIG. 60 is a simplified schematic cross section of an ophthalmic device, according to some embodiments of the invention.

In some embodiments, FIG. 60 illustrates the device of FIG. 59A when in position on an eye surface, where the thin edge recesses into the eye surface, for example, as described in the overview section of this document.

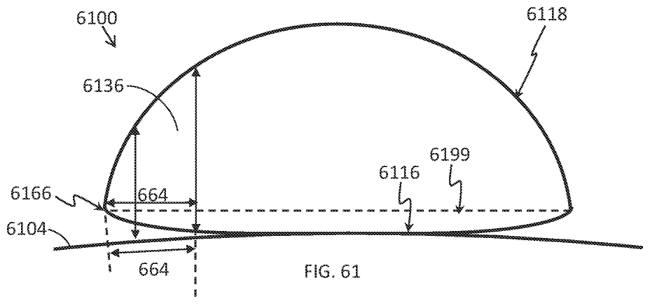

FIG. 61 is a simplified schematic cross section of an ophthalmic device 6100, according to some embodiments of the invention.

In some embodiments, device 6100 has a convex posterior surface 6116. In some embodiments, device 6100 has a retention portion 6136 which includes one or more feature of retention portions as described elsewhere in this document (e.g. retention portion 636 FIG. 6B) where, in some embodiments, heights are measured from an eye surface 6104 e.g. as illustrated by vertical arrows in in FIG. 61.

Alternatively, in some embodiments, heights are measured from a planar surface on which the device rests. Alternatively, in some embodiments, heights are measured from a plane 6199 connecting edges (or a majority of a circumferential edge) of the device or from a plane which delineates a largest area cross section of the device, the plane being located between anterior 6118 and posterior surfaces 6116.

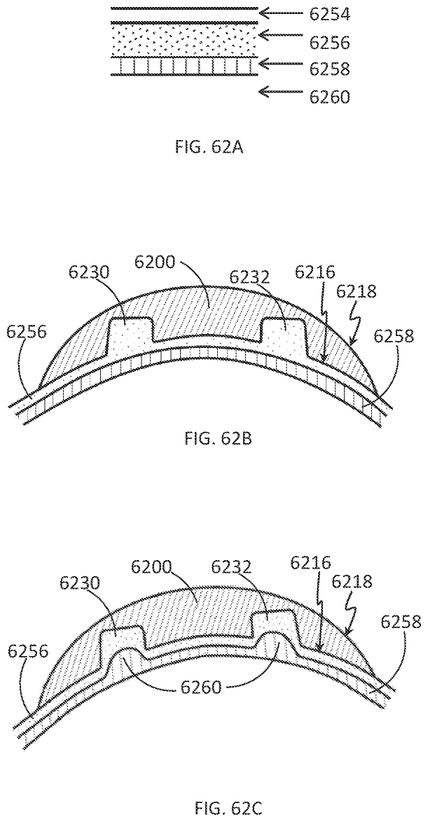

FIG. 62A is a simplified schematic cross sectional view of a portion of an eye surface, according to some embodiments of the invention.

FIG. 62B is a simplified schematic cross section view of an ophthalmic device 6200 on an eye surface, according to some embodiments of the invention.

FIG. 62C is a simplified schematic cross section view of an ophthalmic device 6200 on an eye surface, according to some embodiments of the invention.

In some embodiments, above a stroma 6260 is an eye epithelial layer 6258 over which resides an aqueous tear film layer 6256, above which resides a lipid layer 6254.

In some embodiments, FIG. 62A illustrates a cross section of eye anatomy, for example prior to application of device 6200 on the eye.

In some embodiments, FIG. 62B illustrates device 6200 and eye anatomy immediately and/or soon after application of device 6200.

In some embodiments, FIG. 62C illustrates device 6200 and eye anatomy a time period after application of device where, in some embodiments, the time period is about 1 minute to 1 hour after application.

Referring now to FIG. 62B. In some embodiments, device body 6200 includes a posterior surface 6216 and an anterior surface 6218. In some embodiments, body 6200 tapers in thickness towards edges of the body.

In some embodiments, device 6200 includes cavities 6230, 6232 which, in some embodiments, are disposed on posterior surface 6216.

In some embodiments, after application of the device tear fluid fills cavities 6232, 6234 epithelial 6258 as before application of the device. In some embodiments, tear fluid e.g. an aqueous and/or a lipid covers the device (not illustrated).

Referring now to FIG. 62C, in some embodiments, after a time period, in eye tissue conforms to a shape of device 6200. For example, epithelial tissue 6258 locally protrudes 6260 towards cavities 6230/6232 e.g. under suction at the cavity entrances. Potentially epithelial protrusion/s 6260 anchor device 6200 in position e.g. reducing likelihood of movement and/or expelling of the device.

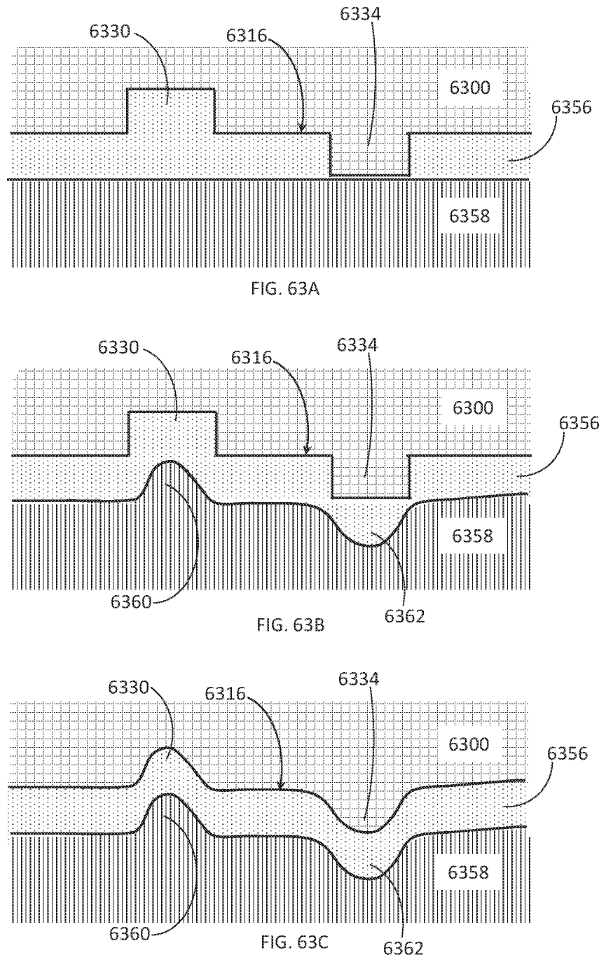

FIGS. 63A-C are simplified schematic cross sectional views of a portion of a device 6300 on an eye surface 6358, according to some embodiments of the invention.

In some embodiments, FIG. 63A illustrates device 6300 and eye anatomy 6358 immediately and/or soon after application of device 6300.

In some embodiments, FIG. 63B illustrates device 6300 and eye anatomy 6358 a first time period after application of device. Where, in some embodiments, a first time period is 1 minute to 1 hour.

In some embodiments, FIG. 63C illustrates device 6300 and eye anatomy 6358 a second time period after the cross section illustrated in FIG. 63B, for example, a further 1 minute to 1 hour after illustration of device 6300 and eye anatomy 6358 in FIG. 63B.

Referring now to FIG. 63A. In some embodiments, device body 6300 includes a posterior surface 6316 which includes a cavity 6330 and a protrusion 3034.

In some embodiments, after application of the device tear fluid fills cavities 6332, 6334 and epithelial 6358 is as before application of the device.

Referring now to FIG. 63B, in some embodiments, after a first time period, in eye tissue conforms to a shape of device 6300. For example, epithelial tissue 2058 locally protrudes 6360 towards cavity 6330 and/or recesses to form a recession 6362 in pliable epithelia tissue at a region adjacent to protrusion 6334. Where recessed material, in come embodiments, potentially anchors device 6300, potentially reducing likelihood of movement and/or expelling of device 6300.

Referring now to FIG. 63C, in some embodiments, biodegrading and/or bioerosion and/or has mechanically erosion of device 6300 has occurred. Where, in some embodiments, protrusion 6334 has reduced in size (e.g. in one or more dimension) and/or corner/s of protrusion 6334 have rounded. Where, in some embodiments, cavity 6330 corners have been rounded. In some embodiments, (not illustrated) the cavity becomes broader in one or more dimension and/or changes depth e.g. becomes shallower or deeper.

In some embodiments, proximal surface 6316 of FIG. 63C illustrates exemplary proximal surface shape, for example, including protrusion/s 6334 and/or cavity/ies 6330.

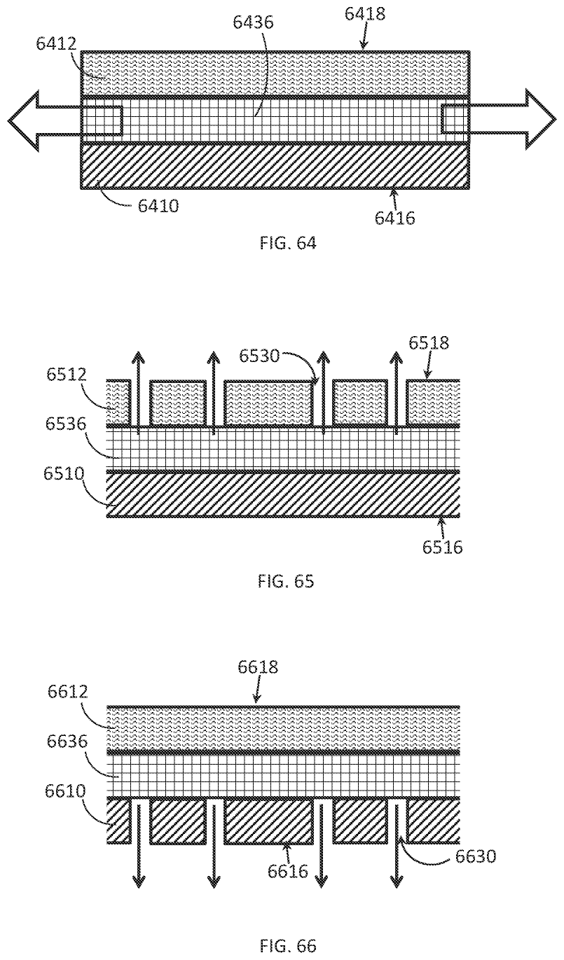

FIGS. 64-66 are simplified schematic cross sectional views of multi-layer devices 6400, 6500, 6600, according to some embodiments of the invention.

FIGS. 64-66, in some embodiments, illustrate therapeutic ingredient elution as illustrated in the figures by arrows.

Referring now to FIG. 64, in some embodiments, an intermediate layer 6436 includes therapeutic material and is not covered by other layer/s at edges of device 6400. The layer, in some embodiments, eluting therapeutic ingredient/s through edges e.g. as illustrated by arrows.

Referring now to FIG. 65, in some embodiments, (e.g. alternatively or additionally to eluting through edges of the device) an intermediate layer 6536 includes therapeutic material and is not fully covered by a second lubricous layer 6512, therapeutic ingredient/s eluting through the second layer. For example, through channel/s and/or holes 6530 in the layer 6512 e.g. as illustrated by arrows on FIG. 65. In some embodiments, alternatively or additionally to having channel/s and/or holes, second layer 6512 includes material which allows diffusion through the material of therapeutic ingredient/s.

Referring now to FIG. 66, in some embodiments, (e.g. alternatively or additionally to eluting through edges and/or through a second lubricious surface 6612 of the device) an intermediate layer 6636 includes therapeutic material and is not fully covered by a first mucoadhesive layer 6510, therapeutic ingredient/s eluting through the first layer. For example, through channel/s and/or holes 6630 in the layer 6610 e.g. as illustrated by arrows on FIG. 66. In some embodiments, alternatively or additionally to having channel/s and/or holes, first layer 6610 includes material which allows diffusion through the material of therapeutic ingredient/s

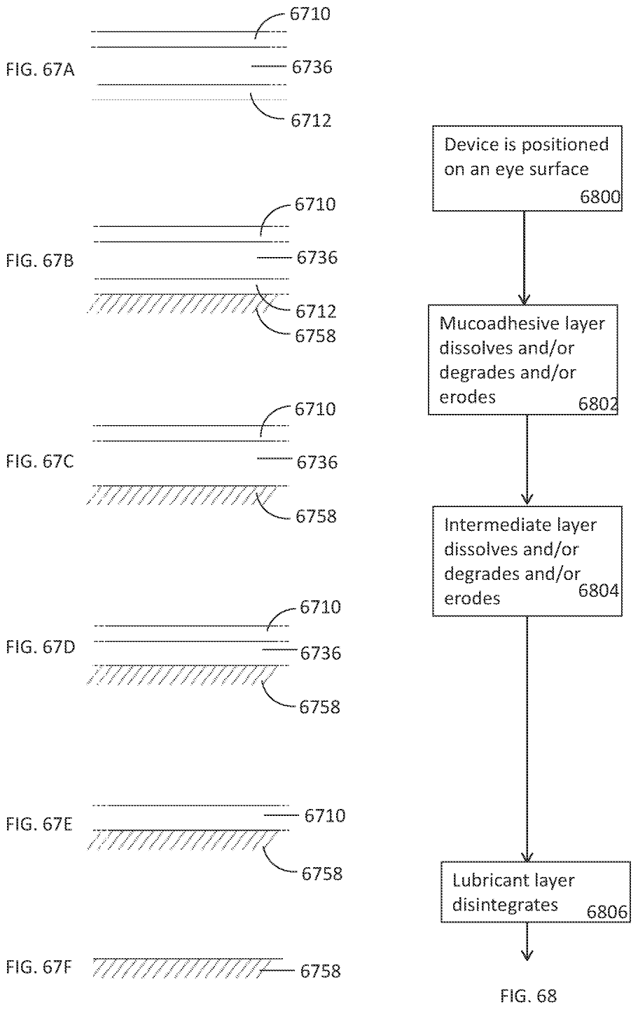

FIGS. 67A-F are simplified schematic cross sections of a portion of a device as residence time of the device progresses, according to some embodiments of the invention.

FIG. 68 is a flowchart of a disintegration progression of a device, according to some embodiments of the invention.

FIG. 67A, in some embodiments, illustrates the device layer structure before the device is placed on an eye surface. The device including a first mucoadhesive layer 6710, an intermediate layer 6736 and a second lubricous layer 6712.

At 6800, in some embodiments, device 6700 is positioned on an eye surface 6758.

At 6802, in some embodiments, mucoadhesive layer 6710 degrades and/or dissolves and/or erodes, for example, transitioning device 6700 from the configuration illustrated in FIG. 67B to that illustrated in FIG. 67C.

At 6804, in some embodiments, intermediate layer 6736 partially degrades and/or dissolves and/or erodes, for example, transitioning device 6700 from the configuration illustrated in FIG. 67C to that illustrated in FIG. 67D and then to that illustrated in FIG. 67E.

At 6806, in some embodiments, lubricious layer 6712 dissolves, and/or degrades and/or erodes, for example, transitioning the eye from the configuration illustrated in FIG. 67E to that illustrated in FIG. 67F.

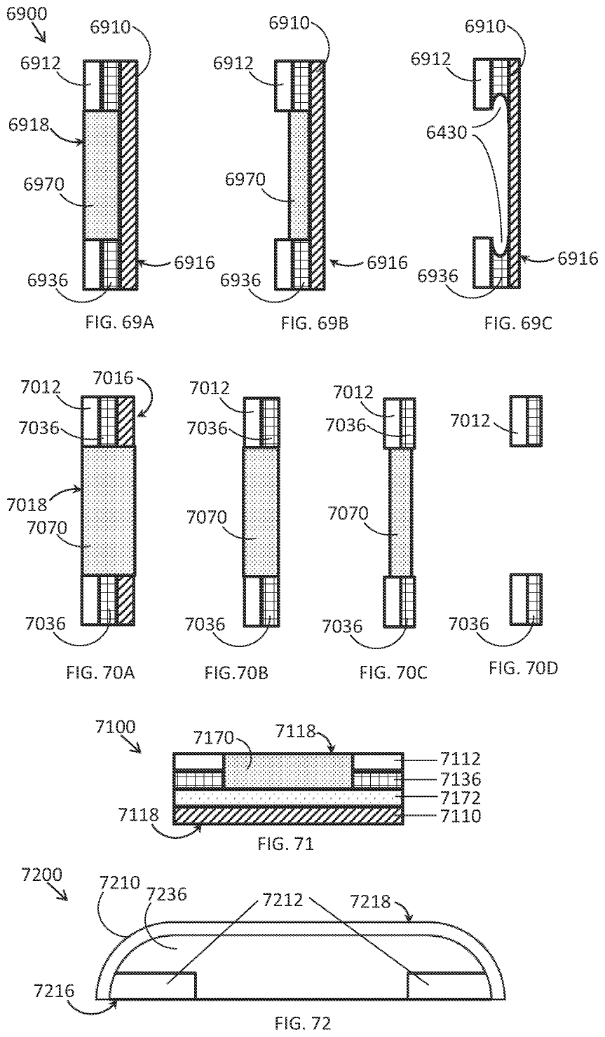

FIGS. 69A-C are simplified schematic cross sections of a device 6900 as residence time of the device progresses, according to some embodiments of the invention.

In some embodiments, FIGS. 69A-C illustrate a portion of a device (e.g. a central portion, where edges are not illustrated).

In FIGS. 69A-C, in some embodiments, like shading indicates portions with the same material characteristics (e.g. lubricious, mucoadhesive, containing therapeutic material).

In some embodiments, device 6900 includes a posterior surface 6916 and an anterior surface 6918, where one or both of surfaces 6916, 6918 have one or more feature (e.g. curvature, thickness) as illustrated and/or described elsewhere in this document.

In some embodiments, device 6900 includes a first mucoadhesive layer 6910, and a second lubricious layer 6912.

Where, in some embodiments, mucoadhesive layer 6910 covers most (e.g. over 55-99%, or 60-90% or lower or higher or intermediate ranges or percentages) or all of posterior surface 6916. A potential benefit of a mucoadhesive layer with a large surface are is increased stability, for example, associated with increased adhesion to the eye surface.

Where, in some embodiments, lubricious layer 6912 covers a portion of anterior surface 6918 e.g. edge region/s.

In some embodiments, device 6900 includes a third layer 6970. Where, in some embodiments, third layer 6970 includes rapidly dissolving and/or degrading and/or abrading material. Where, in some embodiments, the third layer disintegrates more rapidly than other layer/s of the device 6900. For example, as illustrated in transition between FIG. 69A and FIG. 69B where third layer 6970 has reduced in thickness.

In some embodiments, mucoadhesive layer 6910 is also more readily eroded (e.g. associated with proximity to the eye surface which is not illustrated and/or associated with material characteristics of the layer) and/or dissolved and/or degraded (e.g. associated with material characteristics) than one or more other layer of the device. For example, as illustrated in transition between FIG. 69A and FIG. 69B where mucoadhesive layer 6910 has reduced in thickness.

In some embodiments, intermediate layer 6936 includes therapeutic material, where upon being revealed by disappearance of third layer 6970, intermediate layer 6936 begins to dissolve and/or degrade and/or erode, e.g. eluting medication. In some embodiments, intermediate layer is more readily dissolved and/or degraded and/or eroded than one other layer e.g. lubricious layer 6912 and/or mucoadhesive layer 6916 e.g. as illustrated by cavities 6930 having formed in intermediate layer 6936.

FIGS. 70A-D are simplified schematic cross sections of a device as residence time of the device progresses, according to some embodiments of the invention.

In some embodiments, FIGS. 70A-C illustrate a portion of a device (e.g. a central portion, where edges are not illustrated).

In FIGS. 70A-C, in some embodiments, like shading indicates portions with the same material characteristics (e.g. lubricious, mucoadhesive, containing therapeutic material).

In some embodiments, device 7000 has a posterior surface 7016 and an anterior surface 7018, where one or both of surfaces 7016, 7018 have one or more feature (e.g. curvature, thickness) as illustrated and/or described elsewhere in this document.

In some embodiments, device 7000 includes a first mucoadhesive layer 7010, and a second lubricious layer 7012. In some embodiments, device 7000 includes an intermediate therapeutic material 7036 layer.

Where, in some embodiments, one or more than one of (e.g. all of) lubricious layer 7012, mucoadhesive layer 7016, are disposed at edge region/s of device 7000, for example, not being present in a central region of device 7000.

In some embodiments, device 7000 includes a third layer 7070. Where, in some embodiments, third layer 7070 includes rapidly dissolving and/or degrading and/or abrading material. Where, in some embodiments, the third layer disintegrates more rapidly than other layer/s of the device 7000. For example, as illustrated in progression from FIG. 70A, to FIG. 70B, to FIG. 70C where third layer 7070 reduces in thickness and then in transition between FIG. 70C to FIG. 70D where third layer 7070 disappears. In some embodiments, e.g. illustrated in transition between FIG. 70A to FIG. 70B, mucoadhesive layer 7010 disappears first, e.g. exposing intermediate therapeutic material layer 7036. In some embodiments, third layer 7070 disappears leaving one or more remaining layer/s at edges of device e.g. as illustrated in FIG. 70D. In some embodiments, device 7000 is placed over a region of the eye e.g. covering a cornea, where disappearance of third layer 7070 creates a hole in the device e.g. to position device 7000 on sclera surrounding cornea.

FIG. 71 is a simplified schematic cross section of a device 7100, according to some embodiments of the invention.

In some embodiments, FIG. 71 illustrates a portion of a device (e.g. a central portion, where edges are not illustrated).

In FIG. 71, in some embodiments, like shading indicates portions with the same material characteristics (e.g. lubricious, mucoadhesive, containing therapeutic material).

In some embodiments, device 7100 has a posterior surface 7116 and an anterior surface 7118, where one or both of surfaces 7116, 7118 have one or more feature (e.g. curvature, thickness) as illustrated and/or described elsewhere in this document.

In some embodiments, device 7100 includes a first mucoadhesive layer 7110, and a second lubricious layer 7112. In some embodiments, device 7100 includes an intermediate therapeutic material 7136 layer. In some embodiment, mucoadhesive layer 7110 covers most or all of device posterior surface 7116.

Where, in some embodiments, one or more than one of lubricious layer 7112, and intermediate therapeutic material layer 7136, are disposed at edge region/s of device 7100, for example, not being present in a central region of device 7100.

In some embodiments, device 7100 includes a third layer 7170. Where, in some embodiments, third layer 7170 includes rapidly dissolving and/or degrading and/or abrading material. Where, in some embodiments, the third layer disintegrates more rapidly than other layer/s of the device 7100.

In some embodiments, device 7100 includes a degradable layer 7172, which, in some embodiments, degrades more rapidly than one or more of mucoadhesive layer 7110, lubricious layer 7112 and therapeutic layer 7136. Where, in some embodiments, degradable layer 7172 degrades and/or dissolves and/or erodes less rapidly than third layer 7170.

Where, in some embodiments, third layer 7170 is first to disappear, for example, revealing portion/s of therapeutic layer 7136 to initiate and/or increase therapeutic material elution. Where, in some embodiments, degrading layer 7172 then erodes e.g. to further reveal the therapeutic material layer and/or to disintegrate the device.

FIG. 72 is a simplified schematic cross section of a device 7200, according to some embodiments of the invention.

In some embodiments, device 7200 has a posterior surface 7216 and an anterior surface 7218, where one or both of surfaces 7216, 7218 have one or more feature (e.g. curvature, thickness) as illustrated and/or described elsewhere in this document.

In some embodiments, anterior surface 7218 includes a low curvature (or flat) region disposed at a central region of device 7200. In some embodiments, curves towards posterior surface 7216.

In some embodiments, device 7200 includes a first mucoadhesive layer 7210, and a second lubricious layer 7212. In some embodiments, device 7200 includes an intermediate therapeutic material 7236 layer. In some embodiments, second surface 7212 curves around edge/s of device 7200, for example, in some embodiments, forming an edge portion of posterior surface 7216.

In some embodiment, mucoadhesive layer 7210 occupies edge regions of device 7200 and/or posterior surface 7216.

A potential advantage mucoadhesive being disposed locally at edges of device 7200 e.g. as recessions in intermediate therapeutic material 7236 layer is adhesive advantages of mucoadhesive material while maintaining more of a volume of the device for therapeutic material. In some embodiments, intermediate therapeutic material layer 7236 forms at least a part of posterior surface 7216. A potential benefit being ingredient elution rapidly after attachment of device 7200 to an eye surface.

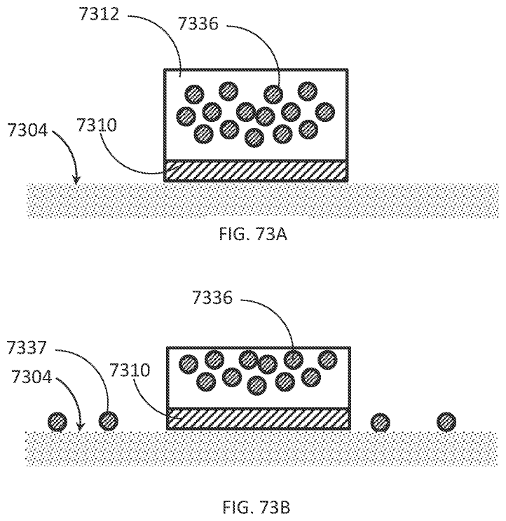

FIGS. 73A-B are simplified schematic cross sectional views of a device 7300 on an eye surface 7304, according to some embodiments of the invention.

For simplicity, device 7300 is illustrated as having a rectangular cross section, however device 7300, in some embodiments, has a shape (shape e.g. including curvature of a posterior and/or anterior surface of the device) having one or more feature of device/s described elsewhere within this document.

In some embodiments, device 7300 includes a layer 7312 which includes therapeutic material particles 7336. Where material particles 7336, in some embodiments, include one or more feature of microparticles and/or nanoparticles e.g. as described elsewhere in this document.

In some embodiments, material particles 7336 are devices having retention portions, for example, as described elsewhere in this document. Where, in some embodiments, device 7300 includes 1-10 particles 7336 e.g. each having a retention portion.

Where, in some embodiments, other material of layer 7312 is rapidly dissolvable and/or degradable and/or erodible. Erosion, in some embodiments, revealing and allowing dispersion of particles 7336, for example, as shown in transition between FIG. 73A to FIG. 73B. In some embodiments, e.g. alternatively to what is illustrated in FIG. 73B, particles are dispersed within the eyelid and remain underneath the eyelid, for example, retention portions meaning that eye tissue retains the particles 7336 underneath the eyelid.

In some embodiments, particles 7336 include therapeutic material. Optionally, in some embodiments particles 7336 include mucoadhesive material. Potentially, once a particle is released from layer 7312 if it contacts eye surface 7304 (e.g. under movement/s of one or more of the eyelid, eyeball, and tear fluid) it adheres to the eye surface. In some embodiments, particles adhere to one or more mucosal surface e.g. including the eyeball and/or eyelid. In some embodiments, the particles then degrade (e.g. over about 10 minutes or about 1 hour, or about 12 hours, or about 1 day, or about 3 days, or lower or higher or intermediate time durations) to release therapeutic material to eye tissue.

Optionally, in some embodiments, device includes one or more additional layer (e.g. than layer 7312). For example, in some embodiments device 7300 includes a mucoadhesive layer 7310.

In some embodiments, therapeutic material layer 7312 including particles is incorporated into one or more of the device embodiments as described within this document. Including, for example, covering and/or access features to therapeutic material layer 7312.

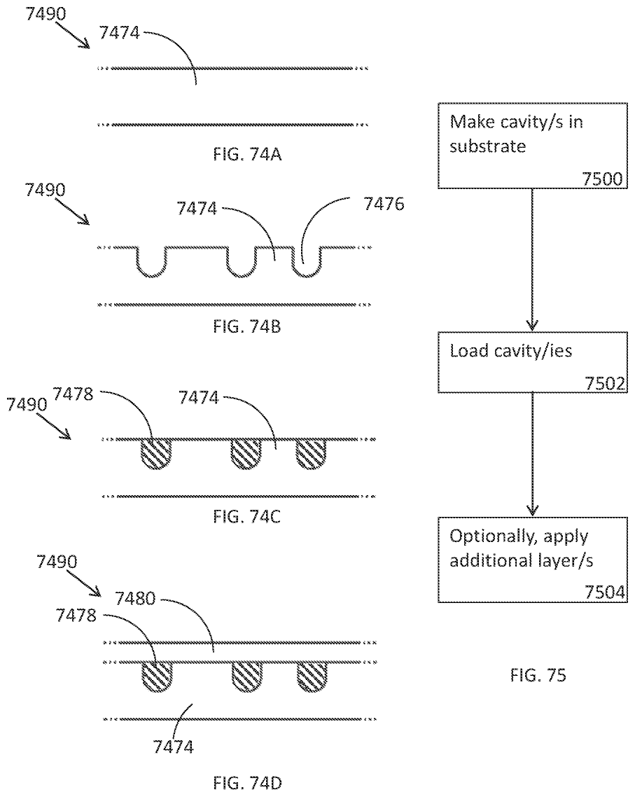

FIGS. 74A-D are simplified schematic cross sectional views of a film, according to some embodiments of the invention.

In some embodiments, FIGS. 74A-D illustrate cross sections views of a portion of a device.

In some embodiments, FIGS. 74A-D show different, portions, for example, sequential portions, of a manufacture process, according to some embodiments of the invention.

FIG. 75 is a method of manufacture, according to some embodiments of the invention.

Referring now to FIG. 74A, in some embodiments, a substrate 7474 is a film e.g. including one or more feature of films as described elsewhere in this document. For example, in some embodiments, the film includes a single layer. For example, in some embodiments, the film includes a plurality of layers e.g. each layer with different composition and/or material characteristics.

At step 300, and, for example, referring to FIG. 74B: In some embodiments, cavity/ies are made in substrate 7474. For example, by and/or using one or more of; Jigs, Press, Punch, Laser engraving, drilling, Molecularly imprinted polymer (MIP), 3D printing.

At step 302, and, for example, referring to FIG. 74C: In some embodiments, cavity/ies are filled e.g. with a different material 7478 to that of substrate 7474.

At step 304, and, for example, referring to FIG. 74D: In some embodiments, one or more additional layer 7480 is applied on top of the substrate layer 7474. In some embodiments, additional layer 7480 has different characteristics (e.g. different material characteristics) than other portion/s 7474, 7478.

In some embodiments, substrate 7474 includes mucoadhesive material, filling material 7478 includes therapeutic material, and additional layer 7480 includes lubricous material.

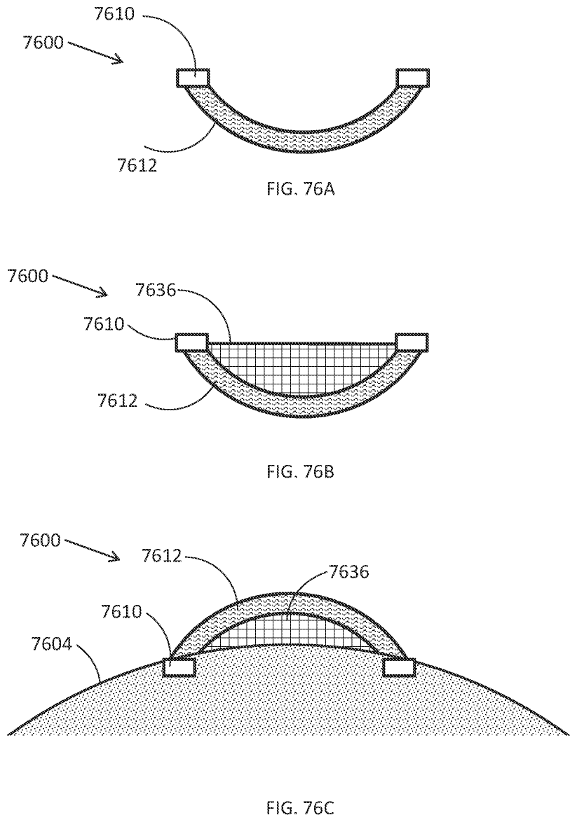

FIGS. 76A-C are simplified schematic cross sectional views of a device 7600, according to some embodiments of the invention.

In some embodiments, device 7600 includes a first layer 7610 and a second layer 7612. In some embodiments, a device posterior surface 7616 is concave. In some embodiments, the concavity is filled with an additional material 7636. For example, during manufacture and/or by a user, for example just prior to use. Where, in some embodiments, additional material 7636 includes therapeutic ingredient/s. Where, in some embodiments, additional material 7636 includes mucoadhesive. Where, in some embodiments, device 7600 is used to extend residence time of a topical application in contact with the eye. For example, where device 7600 seals to an eye surface 7604 holding additional material 7637 in contact with the eye surface e.g. for a time period which is, in some embodiments, longer than residence time of eye drops and/or ointment for example. Where, in some embodiments, the time period is 10 minutes to 12 hours, or 10 minutes to 2 hours, or lower or higher or intermediate time durations or ranges.

In some embodiments, device 7600 includes lubricous material 7612 and/or mucoadhesive material 7610.

### Exemplary Methods of Treatment

In some embodiments, an exemplary method of treating an eye, comprises inserting an ophthalmic device according to any one of the embodiments as disclosed herein, between an eyelid and a surface of said eye; and allowing the release or releasing at least one medication into said eye. In some embodiments, inserting comprises inserting under an inferior eyelid. In some embodiments, inserting comprises inserting under a superior eyelid. In some embodiments, inserting comprises inserting behind a tarsal plate in said eye. In some embodiments, inserting comprises positioning said device on a bulbar conjunctiva and at least partially underneath said eyelid.

### Exemplary Materials

In some embodiments, one or more portion of an ophthalmic device includes one or more of; film forming agent/s, plasticizer/s, binder/s, and excipient/s.

In some embodiments the device contains one or more of anti-infective and anti-inflammatory pharmacotherapies (including: Glucocorticoids, NTHEs, and biological agents or combination of them) and other technologies and means, such as: silver, gold, zinc oxide, titanium dioxide, selenium, copper, polyammonium salts, signaling inhibiting and antimicrobial peptides, cytokines, enzymes.

In some embodiments, the device comprises Thermosensitive and/or PH-sensitive and/or

Ion sensitive compounds.

Without being bound by theory, the molecular weight of the compounds and/or a degree of cross-linking within materials of the device contribute to device consistency (e.g. hardness and/or rigidity) and/or contribute to the device rheological properties (e.g. viscosity). Where, in some embodiments, molecular weight and/or cross-linking and/or consistency and/or rheological properties define the device residence time and/or therapeutic agent release rate.

In some embodiments the device composition comprises at least one charged compound. In some embodiments the device composition comprises at least one charged compound and at least one compound having an opposite charge, constructing a PEC (Poly Electrolyte Complex) formation upon liquid adsorption.

Non-limiting exemplary material/s for:
A non-degradable portion
one or more degradable portion e.g. including
   one or more fast degrading portion
   one or more slower degrading portion
a lubricious portion (e.g. a lubricious layer and/or film) and/or
a mucoadhesive portion (e.g. a mucoadhesive layer and/or film) and/or other portion/s of the ophthalmic device include
one or more of (and/or similar compound/s and/or material/s and/or polymer/s and/or combinations thereof) are herein listed:

Polyethylene-Phthalate polymer film; e.g. purchased as film (MYLAR-A®; DuPont, USA).

Polyvinyl Alcohol 4-88; e.g. purchased as powder (Merck, Germany).

Methocel LV-50; e.g. purchased as powder (DOW, USA).

Poly-Vinyl-Alcohol; e.g. purchased as powder (Merck, Germany).

Poly-Vinyl-Alcohol [PVA]: Poly-Ethylene-Glycol [PEG] purchased as powder (Kollicoat-IR®; BASF, Germany).

graft-copolymer; e.g. e.g.

Hydroxy-Propyl-Cellulose polymer; e.g. purchased as powder (Klucel-LF®; Ashland, USA) Povidone (Kollidone 25; BASF, Germany).

Carbomer, Carbopol, Carbopol 971, Carbopol 974, Carbopol 934, Carbopol 2020.

Cross-linked Poly-Acrylic-Acid; e.g. purchased as powder (Carbopol 974; Lubrizol, USA).

Cross-linked Sodium Polyacrylic-Acid; e.g. purchased as powder (FavorPac®; Evonik, Germany).

Cross-linked Sodium Polyacrylic-Acid; e.g. purchased as powder (Luquasorb®; BASF, USA).

CMC Sodium, Polycarbophil, Tragacanth, Poly acrylic acid polymers, Sodium alginate, (Poly) Hydroxy Ethyl Cellulose, HPMC, Gum Karaya, Gelatin, Guar Gum, Starch, Modified starch, Pectin, Psyllium, Amberlite-Resin, Hydroxy Propyl cellulose.

Chitosan; e.g. purchased as powder (KiOnutrime-CsG; kitozyme, Belgium).

Chitosan; e.g. purchased as powder (Chitoclear; Premix, Iceland or 90/200/A1; Kraeber, Germany or Protasan UP CL 114, 113, 213, 214; Novamatrix, Norway).

Alginate, Gellan gum, Eudragit.

Polyethylene oxide, Carboxymethyl cellulose (CMC), Poly(methyl vinyl ether), Poly(methyl vinyl ether) co maleic anhydride, Hydroxy Propyl methyl cellulose, Methyl Ethyl cellulose, poly hydroxyethyl methacrylate.

Methylcellulose or hypromellose or Povidone (Kollidone 25; BASF, Germany), or polyvinyl pyrrolidone (PVP).

Cross-linked Poly Glutamic-Acid; e.g. purchased as powder (PGA; hayashibira, Japan).

Cross-linked Dextrane gel; e.g. purchased as powder (Sephadex G-100; GE Medical).

Lactose monohydrate; e.g. purchased as powder (Pharmatose 200, Fonterra, New Zealand).

Cellulose acetate, Ethyl cellulose, Methyl cellulose, Modified cellulose.

Silicone, Acrylates, Polyethylenes (including ultra high molecular weight, Polyethylene terephthalate), Polyester.

Polypropylene, Polytetrafluoroethylene (PTFE, ePTFE), Polyether ether ketone (PEEK), Nylon.

Biocompatible metal alloy.

Polymer foam.

Polymethyl methacrylate (PMMA), polyhydroxyethylmethacrylate (pHEMA).

Cellulose acetate butyrate, Siloxane acrylates, t-Butyl Styrene, Fluoro siloxane acrylates, perfluroethers.

Silicone Hydrogel, Lotrafilcon A, Lotrafilcon B, Galyfilcon A, Senofilcon A, Senofilcon C, Sifilcon A, Comfilcon A, Enfilcon A, Balafilcon A, Delefilcon A, Narafilcon B, Narafilcon A, Stenfilcon A, Somofilcon A, Fanfilcon A, Samfilcon A, Elastofilcon.

Tefilcon, Tetrafilcon A, Crofilcon, Helfilcon A/B, Mafilcon, Polymacon, Hioxifilcon B.

Surfilcon A, Lidofilcon A, Lidofilcon B, Netrafilcon A, Hefilcon B, Alphafilcon A, mafilcon A, Omafilcon B, Vasurfilcon A, Hioxifilcon A, Hioxifilcon D, Nelfilcon A, Hilafilcon A, Hilafilcon B, Acofilcon A, Nesofilcon A.

Bufilcon A, Deltafilcon A, Phemfilcon.

Bufilcon A, Perfilcon A, Etafilcon A, Focofilcon A, Ocufilcon B, Ocufilcon C, Ocufilcon D, Ocufilcon E, Ocufilcon F, Phemfilcon A, Methafilcon A, Methafilcon B, Vilfilcon A.

Lotrafilcon A, Balafilcon A, Senofilcon A, Galyfilcon A, Samfilcon A, Comfilcon A, Enfilcon A and similar.

PHEMA (polyhydroxyethilmethacrylate), MA (methacrylic acid), MMA (methyl methacrylate), GMA (glyceryl methacrylate), DAA (diacetone acrylamide, PVOH (polyvinyl alcohol), PVA (Poly-Vinyl-Acetate).

Silicone rubber, Acrylic resins, Polyurethane, Polypropylene, and Polymethylmethacrylate, Polycarbonate urethane, Polyimides.

polyglycolide, polylactide, polyhydroxobutyrate, hyaluronic acid, and hydrogels, poly(2-hydroxyethyl-methacrylate).

polylactic acid (PLA), Polyglycolic acid (PGA), poly(lactic-co-glycolic) acid (PLGA), and poly(caprolactone).

polyglycolide, poly-L-lactide, poly-D-lactide, poly(amino acids), polydioxanone, polycaprolactone, polygluconate, polylactic acid-polyethylene oxide copolymers, polyorthoesters, polyhydroxybutyrate, polyanhydride, polyphosphoester, poly(alpha-hydroxy acid).

polycaprolactone (PCL), polyesteramide (PEA).

Protein, Fatty acids, Amino acids, Carbohydrates, collagen.

Poloxamer, Synperonics, Pluronics, Kolliphor, Pluronic F127 (BASF), Synperonic PE/F 127 (Croda), poloxamer 188 (Pluronic® F-68), poloxamer 407 (Pluronic® F-127), polypropylene glycol, polyoxyethylene, poly(propylene oxide), carboxylated polystyrene, PEGylated polystyrene, dendrimers, Poly(amidoamine) (PAMAM).

Poly-Ethylene-Glycol (Sigma, USA).

Triethyl citrate [TEC] and Acetyl tributyl citrate [ATBC]; e.g. purchased as liquids (Merck, Germany).

Tris(hydroxymethyl)aminomethane.

DEP [Diethyl-Phthalate]; e.g. purchased as liquid (Spectrum, USA).

Glycerol Monostearate [GMS]; e.g. purchased as liquid (Cognis, Germany).

Ethanol, Methanol, Isopropyl Alcohol [IPA], Ethyl Acetate, Acetic-acid, and Acetone (BioLab, Israel) are organic solvents used for the polymers dissolving.

Hypromellose Phthalate [HP-55] and Hypromellose Acetate Succinate [AQOAT AS-LF]; e.g. purchased as powders (Shin-Etsu, Korea) CAP [Cellulose-Acetate-Phthalate] and CA [Cellulose-Acetate]; e.g. purchased as powders (Eastman, USA).

Polysaccharides (β-cyclodextrin, dextrans, inulin, etc.), sugars (glucose, lactose, leucrose, maltose, raffinose, sucrose, trehalose, etc.), Polyol (maltitol, mannitol, sorbitol, xylitol, etc.), oligosaccharides, carbohydrates.

Thermosensitive or PH-sensitive or Ion sensitive pressure sensitive deflection sensitive compounds.

Excipients, including: Antiadherents, Binders and Adhesives (including mucoadhesives), Coatings, Colors, Disintegrants, Flavors, Glidants, Lubricants, Preservatives, Sorbents, Sweeteners, Vehicles.

One or more solvent, for example, one or more of ethanol, methanol, isopropanol, methylene chloride, ethyl acetate, acetone, water, ethanol, polyols (such as glycerol, propylene glycol, polyethylene glycol).

### Exemplary Lubricant Material

In some embodiments, a lubricant portion (e.g. a lubricant layer) includes one or more of; Cellulose Acetate, Ethyl cellulose, PVA (Poly-Vinyl-Alcohol) Poly-Vinyl-Alcohol [PVA]: Poly-Ethylene-Glycol [PEG] graft-copolymer or PVA (Poly-Vinyl-Acetate) or PVA (Poly-Vinyl-Acetate) or Methyl cellulose.

### Exemplary Mucoadhesive Material

In some embodiments, the term “mucoadhesive” encompasses any compound that is capable of adhering to biological tissue. In some embodiments the mucoadhesive is a polymer. In some embodiments the mucoadhesive, in some embodiments, is charged or neutral.

In some embodiments, a mucoadhesive portion (e.g. a mucoadhesive layer) includes one or more ingredient configured to provide mucoadhesive properties.

In some embodiments, a mucoadhesive portion (e.g. a mucoadhesive layer) includes one or more, of: Poly-Vinyl-Alcohol (also called PVA or PVOH), hydroxypropylcellulose (Klucel), Polyvinyl alcohol-polyethylene glycol graft-copolymer (Kollicoat-IR), methylcellulose, hypromellose methylcellulose, hydroxypropyl methylcellulose (Methocel), Povidone (polyvinylpyrrolidone).

### Exemplary Therapeutic Agents

In some embodiments, a therapeutic material portion or layer of a device according to embodiments described in this document, contains one or more therapeutic agents, for example, one or more drug and/or natural compound or synthetic compound.

In some embodiments, one or more therapeutics agents is in one or more of solid form, powder, ointment, liquid, bids (e.g. polymer bids), dispersion, solution, pellets, microparticles, microspheres, and microcapsules.

In some embodiments, therapeutic material contains one or more therapeutic agents as microparticles in the 1-1000 μm size range.

In some embodiments, therapeutic material contains active pharmaceutical ingredients (API) and/or excipients and/or combinations thereof.

In some embodiments, an ophthalmic device (e.g. according to one or more embodiment as described in this document) contains a dose of one or more therapeutic agent within a range from about 1 μg to about 10,000 μg, or above 10,000 μg, or between 0 and 1 μg, or between 1 μg and 5 μg, or between 5 μg and 10 μg, or between 10 μg and 50 μg, or between 50 μg and 100 μg, or between 100 μg and 500 μg, or between 500 μg and 1000 μg, or between 1000 μg and 5000 μg, or between 5000 μg and 10000 μg, or between 10000 μg and 30000 μg.

In some other embodiments, an ophthalmic device (e.g. according to one or more embodiment as described in this document) contains a mass of therapeutic agent of between about 1 microgram to about 20 milligrams.

In some embodiments, the device is about 0.1% to about 99.5%, or about 0.5% to about 90%, drug release material e.g. in combination with pharmaceutically acceptable carrier material/s.

In some embodiments, the device has a concentration of therapeutic agent less than 0.01%, or between 0.01% and 0.1%, or between 0.1% and 1%, or between 1% and 5%, or between 5% and 10%, or between 10% and 30%, or between 30% an 60%, or between 60% and 90%, or between 90% and 100%.

In some embodiments, the ophthalmic device (e.g. a therapeutic material layer and/or region) includes one or more of the following therapeutic agents (also herein termed “therapeutic ingredients”):

Aflibercept, Atropine, Bevacizumab, Brimonidine/timolol, Ciclosporin, Ciprofloxacin, Cyclopentolate, Diisopropyl fluorophosphate, Diquafosol, Dorzolamide/timolol, chothiophate, Latanoprost/timolol, Latanoprostene bunod, Lifitegrast,

Loteprednol, fluocinolone, fluorometholone, difluprednate, prednisolone, triamcinolone, rimexolone

Mydriatics and cycloplegics, Nandrolone sulfate, Naphazoline/pheniramine, Nedocromil, Netarsudil, Ofloxacin, Pegaptanib, Pilocarpine, Pirenoxine, Ranibizumab, Ripasudil, Tauroursodeoxycholic acid, Tavilermide, Tropicamide/hydroxyamfetamine, Verteporfin

Acthar, Bevacizumab, Cefazolin, Ceftazidime, Cocaine, EDTA, Gatifloxacin, Gentamicin, Idoxuridine, Iluvien®, Indocyanine Green, Infliximab, Interferon, Intravitreal Injections, Iohexol, Iopamidol, Iopamidol-M, Lissamine Green, Membrane Blue, Mitomycin, Moxifloxacin, Rituximab, Serum eye drops, Single-Use Povidone, Subconjunctival Injections, Tetracaine PF, Tobramycin, TPA, Vancomycin

Cyclopentolate, Phenylephrine, Proparacaine, Steroids, Tropicamide Amikacin, Amphotericin-B, Dexamethasone, Mitomycin Small molecules, Biological drugs, autoimmune drugs, anti-cancer drugs and/or agents, anti-inflammatory agents, anti-infection agents, antibiotics, cannaboids

### Synthetic Compounds

### Natural Compounds

Drug, in some embodiments is hydrophilic or hydrophobic

Drug formulation, in some embodiments is hydrophilic or hydrophobic

Excipients, including: Antiadherents, Binders, Coatings, Colors, Disintegrants, Flavors, Glidants, Lubricants, Preservatives, Sorbents, Sweeteners, Vehicles

In some embodiments, a therapeutic material includes enhancer materials for example: for penetration enhancement and/or for membrane pore openers.

In some embodiments, a therapeutic material includes materials for improved solubility in human fluids (including serum and/or blood)

In some embodiments, a therapeutic material includes prodrug form of another drug.

In some embodiments, a therapeutic material includes a biosimilar form of another drug.

The therapeutics agent, in some embodiments is at any dosage form including modified release dosage form such as: immediate-release dosage, delayed-release dosage, extended-release dosage, sustained-release dosage, stimuli inducing release and/or targeted-release dosage.

Techniques for modified release dosage, in some embodiments is using any of the following release approaches: Diffusion, Dissolution, Osmotic, Ion-exchange resin, Floating, Bio-adhesive, Matrix, etc.

Processes for modified release form include: microencapsulation, molecularly imprinted polymer (MIP), 3D drug printing

The therapeutics agent, in some embodiments is at any formulation contains any excipient and other compound available.

Exemplary Nanoparticle and/or Microparticle Therapeutic Agent/s

In some embodiments, a therapeutic material portion or layer of a device (e.g. according to one or more embodiment described in this document) contains one or more therapeutic agent in the form of nanoparticles and/or microparticles. In some embodiments, nanoparticle and/or microparticle therapeutic agent/s are covered and/or contain mucoadhesive compounds and/or polymer/s. Where, in some embodiments, covering of the particles/s increases residence time and/or duration of drug elution of the particle/s.

For example, Atropine microparticles.

For example, atropine sulfate in albumin-chitosan microparticles e.g. including one or more feature as described in “Formulation and characterization of atropine sulfate in albumin-chitosan microparticles for in vivo ocular drug delivery” by Richard T Addo et al, J Pharm Sci. 2015 May; 104 (5): 1677-90, which is herein incorporated by reference in its entirety.

Where, in some embodiments, microparticles have a mean size of about 2 microns, atropine concentration of 10% and a release time of about 50 hours. Such particles, in some embodiments are over coated with additional polymer/s with one or more different properties to achieve, in some embodiments, an accumulated extended release pattern over 7 days or longer.

For example, microparticles including one or more feature as described in “Development of Water-Compatible Molecularly Imprinted Polymers Based on Functionalized β-Cyclodextrin for Controlled Release of Atropine” by Yahui He et al, Polymers 2020, 12 (1), 130, which is herein incorporated by reference in its entirety.

For example, Timolol nanoparticles.

For example, particles including one or more feature as described in “Chitosan nanoparticles for prolonged delivery of timolol maleate” by Sunil A Agnihotri 1, Tejraj M Aminabhavi, Drug Dev Ind Pharm. 2007 November; 33 (11): 1254-62, which is herein incorporated by reference in its entirety.

In some embodiments, the particles have a mean particle size ranged between 118 and 203 nm, while, in some embodiments, zeta potential ranges between +17 and +22 mV. In some embodiments, entrapment efficiency of the nanoparticles ranges between 47.6 and 63.0%. In some embodiments, e.g. based on results in-vitro release studies performed in phosphate buffer saline of pH 7.4, release of timolol maleate is slow, e.g. by up to 24 hours. The particles, in some embodiments are over coated with additional polymer/s e.g. with different properties to arrive at an accumulated extended release pattern over 7 days or longer.

For example, timolol microparticles including one or more feature as described in “Sustained delivery of timolol malcate from poly(lactic-co-glycolic acid)/poly(lactic acid) microspheres for over 3 months” by James P Bertram 1, Sandeep S Saluja, Jodi McKain, Erin B Lavik, J Microencapsul. 2009 February; 26 (1): 26, which is herein incorporated by reference in its entirety.

In some embodiments, microspheres are fabricated using a 50:50 blend of PLGA 502H and PLA. In some embodiments, the microspheres deliver timolol maleate over 50-200 days, or 50-150 days, or 90-120 days, or about 107 days.

### General

It is expected that during the life of a patent maturing from this application many relevant ophthalmic treatments and/or ophthalmic devices and/or therapeutic ingredients will be developed and the scope of the terms ophthalmic treatment and/or ophthalmic device and/or therapeutic ingredients is intended to include all such new technologies a priori.

As used herein the term “about” refers to ±20%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. An ophthalmic device comprising:
a body comprising:
a posterior surface which is adjacent to an eye surface when said ophthalmic device is worn;
an anterior surface which is adjacent to an eyelid when said ophthalmic device is worn;
a retention portion defined by heights between said posterior surface and said anterior surface;
wherein a first height of said heights measured from a first point on said posterior surface to a corresponding point on said anterior surface differs by at least 1 mm, from a second height of said heights measured from a second point on said posterior surface to a corresponding point on said anterior surface, said second point being distanced at most 1 mm from said first point.

2. The ophthalmic device according to claim 1, wherein said body is not hollow.

3. The ophthalmic device according to claim 1, wherein said retention portion is made of a continuous material.

4. The ophthalmic device according to claim 1, wherein said body has an oval or round form.

5. The ophthalmic device according to claim 1, wherein said body is symmetrical in its longitudinal axis and in its transversal axis.

6. The ophthalmic device according to claim 1, wherein said retention portion has a height of at least 1 mm for an area of at least 1 mm2.

7-13. (canceled)

14. The ophthalmic device according to claim 1, wherein said retention portion has a slope of at least 45° for at least a length of 1 mm along said anterior surface.

15. The ophthalmic device according to claim 1, wherein said body comprises a first portion and a second portion, where said first portion degrades more rapidly than said second portion when said body is in residence on said eye surface.

16. The ophthalmic device according to claim 15, wherein said second portion forms part or all of said retention portion.

17-19. (canceled)

20. The ophthalmic device according to claim 1, wherein, when in residence on said eye surface, said anterior surface is adjacent to an inner surface of said eyelid, and said retention portion is held in position by a tarsal plate.

21. (canceled)

22. The ophthalmic device according to claim 1, wherein an extent of said body is 2-8 mm.

23. The ophthalmic device according to claim 1, wherein said body is elongate.

24. The ophthalmic device according to claim 1, wherein said body is rotationally symmetrical around an axis connecting said anterior surface and said posterior surface.

25-26. (canceled)

27. The ophthalmic device according to claim 1, wherein said posterior surface is concave.

28-29. (canceled)

30. The ophthalmic device according to claim 1, wherein said device is configured to move with respect to said eye surface, during wear.

31. The ophthalmic device according to claim 30, wherein said posterior surface has sufficiently low suction forces onto said eye surface to enable said movement.

32-40. (canceled)

41. The ophthalmic device according to claim 1, wherein said body comprises more than one layer with different material characteristics.

42-45. (canceled)

46. The ophthalmic device according to claim 1, wherein said device comprise a section comprising a length of at least 1 mm where a height between said anterior surface and said posterior surface is constant.

47. (canceled)

48. The ophthalmic device according to claim 1, wherein said retention portion prevents said device from sliding under an upper eyelid.

49. (canceled)

50. An ophthalmic device, comprising:
A body comprising:
a. a posterior surface which is adjacent to an eye surface when said ophthalmic device is worn;
b. an anterior surface which is adjacent to an eyelid when said ophthalmic device is worn;
c. a retention portion defined by heights between said posterior surface and said anterior surface;
wherein at least some of said heights between said posterior surface and said anterior surface differ from each other by at least 1 mm when measured from two points on said posterior surface distanced from each other by at most 1 mm.
